(19) United States
(12) Patent Application Publication
(10) Pub. No.: US 2010/0130359 A1
(43) Pub. Date: May 27, 2010

(54) Fungicidal Pyridazines, Processes for Their Preparation and Their Use for Controlling Harmful Fungi, and Compositions Comprising Them

(75) Inventors: Jochen Dietz, Mannheim (DE); Marianna Vrettou, Mannheim (DE); Bernd Mueller, Frankenthal (DE); Jens Renner, Bad Duerkheim (DE); Sarah Ulmscheneider, Bad Duerkheim (DE); Jan Klaas Lohmann, Ludwigshafen (DE)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/597,972

(22) PCT Filed: Apr. 25, 2008
(86) PCT No.: PCT/EP08/55058

§ 371 Date: Oct. 28, 2009

(30) Foreign Application Priority Data
May 2, 2007 (EP) 07107339.9

(51) Int. Cl. A01C 1/06; C07D 401/04; A61K 31/50; A01N 43/58; C07D 237/02

(52) U.S. Cl. 504/100; 544/238; 514/252.03; 514/247; 544/224

(57) ABSTRACT

Pyridazines of the formula I

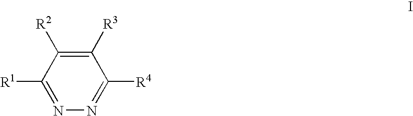

in which the substituents are defined according to the description, processes and intermediates for preparing these compounds, compositions comprising them and also their use for controlling phytopathogenic harmful fungi, and also the use of the pyridazines for preparing a medicament for treating cancer.

The present invention relates to pyridazines of the formula I

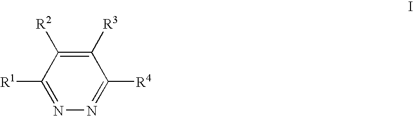

in which the substituents have the following meaning:
R¹, R⁴ independently of one another are halogen, cyano, C₁-C₈-alkyl or C₁-C₈-alkoxy;
R² is C₁-C₁₂-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₆-cycloalkyl, C₃-C₈-cycloalkenyl, C₃-C₁₂-cycloalkenyl, C₃-C₆-halocycloalkyl, C₃-C₁₂-halocycloalkenyl, aryl or aromatic heterocycle which, in addition to carbon atoms, contains one to four nitrogen atoms or one to three nitrogen atoms and one sulfur or oxygen atom as ring members, where the aromatic groups are five-, six-, seven-, eight-, nine- or ten-membered ring systems; and R² may contain one, two, three or four identical or different groups R^{a} independently of one another selected from the group consisting of:
   R^{a} is cyano, nitro, hydroxyl, carboxyl, C₁-C₆-alkyl, C₂-C₆-alkynyl, C₃-C₆-cyclo-alkyl, C₃-C₈-cycloalkenyl, C₁-C₆-alkoxy, C₂-C₆-alkenyloxy, C₃-C₆-alkynyloxy, C₃-C₆-cycloalkoxy, C₃-C₆-cycloalkenyloxy, C(O)R^{π}, C(O)OR^{π}, C(S)OR^{π}, C(O)SR^{π}, C(S)SR^{π}, OC(O)OR^{π}, C₁-C₆-alkylthio, amino, C₁-C₆-alkylamino, di-C₁-C₆-alkylamino, C₁-C₆-alkylene, oxy-C₁-C₄-alkylene, oxy-C₁-C₃-alkyleneoxy, where divalent groups may be attached to the same atom or to adjacent atoms, phenyl, naphthyl, a five-, six-, seven-, eight-, nine- or ten-membered saturated, partially unsaturated or aromatic heterocycle which, in addition to carbon atoms, contains one to four nitrogen atoms or one to three nitrogen atoms and one sulfur or oxygen atom as ring members;
      R^{π} is C₁-C₈-alkyl, C₃-C₈-alkenyl, C₃-C₈-alkynyl, C₃-C₆-cycloalkyl or C₃-C₆-cycloalkenyl;
         where the aliphatic, alicyclic or aromatic groups in groups R^{a} and R^{π} mentioned above for their part may be partially or fully halogenated and/or may carry one, two or three groups R^{b}:
      R^{b} is halogen, cyano, nitro, hydroxyl, mercapto, amino, carboxyl, alkyl, haloalkyl, alkenyl, alkoxy, haloalkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, where the alkyl groups in these groups contain 1 to 6 carbon atoms and the alkenyl or alkynyl groups mentioned in these groups contain 2 to 8 carbon atoms; cycloalkyl, cycloalkoxy, heterocyclyl, heterocyclyloxy, where the cyclic systems contain 3 to 10 ring members; aryl, aryloxy, arylthio, aryl-C₁-C₆-alkoxy, aryl-C₁-C₆-alkyl, hetaryl, hetaryloxy, hetarylthio, where the aryl radicals preferably contain 6 to 10 ring members and the hetaryl radicals 5 or 6 ring members, where the cyclic systems may be partially or fully halogenated and/or substituted by alkyl or haloalkyl groups;
R³ is phenyl or a 5- or 6-membered heteroaromatic group which, in addition to carbon atoms, contains 1, 2, 3 or 4 heteroatoms selected from the group consisting of O, S and N as ring members, where phenyl or the heteroaromatic group carries one substituents L¹ and optionally substituents Lₘ;
   L¹ is a group of the formulae —Y¹—Y²-T, C(Rⁱ)═C(Rⁱⁱ)—Y¹—Y²-T or C≡C—Y¹—Y²-T, in which
      Y¹ is CR^{h}Rⁱ, C(O)O, C(O)NR^{h}, O, NR^{h} or S(O)ᵣ;
      Y² is C₁-C₈-alkylene, C₂-C₈-alkenylene or C₂-C₈-alkynylene, where r may be interrupted by one, two, three or four heteroatoms from the group consisting of NR^{h}, O and S(O)ᵣ and/or may contain one, two, three or four identical or different groups R^{a};
         r is 0, 1 or 2;
      T is OR^{h}, NR^{h}Rⁱ, C(O)OR^{h}, C(O)NR^{h}Rⁱ, C(NOR^{h})Rⁱ or T¹-C(=T²)-T³, where
      T¹ is O or NR^{h};
      T² is O, S or NR^{h};
      T³ is R^{h}, OR^{h}, SR^{h} or NR^{h}Rⁱ;
         independently, each R^{h} is hydrogen or has one of the meanings mentioned for R^{π}; and
   Rⁱ, Rⁱⁱ independently of one another are one of the groups mentioned for R^{a}; L independently of one another are halogen, hydroxyl, mercapto (SH), cyanato (OCN), cyano, nitro, C₁-C₈-alkyl, C₂-C₁₀-alkenyl, C₂-C₁₀-haloalkenyl, C₂-C₁₀-alkynyl, C₃-C₆-cycloalkyl, C₃-C₆-halocycloalkyl, C₃-C₆-cycloalkenyl, C₁-C₈-alkoxy, C₁-C₈-haloalkoxy, C₂-C₁₀-alkenyloxy, C₂-C₁₀-alkynyloxy, C₃-C₆-cycloalkyloxy, C₃-C₆-cycloalkenyloxy, amino, C₁-C₄-alkylamino, di-(C₁-C₄)-alkylamino, C(O)—R^{Φ}, C(S)—R^{Φ}, S(O)ₙ—R^{Φ}; C₁-C₈-alkoxyimino-(C₁-C₈)-alkyl, C₂-C₁₀-alkenyloxyimino-(C₁-C₈)-alkyl, C₂-C₁₀-alkynyloxyimino-(C₁-C₈)-alkyl, C₂-C₁₀-alkynylcarbonyl, C₃-C₆-cycloalkylcarbonyl, or a five-, six-, seven-, eight-, nine- or ten-membered saturated, partially unsaturated or aromatic heterocycle which contains one, two, three or four heteroatoms from the group consisting of O, N and S;
      R^{Φ} is hydrogen, C₁-C₄-alkyl, C₁-C₂-haloalkyl, C₁-C₄-alkoxy, C₂-C₄-alkenyloxy, C₂-C₄-alkynyloxy; where the groups R^{Φ} may be substituted by one, two or three identical or different groups R^{b}, as defined above;
      n is zero, 1 or 2;
   m is 1, 2, 3 or 4;

and agriculturally acceptable salts thereof.

Moreover, the invention relates to processes and intermediates for preparing these compounds, to compositions comprising them and also to their use for controlling phytopathogenic harmful fungi. Moreover, the invention provides the use of the pyridazines for preparing a medicament for treating cancer.

WO 2005/121104 and WO 2006/045192 disclose 3,6-dimethyl-4,5-diphenylpyridazines and 3-chloro-6-methyl-4,5-diphenylpyridazines, respectively, having fungicidal action. Individual 3,6-dichloro-4,5-diphenylpyridazines and 3,6-dichloro-4-methyl-5-phenylpyridazines are disclosed in WO 2005/063762, J. Org. Chem. Vol. 29, pp. 2128-35 (1964), J. Chem. Soc., p. 1316 (1970).

In many cases, in particular at low application rates, the fungicidal action of the known compounds is unsatisfactory. Accordingly, it is an object of the present invention to provide compounds having improved action and/or a broader activity spectrum.

Accordingly, we have found the compounds defined at the outset. Furthermore, we have found processes and intermediates for their preparation, compositions comprising them and also methods for controlling harmful fungi using the compounds I.

The compounds of the formula I according to the invention can be obtained by different routes.

Pyridazines of the formula I in which both groups R¹ and R⁴ are halogen, in particular chlorine, are advantageously accessible by halogenation of the compounds of the formula II with a halogenating agent [HAL] by the route below. They correspond to the formula I.1, in which Hal is halogen, preferably bromine or chlorine, in particular chlorine.

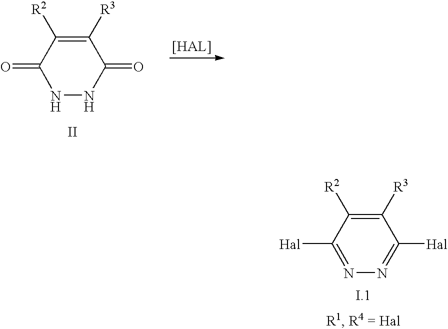

This reaction is usually carried out at temperatures of from 0 to 150° C. or preferably from 80 to 125° C., in the absence of a solvent or in an inert organic solvent [cf. Bioorg Med Chem Lett, 2003, 13, 1581; J Chem Soc, 1970, 1316]. Preferred halogenating agents are chlorinating or brominating agents, such as phosphorus oxybromide, phosphorus oxychloride, thionyl chloride, thionyl bromide or sulfuryl chloride, in particular phosphorus oxychloride. The reaction can be carried out in the absence of a solvent or in the presence of a solvent.

The halogenating agent is generally employed in equimolar amounts. It may also be used in excess or as solvent.

Suitable solvents are aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide. It is also possible to use mixtures of the solvents mentioned.

The 3,6-dihydroxypyridazines of the formula II are preferably obtained by reacting furandiones of the formula III with hydrazine or hydrazine hydrate.

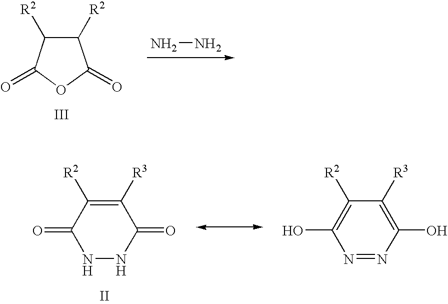

This reaction is usually carried out at temperatures of from −30° C. to 150° C., preferably from 50° C. to 120° C., in an inert organic solvent or in suitable inorganic or organic acids and also water [cf. Eur J Org Chem, 2004, 2797-2804; J Chem Soc, 1970, 1316; WO 2006/032518; Heteroatom Chem, 16(4), 298-307, 2005; Hely Chim Acta, 85 (7), 2195-2213, 2002].

Suitable solvents are ethers, such as diethyl ether, diisopropyl ether, tert-butyl methyl ether, dioxane, anisole and tetrahydrofuran, alcohols, such as methanol, ethanol, n-propanol, isopropanol, n-butanol and tert-butanol. It is also possible to use mixtures of the solvents mentioned.

Suitable for use as acids are inorganic acids, such as hydrofluoric acid, hydrochloric acid, hydrobromic acid, sulfuric acid and perchloric acid, and also organic acids, such as formic acid, acetic acid, propionic acid, oxalic acid, toluenesulfonic acid, benzenesulfonic acid, camphorsulfonic acid, citric acid and trifluoroacetic acid.

The starting materials are generally reacted with one another in equimolar amounts. In terms of yield, it may be advantageous to employ the hydrazine or hydrazine hydrate in an excess of up to 5 molar equivalents, based on III.

The furandiones of the formula III are advantageously obtained by oxidative cyclization of esters of the formula IV.

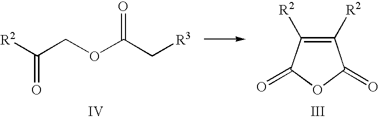

This reaction is usually carried out at temperatures of from ±30° C. to +100° C., preferably from +10° C. to +50° C., in an inert organic solvent in the presence of a base [cf. Synlett, 2002, (6), 947-951; Bioorg Med Chem Lett, 2003, 13, 1195]. A suitable oxidizing agent is, for example, oxygen.

Suitable solvents are aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide, particularly preferably nitriles, such as acetonitrile and propionitrile. It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, sodium amide and potassium amide, alkali metal and alkaline earth metal carbonates, such as lithium carbonate, potassium carbonate and calcium carbonate, and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide and dimethoxymagnesium, moreover organic bases, for example tertiary amines, such as trimethylamine, triethylamine, diisopropylethylamine and N-methylpiperidine, pyridine, substituted pyridines, such as collidine, lutidine and 4-dimethylaminopyridine, and also bicyclic amines, such as 1,8-diazabicyclo[5.4.0]undec-7-ene (DBU). Particular preference is given to DBU. The bases are generally employed in catalytic amounts; however, they can also be employed in equimolar amounts, in excess or, if appropriate, as solvent.

The starting materials are generally reacted with one another in equimolar amounts. In terms of yield, it may be advantageous to employ about 3 molar equivalents of the base, based on IV.

The esters of the formula IV can be obtained by condensation of carboxylic acids of the formula V or sodium or potassium salts thereof with halides of the formula VI in which X is a halogen, such as iodine, chlorine or bromine, preferably chlorine or bromine.

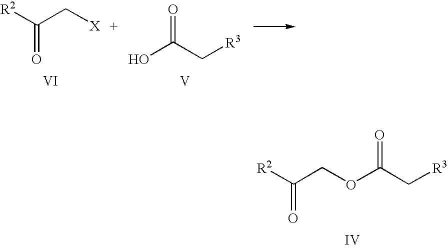

This reaction is usually carried out at temperatures of from −50° C. to 100° C., preferably from 25° C. to 100° C., in an inert organic solvent in the presence or absence of a base [cf. Bioorg Med Chem Lett, 2003, 13, 1195; Synth. Commun., 25(12), 1681-1686, 2005, Org Prep and Proc Int, 20(5), 527-532, 1988; Synth Commun, 16 (14), 1777-1780, 1986].

Suitable solvents are water, aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide, particularly preferably acetonitrile. It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, sodium amide and potassium amide, alkali metal and alkaline earth metal carbonates, such as lithium carbonate, potassium carbonate and calcium carbonate, and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide and dimethoxymagnesium, moreover organic bases, for example tertiary amines, such as trimethylamine, triethylamine, diisopropylethylamine, trisdioxaheptylamine (TDA) and N-methylpiperidine, pyridine, substituted pyridines, such as collidine, lutidine and 4-dimethylaminopyridine, and also bicyclic amines. Particular preference is given to bases, such as potassium carbonate and TDA.

The bases are generally employed in catalytic amounts; however, they can also be employed in equimolar amounts, in excess or, if appropriate, as solvent.

Halides of the formula VI in which X is a halogen, such as iodine, bromine or chlorine, in particular chlorine or bromine, can be obtained by halogenation of ketones of the formula VII.

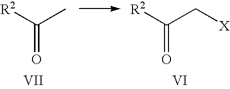

This reaction is usually carried out at temperatures of from −78° C. to 100° C., preferably from 25° C. to 80° C., in an inert organic solvent or in an acid [cf. JACS, 1997, 119, 2453-2463; Synthesis (2), 143-146, 1980; JACS, 1208 (8), 2558-2570, 2006]. Preferred halogenating agents are elemental halogen, such as chlorine or bromine, in particular bromine, or other customary halogenating agents, such as N-bromosuccinimide, thionyl chloride, sulfuryl chloride, Cu(2) bromide or benzyltrimethylammonium tribromide.

Suitable solvents are ethers, such as diethyl ether, diisopropyl ether, tert-butyl methyl ether, dioxane, anisole and tetrahydrofuran (THF), particularly preferably THF, or halogenated hydrocarbons, such as methylene chloride, chloroform and carbon tetrachloride, but also other solvents, such as, for example, ethyl acetate.

Suitable acids are organic acids, such as formic acid, acetic acid, propionic acid, oxalic acid, toluenesulfonic acid, benzenesulfonic acid, camphorsulfonic acid, citric acid and trifluoroacetic acid. It is also possible to use mixtures of the solvents mentioned.

Compounds of the formula I in which both groups R¹ and R⁴ are halogen, in particular chlorine, and R² is an aliphatic group, can alternatively be obtained from furandiones of the formula III, which can be obtained by reacting α-ketocarboxylic acids or activated derivatives thereof, such as, for example, esters of the formula VIII in which R is C₁-C₄-alkyl, such as methyl or ethyl, with carboxylic anhydrides of the formula IX.

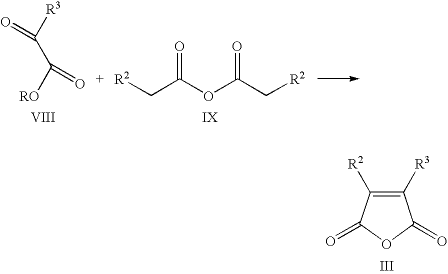

This reaction is usually carried out at temperatures of from 0° C. to 150° C., preferably from 0° C. to 50° C., in an inert organic solvent in the presence of a base and also a catalyst [cf. Tetrahedron Lett, 47 (2006), 2107-2109].

Suitable solvents are aliphatic hydrocarbons, such as pentane, hexane, cyclohexane and petroleum ether, aromatic hydrocarbons, such as toluene, o-, m- and p-xylene, halogenated hydrocarbons, such as methylene chloride, 1,2-dichloroethane, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide, particularly preferably dichloromethane. It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, sodium amide and potassium amide, alkali metal and alkaline earth metal carbonates, such as lithium carbonate, potassium carbonate and calcium carbonate, and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide and dimethoxymagnesium, moreover organic bases, for example tertiary amines, such as trimethylamine, triethylamine, tributylamine, diisopropylethylamine and N-methylpiperidine, pyridine, substituted pyridines, such as collidine, lutidine and 4-dimethylaminopyridine, and also bicyclic amines. Particular preference is given to tributylamine. The bases are generally employed in catalytic amounts; however, they can also be employed in equimolar amounts, in excess or, if appropriate, as solvent.

Suitable acids and acidic catalysts are Lewis acids, such as boron trifluoride, aluminum trichloride, iron(III) chloride, tin(IV) chloride, titanium(IV) chloride and zinc(II) chloride, preferably titanium(IV) chloride.

α-Ketocarboxylic esters of the formula VIII can preferably be obtained under Grignard conditions from the appropriate (hetero)aryl halides, in particular (hetero)aryl bromides, of the formula R³—X in which X is halogen, such as chlorine or bromine, in particular bromine, and the appropriate dialkyl oxalates of the formula XI, in particular diethyl oxalate.

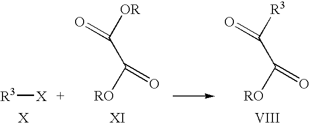

This reaction is in the first step usually carried out at temperatures of from −100° C. to 0° C., preferably from −80° C. to −20° C., in the absence of a solvent or in an inert organic solvent in the presence of a Grignard reagent or in the presence of magnesium [cf. lit. Synlett, (6), 885-887, 2003; J Med Chem, 49 (4), 1271-1281, 2006]. Suitable Grignard salts are, preferably, C₁-C₄-alkylmagnesium chlorides, in particular isopropylmagnesium chloride.

Suitable solvents are the dialkyl oxalate of the formula XI, aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran (THF), nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide, particularly preferably ethers, such as diethyl ether or THF. It is also possible to use mixtures of the solvents mentioned.

The reaction of the Grignard reagent, formed in the first step, with the oxalic esters is usually carried out at temperatures of from −78° C. to +100° C., preferably from +10° C. to +50° C., in an inert organic solvent [cf. lit. J Med Chem, 49, (4), 1271-1281, 2006; JOC, 68 (10), 3990-3998, 2003].

Suitable solvents are aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide, particularly preferably ethers, such as diethyl ether or THF and aliphatic hydrocarbons, such as, for example, hexane. It is also possible to use mixtures of the solvents mentioned.

Furthermore, the α-ketocarboxylic esters of the formula VIII can be prepared directly from oxalic esters of the formula XI and the appropriate aryl halides of the formula X in the presence of a base [cf. JACS, 125 (30), 9032-9034, 2003; Synthesis, 9, 1241-1242, 1998]

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, sodium amide and potassium amide, alkali metal and alkaline earth metal carbonates, such as lithium carbonate, potassium carbonate and calcium carbonate, and also alkali metal bicarbonates, such as sodium bicarbonate, organometallic compounds, in particular alkali metal alkyls, such as methyllithium, butyllithium and phenyllithium, particularly preferably butyllithium, alkylmagnesium halides, such as methylmagnesium chloride, and also alkali metal and alkaline earth metal alkoxides, such as sodium methoxide, sodium ethoxide, potassium ethoxide, potassium tert-butoxide and dimethoxymagnesium, moreover organic bases, for example tertiary amines, such as trimethylamine, triethylamine, tributylamine, diisopropylethylamine and N-methylpiperidine, pyridine, substituted pyridines, such as collidine, lutidine and 4-dimethylaminopyridine, and also bicyclic amines. The bases are generally employed in catalytic amounts; however, they can also be employed in equimolar amounts, in excess or, if appropriate, as solvent.

Compounds of the formula I in which one of the groups R¹ and R⁴ is alkoxy and the respective other group is halogen, in particular chlorine, correspond to the formulae I.2 and I.3, respectively. They can be obtained by reacting the compounds of the formula I.1 with the appropriate alkoxide [Y⁻—⁻M⁺].

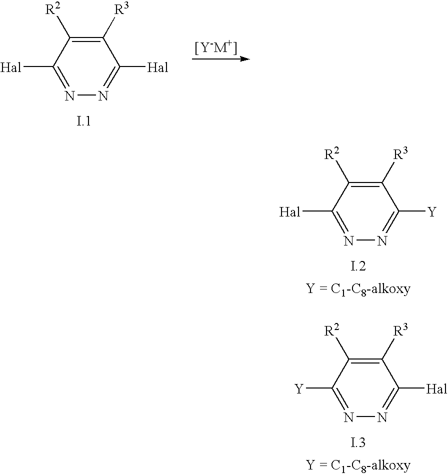

This reaction is usually carried out at temperatures of from 0° C. to 120° C., preferably from 25° C. to 100° C., in an inert organic solvent [cf. Tetrahedron, 60(36), 7983-7994, 2004; Synthesis, (7), 1163-1168, 1999].

Suitable solvents are aliphatic hydrocarbons, such as pentane, hexane, cyclohexane and petroleum ether, aromatic hydrocarbons, such as toluene, o-, m- and p-xylene, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide, particularly preferably alcohols, such as methanol, ethanol, n-propanol, isopropanol, n-butanol and tert-butanol, in particular methanol. It is also possible to use mixtures of the solvents mentioned.

The starting materials are generally reacted with one another in equimolar amounts. In terms of yield, it may be advantageous to employ an excess of Y+M⁻, based on I.1.

Compounds of the formula I in which both groups R¹ and R⁴ are alkoxy correspond to the formula I.4. They can be obtained by reacting the compounds of the formula I.1 with the appropriate alkoxide [Y⁺M⁻], where M is a cation, usually an alkali metal or alkaline earth metal cation.

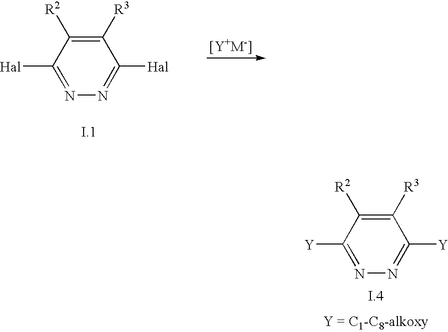

This reaction is usually carried out at temperatures of from 0° C. to 120° C., preferably from 25° C. to 100° C., in an inert organic solvent [cf. Helv. Chim. Acta, 37, 121-33; 1954; Chem. & Pharm. Bull., 13(5), 586-93; 1965].

Suitable solvents are aliphatic hydrocarbons, such as pentane, hexane, cyclohexane and petroleum ether, aromatic hydrocarbons, such as toluene, o-, m- and p-xylene, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide, particularly preferably alcohols, such as methanol, ethanol, n-propanol, isopropanol, n-butanol and tert-butanol, in particular methanol. It is also possible to use mixtures of the solvents mentioned.

The starting materials are generally reacted with one another in equimolar amounts. In terms of yield, it may be advantageous to employ an excess of Y⁺M⁻, based on I.1.

Compounds of the formula I in which one of the groups R¹ and R⁴ is cyano and the respective other group is halogen, in particular chlorine, or where both groups R¹ and R⁴ are cyano, correspond to the formulae I.5, I.6 and I.7, respectively. Depending on the chosen parameters, they can be obtained from compounds of the formula I.1 by reaction with cyanides.

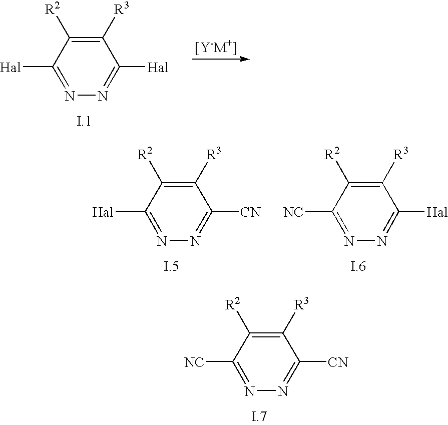

This reaction is usually carried out at temperatures of from 50° C. to 150° C., preferably from 80° C. to 100° C., in an inert organic solvent, if appropriate in the presence of a catalyst, such as p-toluenesulfonic acid sodium salt [cf. Heterocycles, 39(1), 345-56; 1994]. Usually, use is made of alkali metal or alkaline earth metal cyanides, preferably potassium cyanide.

Suitable solvents are aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide, particularly preferably dimethylformamide. It is also possible to use mixtures of the solvents mentioned.

The starting materials are generally reacted with one another in equimolar amounts. In terms of yield, it may be advantageous to employ an excess of the cyanide, based on I.1

Alternatively, the compounds of the formula I can be obtained by oxidation of 4,5-dihydropyridazines of the formula XII.

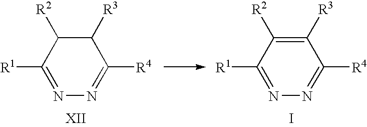

The oxidation is usually carried out at temperatures of from 20° C. to 100° C., preferably from 40° C. to 80° C., in an inert organic solvent in the presence of an oxidizing agent and/or a catalyst, such as, preferably, Pt or Pd, or oxides or peroxides, such as H₂O₂ [lit.: Comprehensive Organic Reactions, R. C. Larock, Chapter 5.1 Aromatization—Dehydrogenation, page 93, VCH, 1989; J Het, Chem, 26 (3), 717-719, 1989].

Reaction times are usually from 1 to 48 hours. Suitable solvents are aliphatic hydrocarbons, such as pentane, hexane, cyclohexane and petroleum ether, aromatic hydrocarbons, such as toluene, o-, m- and p-xylene, halogenated hydrocarbons, such as methylene chloride, chloroform and chlorobenzene, ethers, such as diethyl ether, diisopropyl ether, tert-butyl methyl ether, dioxane, anisole and tetrahydrofuran, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide. It is also possible to use mixtures of the solvents mentioned.

The oxidizing agent is usually employed in equimolar amounts, based on the compound of the formula XII. In terms of yield, it may be advantageous to employ the oxidizing agent in equimolar amounts or in an excess of up to 5 molar equivalents, based on XII.

Furthermore, dihydropyridazines may be obtained by chlorination or bromination and subsequent elimination of HCl or HBr. This reaction is usually carried out at temperatures of from −30° C. to 100° C., preferably from 0° C. to 80° C., in an inert organic solvent or in organic acids such as acetic acid [cf. lit. Synthesis, 1995, (10), 240-242; Syn Comm, 23(21), 2957-2964, 1993].

4,5-Dihydropyridazines of the formula XII can be obtained in an advantageous manner from 1,2-diketones of the formula XIII by reaction with hydrazine or hydrazine hydrate.

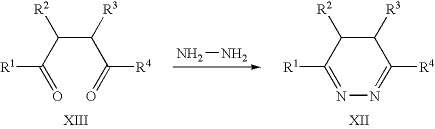

This reaction is usually carried out at temperatures of from −30° C. to 100° C., preferably from 0° C. to 80° C., in an inert organic solvent [cf. lit. Syn Comm, 31 (5), 645-651, 2001; Heterocycles, 57 (1), 39-46, 2002].

Suitable solvents are ethers, such as diethyl ether, diisopropyl ether, dimethoxyethane, tert-butyl methyl ether, dioxane, anisole and tetrahydrofuran, alcohols, such as methanol, ethanol, n-propanol, isopropanol, n-butanol and tert-butanol, particularly preferably alcohols. It is also possible to use mixtures of the solvents mentioned.

The starting materials are generally reacted with one another in equimolar amounts. In terms of yield, it may be advantageous to employ the hydrazine or hydrazine hydrate in an excess of up to 5 molar equivalents, based on XIII.

Diketones of the formula XIII can be obtained by reacting compounds of the formulae XIV and V, via intermediate XIIIa.

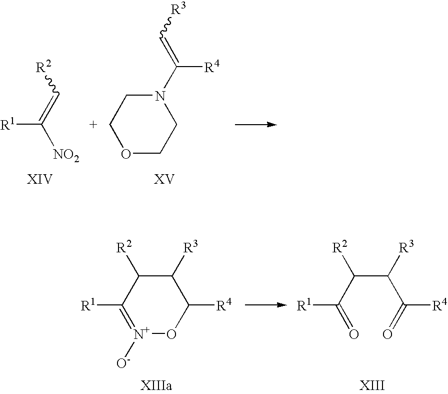

The reaction of the compounds XIV and XV is usually carried out at temperatures of from −30° C. to 100° C., preferably from 20° C. to 40° C., in the absence of a solvent or in an inert organic solvent [cf. lit. Tetrahedron, 45 (17), 5667-5678, 1989].

The reaction is preferably carried out in the absence of a solvent.

The reaction of the intermediate XIIIa is usually carried out at temperatures of from −30° C. to 80° C., preferably from 0° C. to 30° C., in an aqueous organic solvent, in an acidic medium, preferably at a pH of about 2 [cf. lit. Tetrahedron, 45 (17), 5667-5678, 1989; Tetrahedron, 45 (7), 2099-2108, 1989.

Suitable solvents are water and ethers, such as diethyl ether, diisopropyl ether, tert-butyl methyl ether, dioxane, anisole and tetrahydrofuran, alcohols, such as methanol, ethanol, n-propanol, isopropanol, n-butanol and tert-butanol, particularly preferably ethanol. It is also possible to use mixtures of the solvents mentioned.

Suitable for use as acids and acidic catalysts are inorganic acids, such as hydrofluoric acid, hydrochloric acid, hydrobromic acid, sulfuric acid and perchloric acid, Lewis acids, such as boron trifluoride, aluminum trichloride, iron(III) chloride, tin(IV) chloride, titanium(IV) chloride and zinc(II) chloride, and also organic acids, such as formic acid, acetic acid, propionic acid, oxalic acid, toluenesulfonic acid, benzenesulfonic acid, camphorsulfonic acid, citric acid and trifluoroacetic acid, preferably hydrochloric acid.

The acids are generally employed in catalytic amounts; however, they can also be employed in equimolar amounts, in excess or, if appropriate, as solvent.

Alternatively, the compounds of the formula I can be obtained from substituted hydrazonoketones of the formula XVI.

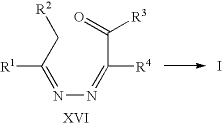

This reaction is usually carried out at temperatures of from 0° C. to 120° C., preferably from 50° C. to 100° C., in an inert organic solvent in the presence of a base [cf. lit. J Chem Res, 3, 187-189, 2005].

Suitable solvents are ethers, such as diethyl ether, diisopropyl ether, tert-butyl methyl ether, dimethoxyethane, dioxane, anisole and tetrahydrofuran, alcohols, such as methanol, ethanol, n-propanol, isopropanol, n-butanol and tert-butanol, particularly preferably ethanol. It is also possible to use mixtures of the solvents mentioned.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, sodium amide and potassium amide, alkali metal and alkaline earth metal carbonates, such as lithium carbonate, potassium carbonate and calcium carbonate, and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide and dimethoxymagnesium, moreover organic bases, for example tertiary amines, such as trimethylamine, triethylamine, diisopropylethylamine and N-methylpiperidine, pyridine, substituted pyridines, such as collidine, lutidine and 4-dimethylaminopyridine, and also bicyclic amines. Particular preference is given to tertiary amines, such as, for example, triethylamine.

The bases are generally employed in catalytic amounts; however, they can also be employed in equimolar amounts, in excess, preferably up to 2 molar equivalents, or, if appropriate, as solvent.

The hydrazonoketones of the formula XVI can be obtained by reacting compounds of the formulae XVII and XVIII.

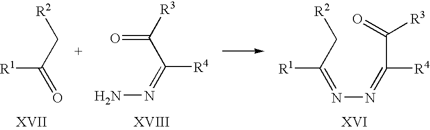

This reaction is usually carried out at temperatures of from 0° C. to 150° C., preferably from 20° C. to 120° C., in an inert organic solvent in the presence or absence of a base or an acidic catalyst [cf. lit. J für Prakt Chem, 328 (4), 551-557, 1986; J für Prakt Chem, 327 (1), 109-116, 1985; Synthesis, (5), 691-694, 2003].

Suitable solvents are aliphatic hydrocarbons, such as pentane, hexane, cyclohexane and petroleum ether, aromatic hydrocarbons, such as toluene, o-, m- and p-xylene, benzene, particularly preferably benzene, halogenated hydrocarbons, such as methylene chloride, chloroform and chlorobenzene, particularly preferably dichloromethane, ethers, such as diethyl ether, diisopropyl ether, tert-butyl methyl ether, dioxane, anisole and tetrahydrofuran, particularly preferably dioxane, nitriles, such as acetonitrile and propionitrile, ketones, such as acetone, methyl ethyl ketone, diethyl ketone and tert-butyl methyl ketone, alcohols, such as methanol, ethanol, n-propanol, isopropanol, n-butanol and tert-butanol, and also dimethyl sulfoxide, dimethylformamide and dimethylacetamide, particularly preferably dimethylformamide. It is also possible to use mixtures of the solvents mentioned.

Suitable for use as acids and acidic catalysts are inorganic acids, such as hydrofluoric acid, hydrochloric acid, hydrobromic acid, sulfuric acid and perchloric acid, Lewis acids, such as boron trifluoride, aluminum trichloride, iron(III) chloride, tin(IV) chloride, titanium(IV) chloride and zinc(II) chloride, and also organic acids, such as formic acid, acetic acid, propionic acid, oxalic acid, citric acid, trifluoroacetic acid and sulfonic acids, such as toluenesulfonic acid, benzenesulfonic acid, camphorsulfonic acid, particularly preferably p-toluenesulfonic acid. The acids are generally employed in catalytic amounts, preferably from 0.001 to 0.05 molar equivalents, based on XVIII; however, they can also be employed in equimolar amounts, in excess or, if appropriate, as solvent.

Suitable bases are, in general, inorganic compounds, such as alkali metal and alkaline earth metal hydroxides, such as lithium hydroxide, sodium hydroxide, potassium hydroxide and calcium hydroxide, alkali metal and alkaline earth metal oxides, such as lithium oxide, sodium oxide, calcium oxide and magnesium oxide, alkali metal and alkaline earth metal hydrides, such as lithium hydride, sodium hydride, potassium hydride and calcium hydride, alkali metal amides, such as lithium amide, sodium amide and potassium amide, alkali metal and alkaline earth metal carbonates, such as lithium carbonate, potassium carbonate and calcium carbonate, and also alkali metal bicarbonates, such as sodium bicarbonate, organometallic compounds, in particular alkali metal alkyls, such as methyllithium, butyllithium and phenyllithium, alkylmagnesium halides, such as methylmagnesium chloride, and also alkali metal and alkaline earth metal alkoxides, such as sodium methoxide, sodium ethoxide, potassium ethoxide, potassium tert-butoxide and dimethoxymagnesium, moreover organic bases, for example tertiary amines, such as trimethylamine, triethylamine, diisopropylethylamine and N-methylpiperidine, pyridine, substituted pyridines, such as collidine, lutidine and 4-dimethylaminopyridine, and also bicyclic amines. Particular preference is given to alkali metal bicarbonates, such as sodium bicarbonate.

The bases are generally employed in catalytic amounts; however, they can also be employed in equimolar amounts, in excess, preferably up to 2 molar equivalents, or, if appropriate, as solvent.

The starting materials are generally reacted with one another in equimolar amounts. In terms of yield, it may be advantageous to employ about 0.8 to 1.3 molar equivalents of XVII, based on XVIII.

The hydrazonoketones of the formula XVIII can be obtained by reacting diketones of the formula XIX with hydrazine or hydrazine hydrate.

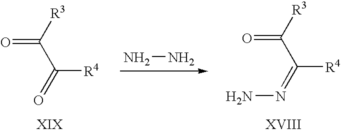

This reaction is usually carried out at temperatures of from −30° C. to 120° C., preferably from 0° C. to 80° C., in an inert organic solvent [cf. lit. J Med Chem, 48 (22), 6843-6854, 2005].

Suitable solvents are inter alia alcohols, such as methanol, ethanol, n-propanol, isopropanol, n-butanol and tert-butanol, particularly preferably ethanol. It is also possible to use mixtures of the solvents mentioned.

The starting materials are generally reacted with one another in equimolar amounts. In terms of yield, it may be advantageous to employ the hydrazine or hydrazine hydrate in a molar ratio of from 0.8 to 1.3, based on XIX.

Compounds of the formula I in which R¹ and/or R⁴ is/are alkyl can be obtained from the compounds I.1, preferably from the compounds I.1 in which both “Hal” are chlorine. Depending on the nature of the groups R² and R³ and the chosen reaction conditions and molar ratios of the reactants, the compounds I.8, I.9 and I.10 can be obtained with various selectivity by the following routes. In the formulae I.8, I.9 and I.10, R^{1′} and R^{4′} are alkyl, in particular methyl. Compounds of the formulae I.8, I.9 and I.10 can be obtained by reacting the compounds of the formula I.1 with alkylmagnesium halides.

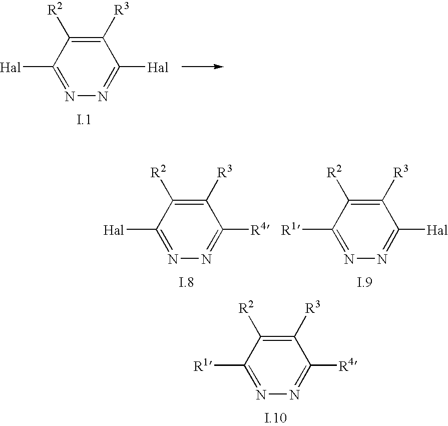

This reaction is usually carried out at temperatures of from −30° C. to 25° C., preferably from 0° C. to 20° C., in an inert organic solvent in the presence of a catalyst [cf. JACS, 124 (46), 13856-13863].

Suitable solvents are ethers, such as diethyl ether, diisopropyl ether, tert-butyl methyl ether, dioxane, anisole and tetrahydrofuran, and also N-methylpyrrolidone (NMP), particularly preferably THF and NMP.

Advantageous for use as catalyst are Fe (acac)₃ or Fe(salen)Cl in catalytic amounts, such as, for example, 5 mol %.

The starting materials are generally reacted with one another in equimolar amounts. In terms of yield, it may be advantageous to employ a slight excess of alkylmagnesium halide, based on the pyridazine.

Alternatively, the compounds of the formulae I.8, I.9 and I.10 can also be obtained by reacting the compounds of the formula I.1 with alkylzinc halides at temperatures of from 0° C. to 120° C., preferably from 20° C. to 60° C., in an inert organic solvent in the presence of a catalyst [cf. Tetrahedron Letters, 46 (8), 1303-1305 (2005)].

Suitable solvents are ethers, such as diethyl ether, diisopropyl ether, tert-butyl methyl ether, dioxane, anisole and tetrahydrofuran.

The catalyst used is advantageously a Pd(0) catalyst, particularly preferably Pd(PPh₃)₄, in an amount of about 5 mol %.

The starting materials are generally reacted with one another in equimolar amounts in order to obtain the monosubstituted products. If the zinc reagent is employed in larger amounts (1.6 eq.), some disubstituted product is obtained as byproduct. If the reaction temperature is increased from 20° to 60° C., mainly disubstituted product is obtained.

Alternatively, the group L¹ may be introduced at the stage of pyridazines of the formula XIX by nucleophilic substitution according to the synthesis shown below.

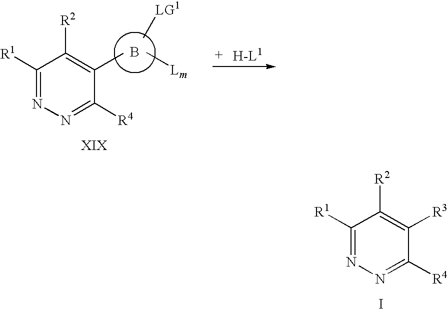

In formula XIX, the variables have the meanings mentioned above, LG¹ is a nucleophilically replaceable group, such as halogen, for example fluorine, and [symbol] is phenyl or a 5- or 6-membered heteroaromatic group which contains 1, 2, 3 or 4 heteroatoms selected from the group consisting of O, S and N as ring members.

The reaction of XIX with H-L¹ is carried out, for example, according to the method described in WO 2005/030775 and is advantageously carried out in the presence of strong bases. Suitable bases are, for example, alkali metal hydroxides, such as sodium hydroxide or potassium hydroxide, alkali metal carbonates, such as sodium carbonate or potassium carbonate, alkaline earth metal carbonates, such as calcium carbonate or magnesium carbonate, or alkali metal hydrides, such as lithium hydride or sodium hydride. The reaction can be carried out in the presence of a solvent. Suitable solvents are aprotic solvents, for example N,N-disubstituted amides, such as N,N-dimethylformamide, N,N-dimethylacetamide or N-methylpyrrolidone, sulfoxides, such as dimethyl sulfoxide, or ethers, such as diethyl ether, diisopropyl ether, tert-butyl ether, 1,2-dimethoxyethane, tetrahydrofuran, dioxane or anisole. The reaction is usually carried out at temperatures in the range of from 0° C. to the boiling point of the solvent.

If T in group L¹ is OH or a primary or secondary amino group, it is advantageous to protect the hydroxyl group or the amino group. A suitable protective group for the hydroxyl group is, for example, the benzyl group, which optionally carries a methoxy group in the 4-position of the phenyl ring. The protective group for the hydroxyl group can be removed, for example, by catalytic hydrogenolysis or with the aid of 2,3-dichloro-5,6-dicyano-1,4-benzoquinone (DDQ). A suitable protective group for primary and secondary amino groups is, for example, the tert-butoxycarbonyl group (Boc), which is usually removed using trifluoroacetic acid or p-toluenesulfonic acid.

Pyridazines of the formula XIX can be prepared analogously to the synthesis routes above by modifying the precursors with respect to the nature of R³.

Compounds H-L¹ are generally commercially available or can be prepared by processes known from the literature.

Alternatively, compounds of the formula I in which L¹ is a group attached via oxygen can be obtained according to the process described below.

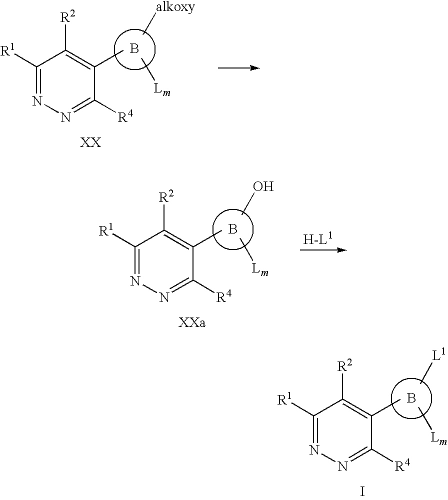

In the formulae XX and XXa, the variables and [symbol] have the meanings mentioned above.

In a first step, the compound XX is reacted with a Lewis acid, such as aluminum trichloride or iron(III) chloride, giving the phenolic compound XXa. Usually, the ether cleavage is carried out in an organic solvent, for example in an aromatic hydrocarbon, such as benzene, toluene or xylene. The introduction of the group L¹ is carried out by nucleophilic substitution of the hydroxyl group under basic conditions, as described above.

Pyridazines of the formula XX can be prepared analogously to the synthesis routes above by modifying the precursors with respect to the nature of R³.

Compounds of the formula I in which L¹ is a group attached via carbon can advantageously be prepared from compounds XXa. Initially, the hydroxyl compound XXa is reacted with trifluoromethanesulfonic anhydride giving a trifluoromethanesulfonate XXb, which is then treated with an aminoalkylboronic acid) XXI;

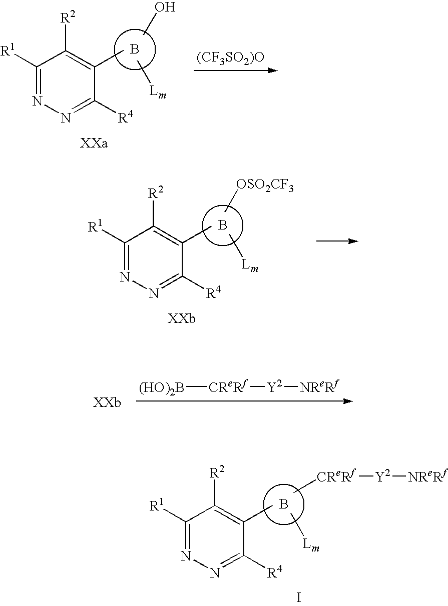

In the formulae XXb and XXI, the variables and [symbol] have the meanings mentioned above.

Compounds of the formula I in which L¹ is a group attached via nitrogen can advantageously be prepared from precursors in which group [symbol] carries an amino group, which, if appropriate, can be obtained from the corresponding nitro-substituted compounds by reduction.

The required starting materials are known from the literature or can be prepared in accordance with the literature cited.

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which are purified or freed from volatile components under reduced pressure and at moderately elevated temperature. If the intermediates and end products are obtained as solids, purification can also be carried out by recrystallization or digestion.

If individual compounds of the formula I can not be obtained by the routes described above, they can be prepared by derivatization of other compounds I.

If the synthesis yields mixtures of isomers, a separation is generally not necessarily required since in some cases the individual isomers can be interconverted during preparation for application or during application (for example under the action of light, acid or bases). Such conversions may also take place after application, for example in the case of the treatment of plants in the treated plants or in the harmful fungus to be controlled.

In the definitions of the symbols given in the above formulae, collective terms were used which are generally representative for the following substituents:

halogen: fluorine, chlorine, bromine and iodine;

alkyl: saturated straight-chain or branched hydrocarbon radicals having 1 to 4, 6 or 8 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;

haloalkyl: straight-chain or branched alkyl groups having 1 to 2, 4 or 6 carbon atoms (as mentioned above), where some or all of the hydrogen atoms in these groups may be replaced by halogen atoms as mentioned above: in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;

alkenyl: unsaturated straight-chain or branched hydrocarbon radicals having 2 to 4, 6 or 8 carbon atoms and one or two double bonds in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;

alkynyl: straight-chain or branched hydrocarbon groups having 2 to 4, 6 or 8 carbon atoms and one or two triple bonds in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;

cycloalkyl: mono- or bicyclic saturated hydrocarbon groups having 3 to 6 or 8 carbon ring members, for example C₃-C₈-cycloalkyl, such as cyclopropyl, cyclobutyl, cyclopentyl, cyclohexyl, cycloheptyl and cyclooctyl;

aryl: a mono-, bi- or tricyclic aromatic hydrocarbon group which contains 6, 8, 10, 12 or 14 ring members, such as phenyl, naphthyl or anthracenyl, preferably phenyl or naphthyl, in particular phenyl;

a five- or six-membered saturated, partially unsaturated or aromatic heterocycle which contains one, two, three or four heteroatoms from the group consisting of O, N and S:
non-aromatic saturated or partially unsaturated 5- or 6-membered heterocyclyl which contains one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 2-pyrrolin-2-yl, 2-pyrrolin-3-yl, 3-pyrrolin-2-yl, 3-pyrrolin-3-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl and 2-piperazinyl;
5-membered heteroaryl which contains one to four nitrogen atoms or one to three nitrogen atoms and one sulfur or oxygen atom: 5-membered heteroaryl groups which, in addition to carbon atoms, may contain one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, and 1,3,4-triazol-2-yl;
6-membered heteroaryl which contains one to three or one to four nitrogen atoms: 6-membered heteroaryl groups which, in addition to carbon atoms, may contain one to three or one to four nitrogen atoms as ring members, for example 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl;

alkylene: divalent unbranched chains of 2 to 8 CH₂ groups, for example CH₂CH₂, CH₂CH₂CH₂, CH₂CH₂CH₂CH₂, CH₂CH₂CH₂CH₂CH₂, CH₂CH₂CH₂CH₂CH₂CH₂, CH₂CH₂CH₂CH₂CH₂CH₂CH₂ and CH₂CH₂CH₂CH₂CH₂CH₂CH₂CH₂;

oxyalkylene: divalent unbranched chains of 2 to 4 CH₂ groups where one valency is attached via an oxygen atom to the skeleton, for example OCH₂CH₂, OCH₂CH₂CH₂ and OCH₂CH₂CH₂CH₂;

oxyalkyleneoxy: divalent unbranched chains of 1 to 3 CH₂ groups where both valencies are attached via an oxygen atom to the skeleton, for example OCH₂O, OCH₂CH₂O and OCH₂CH₂CH₂O.

In accordance with the present invention, agriculturally acceptable salts are in particular the salts of those cations and the acid addition salts of those acids whose cations and anions, respectively, have no adverse effect on the pesticidal action of the pyrimidines according to the invention.

Agriculturally useful salts include in particular the salts of those cations or the acid addition salts of those acids whose cations and anions, respectively, have no adverse effect on the fungicidal action of the compounds I. Thus, suitable cations are in particular the ions of the alkali metals, preferably sodium and potassium, of the alkaline earth metals, preferably calcium, magnesium and barium, and of the transition metals, preferably manganese, copper, zinc and iron, and also the ammonium ion which, if desired, may carry from one to four (C₁-C₄)-alkyl substituents and/or one phenyl or benzyl substituent, preferably diisopropylammonium, tetramethylammonium, tetrabutylammonium, trimethylbenzylammonium, and also phosphonium ions, sulfonium ions, preferably tri(C₁-C₄)-alkylsulfonium, and sulfoxonium ions, preferably tri(C₁-C₄)-alkylsulfoxonium.

Anions of useful acid addition salts are, primarily, chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and also the anions of (C₁-C₄)-alkanoic acids, preferably formate, acetate, propionate and butyrate. They can be formed by reacting I with an acid of the corresponding anion, preferably hydrochloric acid, hydrobromic acid, sulfuric acid, phosphoric acid or nitric acid.

Suitable pharmaceutically acceptable salts are especially physiologically tolerated salts of the compound I, in particular the acid addition salts with physiologically acceptable acids. Examples of suitable organic and inorganic acids are hydrochloric acid, hydrobromic acid, phosphoric acid, sulfuric acid, C₁-C₄-alkylsulfonic acids, such as methanesulfonic acid, aromatic sulfonic acids, such as benzenesulfonic acid and toluenesulfonic acid, oxalic acid, maleic acid, fumaric acid, lactic acid, tartaric acid, adipic acid and benzoic acid. Further suitable acids are described, for example, in Fortschritte der Arzneimittelforschung, Volume 10, pages 224 ff., Birkhäuser Verlag, Basle and Stuttgart, 1966, the entire contents of which is expressly incorporated herein by way of reference.

The scope of the present invention includes the (R)- and (S)-isomers and the racemates of compounds of the formula I having chiral centers.

As a result of hindered rotation of asymmetrically substituted groups, atrope isomers of compounds of the formula I may be present. They also form part of the subject matter of the invention.

The embodiments of the intermediates with respect to the variables correspond to those of the formula I.

With a view to the intended use of the pyridazines of the formula I, particular preference is given to the following meanings of the substituents, in each case on their own or in combination:

One aspect relates to compounds I in which R¹ and R⁴ are halogen, in particular chlorine. These compounds correspond to the formula I.1.

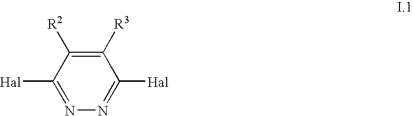

A further aspect relates to compounds I in which R¹ is halogen, in particular chlorine, and R⁴ is C₁-C₅-alkoxy, such as methoxy, ethoxy, n-propoxy, isopropoxy, in particular methoxy. These compounds correspond to the formula I.2.

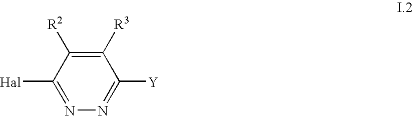

A further aspect relates to compounds I in which R¹ is C₁-C₆-alkoxy, such as methoxy, ethoxy, n-propoxy, isopropoxy, in particular methoxy, and R⁴ is halogen, in particular chlorine. These compounds correspond to the formula I.3.

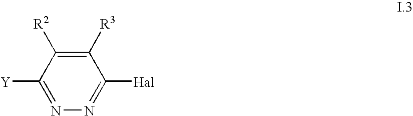

A further aspect relates to compounds I in which R¹ and R⁴ are C₁-C₆-alkoxy, such as methoxy, ethoxy, n-propoxy, isopropoxy, in particular methoxy. These compounds correspond to the formula I.4.

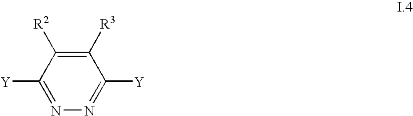

A further aspect relates to compounds I in which R¹ is halogen, in particular chlorine, and R⁴ is cyano. These compounds correspond to the formula I.5.

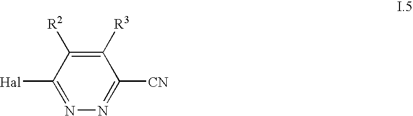

A further aspect relates to compounds I in which R¹ is cyano and R⁴ is halogen, in particular chlorine. These compounds correspond to the formula I.6.

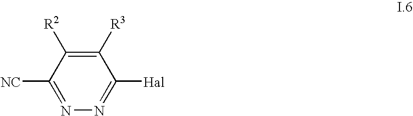

A further aspect relates to compounds I in which R¹ and R⁴ are cyano. These compounds correspond to the formula I.7.

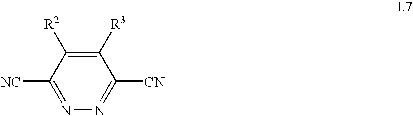

A further aspect relates to compounds I in which R¹ is halogen, in particular chlorine, and R⁴ is alkyl, in particular methyl. These compounds correspond to the formula I.8 in which R^{4′} has the meaning given above.

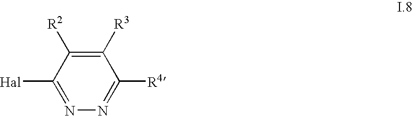

A further aspect relates to compounds I in which R¹ is alkyl, in particular methyl, and R⁴ is halogen, in particular chlorine. These compounds correspond to the formula I.9, in which R^{1′} has the meaning given above.

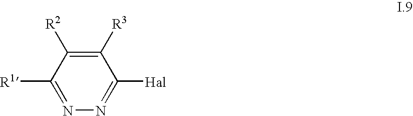

A further aspect relates to compounds I in which R¹ and R⁴ are alkyl, in particular methyl. These compounds correspond to the formula I.10 in which R^{1′} and R^{4′} have the meaning given above.

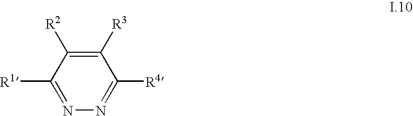

In the compounds according to the invention, R² is preferably C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₂-cycloalkyl (including in particular C₃-C₅-cycloalkyl and/or C₉-C₁₂-cycloalkyl), C₃-C₁₂-halocycloalkyl, C₃-C₁₂-cycloalkenyl, C₃-C₁₂-halocycloalkenyl, naphthyl or halonaphthyl or a five-, six-, seven-, eight-, nine- or ten-membered saturated, partially unsaturated or aromatic heterocycle which is attached via carbon and contains one, two, three or four heteroatoms from the group consisting of oxygen, nitrogen and sulfur, more preferably C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₂-cycloalkyl, C₃-C₁₂-halocycloalkyl, C₃-C₁₂-cycloalkenyl or C₃-C₁₂-halocycloalkenyl or a five-, six-, seven-, eight-, nine- or ten-membered saturated, partially unsaturated or aromatic heterocycle which is attached via carbon and contains one, two, three or four heteroatoms from the group consisting of oxygen, nitrogen and sulfur; where R² may contain one, two, three or four identical or different groups R^{a}, as defined herein.

One aspect relates to compounds I in which R² is an aliphatic group which is unsubstituted or substituted by R^{a}, as defined at the outset. These compounds correspond to formula I.a.

One aspect of the compounds I.a relates to those compounds in which R² is C₁-C₁₀-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₁₂-cycloalkyl, C₃-C₁₂-halocycloalkyl, C₃-C₁₂-cycloalkenyl, C₃-C₁₂-halocycloalkenyl.

One aspect relates to compounds I.a in which R² is C₁-C₈-alkyl, in particular branched C₃-C₈-alkyl, C₁-C₆-haloalkyl, C₃-C₈-alkenyl, in particular branched C₃-C₅-alkenyl, C₃-C₆-cycloalkyl which may have a C₁-C₄-alkyl group, or C₅-C₆-cycloalkenyl which may have a C₁-C₄-alkyl group.

A further aspect relates to compounds I.a in which R² is C₃-C₁₂-cycloalkyl, in particular C₆-C₈-cycloalkyl.

A further aspect relates to compounds I.a in which R² is C₁-C₁₀-alkyl, in particular C₃-C₈-alkyl, which is optionally substituted by one, two or three R^{a}. Here, R^{a} is preferably selected from the group consisting of halogen, cyano, C₁-C₆-alkyl, C₂-C₆-alkenyl, C₂-C₆-alkynyl, C₁-C₆-alkoxycarbonyl, C₁-C₆-alkoximino, C₂-C₆-alkenyloximino, C₂-C₆-alkynyloximino, C₃-C₆-cycloalkyl or C₅-C₆-cycloalkenyl, where the aliphatic and/or alicyclic groups for their part may be substituted by one, two or three groups R^{b}. Here, each R^{b} is preferably independently halogen, cyano, C₁-C₈-alkyl, C₂-C₆-alkenyl, C₂-C₆-alkynyl, C₁-C₆-alkoxy, C₁-C₆-alkylcarbonyl or C₁-C₆-haloalkylcarbonyl.

According to one aspect of this embodiment, R² is C₁-C₁₀-haloalkyl, in particular C₃-C₈-haloalkyl.

A further aspect relates to compounds I.a in which R² is C₂-C₁₀-alkenyl, in particular C₃-C₈-alkenyl, which is optionally substituted by one, two or three R^{a}, as defined herein.

A further aspect relates to compounds I.a in which R² is C₂-C₁₀-alkynyl, in particular C₃-C₈-alkynyl, which is optionally substituted by one, two or three R^{a}, as defined herein.

A further aspect relates to compounds I.a in which R² is C₃-C₁₂-cycloalkenyl, in particular C₈-C₁₀-cycloalkenyl, especially C₅- or C₆-cycloalkenyl, which is optionally substituted by one, two or three R^{a}, as defined herein. According to one aspect of this embodiment according to the invention, the cycloalkenyl group is mono-, di- or trisubstituted by C₁-C₄-alkyl, such as, for example, methyl and/or ethyl.

One aspect relates to compounds I in which R² is a five-, six-, seven-, eight-, nine- or ten-membered saturated, partially unsaturated or aromatic heterocycle which is attached via carbon to the pyridazine skeleton and which contains one, two, three or four heteroatoms from the group consisting of oxygen, nitrogen and sulfur, where the heterocycle is unsubstituted or substituted by one, two, three or four identical or different substituents R^{a}, as defined herein. These compounds correspond to the formula I.B. According to a preferred aspect of this embodiment, R² is an optionally substituted five- or six-membered saturated or aromatic heterocycle which is attached via carbon to the pyridazine skeleton.

If R² carries one, two, three or four, preferably one, two or three, identical or different groups R^{a}, then R^{a} is preferably selected from the group consisting of halogen, cyano, C₁-C₆-alkyl, C₂-C₆-alkenyl, C₂-C₆-alkynyl, C₁-C₆-alkoxy, C₁-C₆-alkoxycarbonyl, C₁-C₆-alkoximino, C₂-C₆-alkenyloximino, C₂-C₆-alkynyloximino, C₃-C₆-cycloalkyl, C₅-C₆-cycloalkenyl, where the aliphatic or alicyclic groups for their part may be partially or fully halogenated or may carry one, two or three groups R^{b}.

If R^{a} carries at least one group R^{b}, then R^{b} is preferably selected from the group consisting of halogen, cyano, C₁-C₆-alkyl, C₁-C₆-haloalkyl, C₂-C₆-alkenyl, C₂-C₆-alkynyl, C₁-C₆-alkylcarbonyl, C₁-C₆-haloalkylcarbonyl and C₁-C₆-alkoxy.

According to a preferred embodiment of the invention, R² is C₁-C₁₀-alkyl, in particular C₃-C₅-alkyl, which is optionally substituted by one, two or three R^{a}. Here, R^{a} is preferably selected from the group consisting of halogen, cyano, C₁-C₈-alkyl, C₂-C₆-alkenyl, C₂-C₆-alkynyl, C₁-C₆-alkoxycarbonyl, C₁-C₆-alkoximino, C₂-C₆-alkenyloximino, C₂-C₆-alkynyloximino, C₃-C₆-cycloalkyl and C₅-C₆-cycloalkenyl, where the aliphatic and/or alicyclic groups for their part may be substituted by one, two or three groups R^{b}. Here, each R^{b} is preferably independently chosen from the group consisting of halogen, cyano, C₁-C₈-alkyl, C₂-C₆-alkenyl, C₂-C₆-alkynyl, C₁-C₆-alkoxy, C₁-C₆-alkylcarbonyl and C₁-C₆-haloalkylcarbonyl. According to one aspect of this embodiment, R² is C₁-C₁₀-haloalkyl, in particular C₃-C₈-haloalkyl.

A further aspect relates to compounds I in which R² is an aryl group which is unsubstituted or substituted by R^{a}, as defined at the outset, such as phenyl or naphthyl, in particular a phenyl group. These compounds correspond to formula I.b. Preferred compounds of the formula I.b have a group selected from the group consisting of phenyl, 3-fluorophenyl, 4-fluorophenyl, 3-chlorophenyl, 4-chlorophenyl, 3-methoxyphenyl, 4-methoxyphenyl, 3-hydroxyphenyl, 4-hydroxyphenyl, 3-tolyl and 4-tolyl in position R²

A further aspect relates to compounds I in which R² is a heteroaryl group which is attached via carbon and which is unsubstituted or substituted by R^{a}, as defined at the outset. These compounds correspond to formula I.c. Preferred compounds of the formula I.c have a group selected from the group consisting of pyridin-3-yl and pyridin-4-yl in position R².

A further aspect relates to compounds I in which R² is a heteroaryl group which is attached via nitrogen and which is unsubstituted or substituted by R^{a}, as defined at the outset. These compounds correspond to formula I.d.

According to one embodiment, R² is not optionally substituted phenyl.

According to a further embodiment, R² is not C₆-C₈-cycloalkyl, more preferably not C₃-C₁₂-cycloalkyl. Preferably, R² is not C₆-C₈-cycloalkyl, more preferably not C₃-C₁₂-cycloalkyl.

According to a further preferred embodiment of the invention, R² is C₂-C₁₀-alkenyl, in particular C₃-C₈-alkenyl, which is optionally substituted by one, two or three R^{a}, as defined herein.

According to a further preferred embodiment of the invention, R² is C₂-C₁₀-alkynyl, in particular C₃-C₈-alkynyl, which is optionally substituted by one, two or three R^{a}, as defined herein.

According to a further preferred embodiment of the invention, R² is C₃-C₁₂-cycloalkenyl, in particular C₅-C₁₀-cycloalkenyl, especially C₅- or C₆-cycloalkenyl, which is optionally substituted by one, two or three R^{a}, as defined herein. According to one aspect of this embodiment according to the invention, the cycloalkenyl group is mono-, di- or trisubstituted by C₁-C₄-alkyl, such as, for example, methyl and/or ethyl.

According to a further preferred embodiment of the invention, R² is a five-, six-, seven-, eight-, nine- or ten-membered saturated, partially unsaturated or aromatic heterocycle which is attached via carbon to the pyridazine skeleton and which contains one, two, three or four heteroatoms from the group consisting of oxygen, nitrogen and sulfur, where the heterocycle is unsubstituted or substituted by one, two, three or four identical or different substituents R^{a}, as defined herein.

A further aspect relates to compounds I in which R³ is an aryl group which is unsubstituted or substituted by R^{a} as defined at the outset, such as phenyl or naphthyl, in particular a phenyl group. These compounds correspond to formula I.A.

In one aspect of the compounds I.A, R³ is phenyl which, in addition to a group L¹ in the ortho-position to the bond to the pyridazine skeleton, carries at least one further group Lₘ.

In a further aspect of the compounds I.A, R³ is phenyl which is substituted only by one group L¹ in the ortho-position to the bond to the pyridazine skeleton.

In a further aspect of the compounds I.A, R³ is phenyl which, in addition to two groups L¹ in the two ortho-positions to the bond to the pyridazine skeleton, carries at least one further group Lₘ.

In a further aspect of the compounds I.A, R³ is phenyl which is substituted only by two groups L¹ in the two ortho-positions to the bond to the pyridazine skeleton.

A further aspect relates to compounds I in which R³ is a heteroaryl group which is attached via carbon and which is unsubstituted or, as defined at the outset, substituted by R^{a}. These compounds correspond to formula I.B.

A further aspect relates to compounds I in which R³ is a heteroaryl group which is attached via nitrogen and which is unsubstituted or, as defined at the outset, substituted by R^{a}. These compounds correspond to formula I.C.

In a further preferred embodiment of the invention, R³ is phenyl, pyridinyl, for example 2-, 3- or 4-pyridinyl, pyrimidinyl, for example 2-, 4- or 5-pyrimidinyl, pyrazinyl, for example 2-pyrazinyl, pyridazinyl, for example 3- or 4-pyridazinyl, triazinyl, furyl, for example 2- or 3-furyl, thienyl, for example 2- or 3-thienyl, pyrrolyl, for example 2- or 3-pyrrolyl, pyrazolyl, for example 1-, 3-, 4- or 5-pyrazolyl, imidazolyl, for example 1-, 2-, 4- or 5-imidazolyl, oxazolyl, for example 2-, 4- or 5-oxazolyl, isoxazolyl, for example 3-, 4- or 5-isoxazolyl, thiazolyl, for example 2-, 4- or 5-thiazolyl, isothiazolyl, for example 3-, 4- or 5-isothiazolyl, triazolyl, for example 1-, 4- or 5-[1,2,3]-1H-triazolyl, 2-, 4- or 5-[1,2,3]-2H-triazolyl, 1-, 3- or 5-[1,2,4]-1H-triazolyl or 3-, 4- or 5-[1,2,4′-4H-triazolyl, oxadiazolyl, for example 4- or 5-[1,2,3]-oxadiazolyl, 3- or 5-[1,2,4]-oxadiazolyl or 2- or 5-[1,3,4]-oxadiazolyl, thiadiazolyl, for example 4- or 5-[1,2,3]-thiadiazolyl, 3- or 5-[1,2,4]-thiadiazolyl or 2- or 5-[1,3,4]-thiadiazolyl, or tetrazolyl, for example 1-, 2- or 5-[1,2,3,4]tetrazolyl, which carries one substituent L¹ and 0, 1, 2, 3 or 4, preferably 0, 1 or 2, substituents L, where L¹ and L² are defined as described above or preferably as described below.

Particularly preferably, R³ is phenyl, pyridinyl, for example 2-, 3- or 4-pyridinyl, pyrimidinyl, especially 4- or 5-pyrimidinyl, pyrazinyl, for example 2-pyrazinyl, pyridazinyl, for example 3- or 4-pyridazinyl, furyl, for example 2- or 3-furyl, thienyl, for example 2- or 3-thienyl, pyrazolyl, especially 1- or 5-pyrazolyl, imidazolyl, especially 1-, 2- or 5-imidazolyl, oxazolyl, for example 2-, 4- or 5-oxazolyl, isoxazolyl, for example 3-, 4- or 5-isoxazolyl, thiazolyl, for example 2-, 4- or 5-thiazolyl, isothiazolyl, for example 3-, 4- or 5-isothiazolyl, or triazolyl, especially 1-[1,2,4]-1H-triazolyl which carries one substituent L¹ and 0, 1, 2, 3 or 4, preferably 0, 1 or 2, in particular 1 or 2, substituents L, where L¹ and L are as defined above or preferably as described below.

In a preferred embodiment of the invention, R³ is phenyl which is substituted by a group L¹ and 0, 1, 2, 3 or 4 radicals L.

Suitable groups L are in particular the following groups: halogen, such as fluorine or chlorine; cyano; nitro; alkoxycarbonyl; aminocarbonyl; C₁-C₄-alkyl, such as methyl; C₁-C₄-haloalkyl, such as trifluoromethyl; C₁-C₄-alkoxy, such as methoxy.

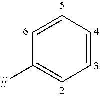

Aspects of R³ relate in particular to phenyl groups which, in addition to the group L¹, may have the following substituents:

position 2: fluorine, chlorine, methyl; position 3: hydrogen, fluorine, methoxy; position 4: hydrogen, fluorine, chlorine, methyl, methoxy, cyano, nitro, alkoxycarbonyl, aminocarbonyl, haloalkyl, particularly preferably fluorine, chlorine, methyl, methoxy, cyano; position 5: hydrogen, fluorine, chlorine, methyl; particularly preferably hydrogen, fluorine; position 6: hydrogen, fluorine, chlorine, methyl; particularly preferably hydrogen, fluorine.

The group L¹ is preferably located in position 3, 4 or 5.

In a preferred embodiment of the invention, R³ is one of the groups A1 or A2 (m=0, 1, 2, 3, 4).

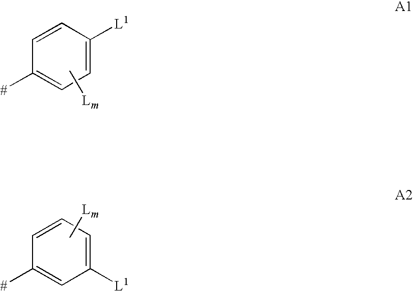

Here, Lₘ is preferably one of the substituent combinations below:

2-Cl; 2-F; 2-CH₃; 2,6-F₂; 2,6-Cl₂; 2-F, 6-CH₃; 2,4,6-F₃; 2,6-F₂-4-OCH₃; 2-Cl-4-OCH₃; 2-CH₃-4-F; 2-CF₃; 2-OCH₃,6-F; 2,4-F2; 2-F-4-Cl; 2-F-6-Cl; 2-Cl,4-F; 2-Cl,5-F; 2,3-F₂; 2,5-F₂; 2,3,4-F₃; 2-CH₃; 2,4-(CH₃)₂; 2-CH₃-4-Cl; 2-CH₃,5-F; 2-F,4-CH₃; 2,6-(CH₃)₂; 2,4,6-(CH₃)₃; 2,6-F2,4-CH₃. Particularly preferably, Lₘ is one of the substituent combinations below: 2-F; 2-Cl; 2-CH₃; 2,6-F₂; 2-F,6-Cl; 2-F,6-CH₃.

The compounds of the formula I which carry group A1 or A2 correspond to the formulae I.A1 and I.A2, respectively (m=0, 1, 2, 3, 4).

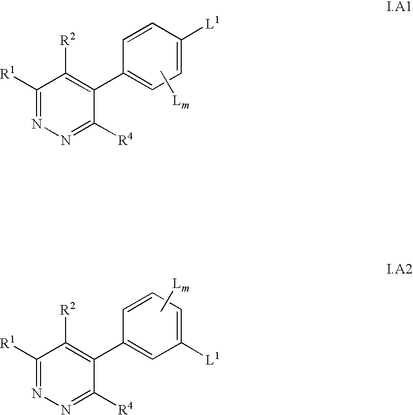

In a further embodiment of the invention, R³ is a 5-membered heteroaryl which is substituted by L¹ and, if appropriate, by 1, 2 or 3 groups L. Here, the 5-membered heteroaryl ring is preferably selected from the group consisting of thienyl, for example 2- or 3-thienyl, pyrazolyl, for example 1-, 3-, 4- or 5-pyrazolyl, and thiazolyl, for example 2-, 4- or 5-thiazolyl.

In a further embodiment of the invention, R³ is a 6-membered heteroaryl which is substituted by a group L¹ and, if appropriate, by 1, 2 or 3 groups L and which contains one to three nitrogen atoms. Here, the 6-membered heteroaryl ring is preferably selected from the group consisting of pyridinyl, for example 2-, 3- or 4-pyridinyl, pyrimidinyl, for example 2-, 4- or 5-pyrimidinyl, pyrazinyl, for example 2-pyrazinyl and pyridazinyl, for example 3- or 4-pyridazinyl.

In a preferred embodiment of the invention, R³ is pyridyl which is attached in the 2-, 3- or 4-position to the pyridazine ring and which may carry 1, 2 or 3 identical or different substituents L, which are preferably selected from the group consisting of fluorine, chlorine, bromine, cyano, nitro, methyl, ethyl, methoxy, methylthio, hydroximinomethyl, hydroximinoethyl, methoximinomethyl, methoximinoethyl and trifluoromethyl. A preferred aspect of such compounds are those of the formulae I.B1 and I.B2 (m=0, 1, 2, 3).

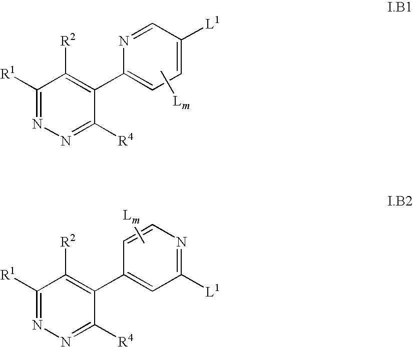

In an alternative preferred embodiment of the invention, R³ is pyrimidyl which is attached in the 2- or 4-position to the pyridazine ring and which may carry 1 or 2 identical or different substituents L, which are preferably selected from the group consisting of fluorine, chlorine, bromine, cyano, nitro, methyl, ethyl, methoxy, methylthio, hydroximinomethyl, hydroximinoethyl, methoximinomethyl, methoximinoethyl and trifluoromethyl. A preferred aspect of such compounds are those of the formulae I.B3 and I.B4 (m=0, 1, 2).

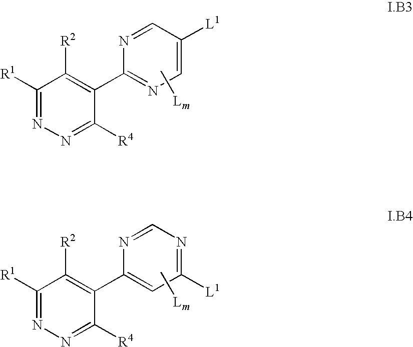

In an alternative preferred embodiment of the invention, R³ is thienyl which is attached in the 2- or 3-position to the pyridazine ring and which may carry 1 or 2 identical or different substituents L, which are preferably selected from the group consisting of fluorine, chlorine, bromine, cyano, nitro, methyl, ethyl, methoxy, methylthio, hydroximinomethyl, hydroximinoethyl, methoximinomethyl, methoximinoethyl and trifluoromethyl. A preferred aspect of such compounds are those of the formulae I.B5 and I.B6 (m=0, 1, 2).

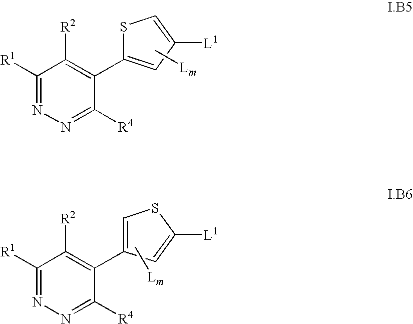

In an alternative preferred embodiment of the invention, R³ is thiazolyl which is attached in the 2-, 4- or 5-position to the pyridazine ring and which may carry one substituent L, which is preferably selected from the group consisting of fluorine, chlorine, bromine, cyano, nitro, methyl, ethyl, methoxy, methylthio, hydroximinomethyl, hydroximinoethyl, methoximinomethyl, methoximinoethyl and trifluoromethyl. A preferred aspect of such compounds are those of the formulae I.B7 and I.B8 (m=0, 1).

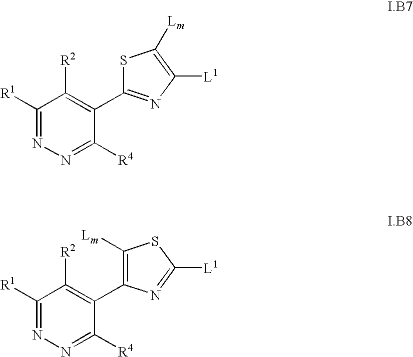

In an alternative preferred embodiment of the invention, R³ is imidazolyl which is attached in the 4- or 5-position to the pyridazine ring and which may carry 1 or 2 identical or different substituents L, which are preferably selected from the group consisting of fluorine, chlorine, bromine, cyano, nitro, methyl, ethyl, methoxy, methylthio, hydroximinomethyl, hydroximinoethyl, methoximinomethyl, methoximinoethyl and trifluoromethyl. A preferred aspect of such compounds are those of the formulae I.B9 and I.B10 (m=0, 1).

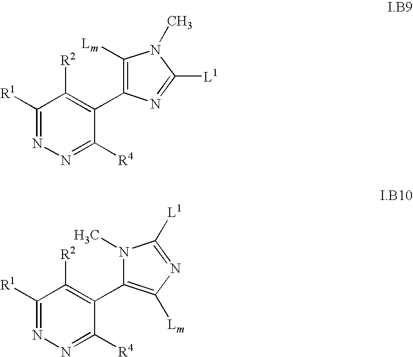

In an alternative preferred embodiment of the invention, R³ is pyrazolyl which is attached in the 1-, 3-, 4- or 5-position to the pyridazine ring and which may carry 1 or 2 identical or different substituents L, which are preferably selected from the group consisting of fluorine, chlorine, bromine, cyano, nitro, methyl, ethyl, methoxy, methylthio, hydroximinomethyl, hydroximinoethyl, methoximinomethyl, methoximinoethyl and trifluoromethyl. A preferred aspect of such compounds are those of the formulae I.B11, I.B12 and I.B13 (m=0, 1).

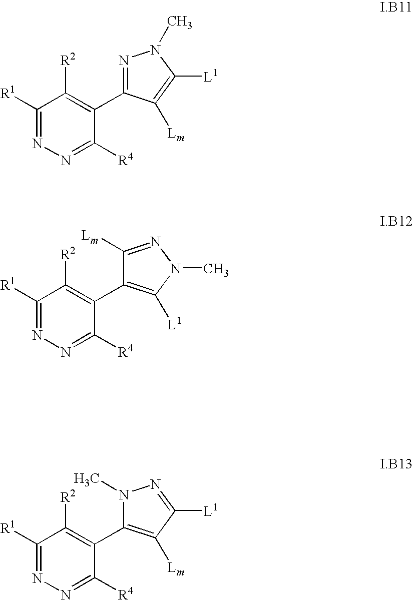

In an alternative preferred embodiment of the invention, R³ is oxazolyl which is attached in the 2-, 3- or 4-position to the pyridazine ring and which may carry one substituent L, which is preferably selected from the group consisting of fluorine, chlorine, bromine, cyano, nitro, methyl, ethyl, methoxy, methylthio, hydroximinomethyl, hydroximinoethyl, methoximinomethyl, methoximinoethyl and trifluoromethyl. A preferred aspect of such compounds are those of the formulae I.B14 and I.B15 (m=0, 1)

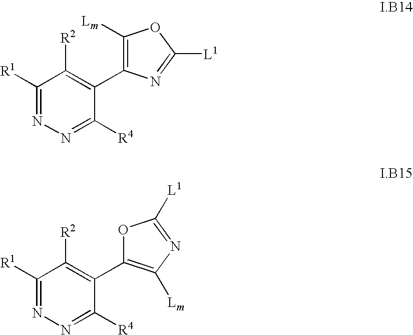

In one aspect of the compounds I.C, R³ is pyrazol-1-yl.

One aspect relates to compounds I.C in which R³ is pyrazol-1-yl which may be mono- to trisubstituted in the 3-, 4- or 5-position by identical or different L¹ and optionally by Lₘ, which here is preferably chlorine, bromine and/or methyl. Aspects of the compounds of the formula I relate to those of the formulae I.C1 and I.C2 (m=0, 1, 2).

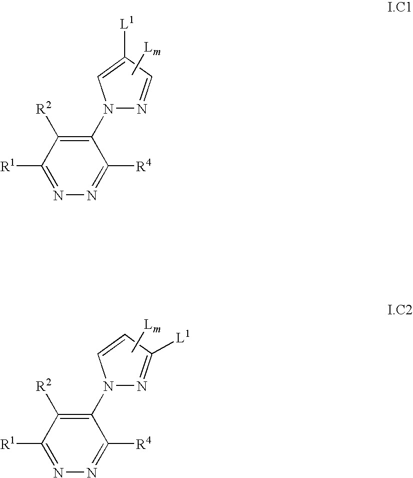

In a preferred aspect of the compounds I, in particular of the formulae I.A1 to I.B15, at least one group L is located ortho to the point of attachment of group R³ to the pyridazine skeleton, in particular chlorine, fluorine or methyl.

In a further preferred aspect, a heteroatom of the heteroaromatic R³ is located ortho to the point of attachment.

The index m is, if structurally possible, preferably 1 to 4, where the groups L may be identical of different. If the heteroaromatic groups R³ carry further substituents L in addition to a group L¹, these substituents L are preferably selected from the group consisting of: fluorine, chlorine, methyl, methoxy, cyano, nitro, alkoxycarbonyl, aminocarbonyl and haloalkyl. In a further aspect, the optional substituents L are selected from the group consisting of fluorine, chlorine, methyl and methoxy. In a further aspect, the optional substituents L are selected from the group consisting of chlorine, methyl and methoxy. A further aspect relates to heteroaromatic groups R³ which, in addition to a group L¹, are also substituted by chlorine.

R³ is in particular phenyl or pyridinyl, where these groups carry a substituent L¹ and 0, 1, 2, 3 or 4, preferably 0, 1 or 2, in particular 1 or 2, substituents L, where L¹ and L are defined as described above or below.

If R³ is phenyl or 2-pyridinyl, these rings carry the substituents L¹ preferably in the 3- or in particular the 4-position (based on the 1-position of the bond to the pyridazine ring; i.e. L¹ is particularly preferably located in the meta- or in particular para-position to this point of attachment). The phenyl or the 2-pyridinyl ring has optionally also 1 or 2 further substituents L. These are preferably attached in the 2- and/or 6-position of the phenyl ring (based on the 1-position of the bond to the pyridazine ring), i.e. located ortho to the point of attachment to the pyridazine ring, and in the case of the 2-pyridine ring preferably in the 6-position (based on the 1-position of the bond to the pyridazine ring).

In a preferred embodiment of the invention, the substituent L¹ of group R³ is a group L¹¹ of the formula

—Y^{α1}A^{α}—Y^{α2}]ₐ-A^{α}-T^{α}

where

A^{α} is C₁-C₄-alkylene;
Y^{α2} independently of one another are O, S or NR^{hα};

### T^{α} is OR^{hα}, SR^{hα} or NR^{hα}R^{jα};

each R^{hα} and R^{jα} is independently hydrogen or C₁-C₄-alkyl; and
a is 1, 2, 3 or 4.

C₁-C₄-alkylene in A^{α} is preferably methylene, 1,2-ethylene, 1,2- or 1,3-propylene or 1,4-n-butylene.

A^{α} is preferably methylene, 1,2-ethylene, 1,2-propylene or 1,3-propylene and in particular methylene or 1,2-ethylene.

Y^{α1} and Y^{α2} independently of one another are preferably O or NR^{hα}, If Y^{α1} is O, Y^{α2} is also preferably O. Moreover, in this case T^{α} is preferably OR^{hα}. If Y^{α1} is NR^{hα}R^{hα} and Y^{α2} is simultaneously O, T^{α} is in this case preferably OR^{hα}.

T^{α} is preferably OR^{hα} or NR^{hα}R^{jα}.

R^{hα} and R^{jα} independently of one another are preferably H, methyl or ethyl.

a is preferably 1, 2 or 3.

In another preferred embodiment of the invention, the substituent L¹ of group R³ is a group L¹² of the formula

Y^{β}-A^{β}-T^{β}

where

### Y^{β} is CH₂, O, S or NR^{hβ}R^{β};

A^{β} is C₁-C₈-alkylene;

### T^{β} is OR^{hβ}, NR^{hβ}R^{jβ} or OC(═O)-T^{3β};

### T^{3β} is R^{hβ}, OR^{hβ} or NR^{hβ}R^{jβ}; and

each R^{hβ} and R^{jβ} is independently H or C₁-C₄-alkyl.

Y^{β} is preferably CH₂ or O and especially O.

A^{β} is preferably C₁-C₆-alkylene, in particular C₁-C₄-alkylene.

T^{β} is preferably OR^{hβ} or NR^{hβ}R^{jβ}.

R^{hβ} and R^{jβ} independently of one another are preferably hydrogen, methyl or ethyl.

In another preferred embodiment of the invention, the substituent L¹ of group R³ is a group L¹³ of the formula

—Y^{1γ}-A^{γ}-T^{γ}

where

Y^{1γ} is —CONR^{hγ} or —COO;

A^{γ} is C₂-C₆-alkylene;

T^{γ} is OR^{hγ}, NR^{hγ}R^{jγ} or OC(═O)-T^{3γ};

T^{3γ} is R^{hγ}, OR^{hγ} or NR^{hγ}R^{jγ}; and

each R^{hγ} and R^{jγ} is independently H or C₁-C₄-alkyl.

In another preferred embodiment of the invention, the substituent L¹ of group R³ is a group L¹⁴ of the formula

C(Rⁱ)═C(Rⁱⁱ)—Y^{δ1}A^{δ}-Y^{δ2}]ₐ-A^{δ}-T^{δ}

where
Rⁱ, Rⁱⁱ is hydrogen;
A^{δ} is C₁-C₄-alkylene;
Y^{δ1}, Y^{δ2} independently of one another are O, S or NR^{hδ};

### T^{δ} is OR^{hδ}, SR^{hδ} or NR^{hδ}R^{jδ};

each R^{hδ} and R^{jδ} is independently hydrogen or C₁-C₄-alkyl; and
a is 1, 2, 3 or 4.
C₁-C₄-Alkylene in A^{δ} is preferably methylene, 1,2-ethylene, 1,2- or 1,3-propylene or 1,4-n-butylene.
A^{δ} is preferably methylene, 1,2-ethylene, 1,2-propylene or 1,3-propylene and in particular methylene or 1,2-ethylene.
Y^{δ1} and Y^{δ2} independently of one another are preferably O or NR^{hδ}. If Y^{δ1} is O, Y^{δ2} is also preferably O. Moreover, in this case T^{δ} is preferably OR^{hδ}. If Y^{δ1} is NR^{hδ}R^{jδ} and Y^{δ2} is simultaneously O, T^{δ} is in this case preferably OR^{hδ}.
T^{δ} is preferably OR^{hδ} or NR^{hδ}R^{jδ}.
R^{hδ} and R^{jδ} independently of one another are preferably H, methyl or ethyl.
a is preferably 1, 2 or 3.

In another preferred embodiment of the invention, the substituent L¹ of group R³ is a group L¹⁵ of the formula

C≡C—Y^{ε1}A^{ε}-Y^{ε2}]ₐ-A^{ε}-T^{ε}

where
A^{ε} is C₁-C₄-alkylene;
A^{ε1}, Y^{ε2} independently of one another are O, S or NR^{hε};

### T^{ε} is OR^{hε}, SR^{hε} or NR^{hε}R^{iε};

each R^{hε} and R^{iε} is independently hydrogen or C₁-C₄-alkyl; and
a is 1, 2, 3 or 4.
C₁-C₄-Alkylene in A^{δ} is preferably methylene, 1,2-ethylene, 1,2- or 1,3-propylene or 1,4-n-butylene.
A^{ε} is preferably methylene, 1,2-ethylene, 1,2-propylene or 1,3-propylene and in particular methylene or 1,2-ethylene.
Y^{ε1} and Y^{ε2} independently of one another are preferably O or NR^{hε}. If Y^{ε1} is O, Y^{ε2} is also preferably O. Moreover, in this case T^{ε} is preferably OR^{hε}. If Y^{ε1} is NR^{hε}R^{iε} and Y^{ε2} is simultaneously O, T^{ε} is in this case preferably OR^{hε}.
T^{ε} is preferably OR^{hε} or NR^{hε}R^{iε}.
R^{hε} and R^{iε} independently of one another are preferably H, methyl or ethyl.
a is preferably 1, 2 or 3.

The substituent L¹ of group R³ is particularly preferably a group L¹¹ or L¹².

Lₘ is preferably in each case independently selected from the preferred meanings given herein for Lₘ, where it may furthermore be preferred for L to be selected from the group consisting of halogen, nitro, cyano, C₁-C₄-alkyl, C₁-C₄-haloalkyl, in particular C₁-C₂-fluoroalkyl, C₁-C₄-alkoxy and C₁-C₄-alkoxycarbonyl, more preferably selected from the group consisting of nitro, cyano, fluorine, chlorine, bromine, methyl, ethyl, isopropyl, trifluoromethyl, fluoromethyl, methoxy and methoxycarbonyl. Preferred L in the ortho position are fluorine, chlorine, bromine, C₁-C₄-alkyl, in particular methyl or ethyl, C₁-C₂-fluoroalkyl, such as trifluoromethyl, and C₁-C₄-alkoxy, in particular methoxy or ethoxy.

Furthermore preferably, the substituents L which are attached to R³=heteroaryl, such as illustrated below, or R³=phenyl, are independently of one another:
halogen, cyano, nitro, amino, hydroxyl, formyl, carboxy, carbamoyl, thiocarbamoyl;
in each case straight-chain or branched alkyl, alkoxy, alkylthio, alkylsulfinyl or alkylsulfonyl having in each case 1 to 6 carbon atoms;
in each case straight-chain or branched alkenyl or alkenyloxy having in each case 2 to 6 carbon atoms;
in each case straight-chain or branched haloalkyl, haloalkoxy, haloalkylthio, haloalkylsulfinyl or haloalkylsulfonyl having in each case 1 to 6 carbon atoms and 1 to 13 identical or different halogen atoms;
in each case straight-chain or branched haloalkenyl or haloalkenyloxy having in each case 2 to 6 carbon atoms and 1 to 13 identical or different halogen atoms;
in each case straight-chain or branched haloalkenyl or haloalkenyloxy having in each case 2 to 6 carbon atoms and 1 to 11 identical or different halogen atoms;
in each case straight-chain or branched alkylamino, dialkylamino, alkylcarbonyl, alkylcarbonyloxy, alkoxycarbonyl, alkylsulfonyloxy, hydroximinoalkyl or alkoximinoalkyl having in each case 1 to 6 carbon atoms in the individual alkyl moieties;
cycloalkyl having 3 to 6 carbon atoms,
2,3-attached 1,3-propanediyl, 1,4-butanediyl, methylenedioxy
(—O—CH₂—O—) or 1,2-ethylenedioxy (—O—CH₂—CH₂—O—), where these groups may be mono- or polysubstituted by identical or different substituents from the group consisting of halogen, alkyl having 1 to 4 carbon atoms and/or haloalkyl having 1 to 4 carbon atoms and 1 to 9 identical or different halogen atoms.

Suitable Lₘ are in particular the following groups: halogen, such as fluorine or chlorine; cyano; nitro; alkoxycarbonyl; C₁-C₄-alkyl, such as methyl; C₁-C₄-haloalkyl, such as trifluoromethyl; C₁-C₄-alkoxy, such as methoxy.

Examples of preferred compounds the general formula I are those of the formulae I.a and I.b

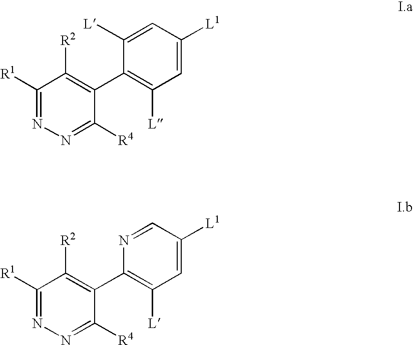

where the variables R¹, R² and R⁴ and L¹ have the general or preferred meanings given above and L′ and L″ are hydrogen or have one of the general or preferred meanings given for L.

In particular with a view to their use, preference is given to the compounds I compiled in the tables below. Moreover, the groups mentioned for a substituent in the tables are per se, independently of the combination in which they are mentioned, a particularly preferred aspect of the substituent in question.

### Table 1

Compounds of the formula I.a in which R¹ and R⁴ are Cl, L′ and L″ are H, L¹ is —(OCH₂)₂—OH and R² for a compound corresponds in each case to one row of Table A

### Table 2

Compounds of the formula I.a in which R¹ and R⁴ are Cl, L′ and L″ are H, L¹ is —(OCH₂)₂—OCH₃ and R² for a compound corresponds in each case to one row of Table A

### Table 3

Compounds of the formula I.a in which R¹ and R⁴ are Cl, L′ and L″ are H, L¹ is —(OCH₂)₂—OC₂H₅ and R² for a compound corresponds in each case to one row of Table A

### Table 4

Compounds of the formula I.a in which R¹ and R⁴ are Cl, L′ and L″ are H, L¹ is —(OCH₂)₃—OH and R² for a compound corresponds in each case to one row of Table A

### Table 5

Compounds of the formula I.a in which R¹ and R⁴ are Cl, L′ and L″ are H, L¹ is —(OCH₂)₃—OCH₃ and R² for a compound corresponds in each case to one row of Table A

### Table 6

Compounds of the formula I.a in which R¹ and R⁴ are Cl, L′ and L″ are H, L¹ is —(OCH₂)₃—OC₂H₅ and R² for a compound corresponds in each case to one row of Table A

### Table 7

Compounds of the formula I.a in which R¹ and R⁴ are Cl, L′ and L″ are H, L¹ is —O—CH₂CH₂—OH and R² for a compound corresponds in each case to one row of Table A

### Table 8

Compounds of the formula I.a in which R¹ and R⁴ are Cl, L′ and L″ are H, L¹ is —O—CH₂CH₂—OCH₃ and R² for a compound corresponds in each case to one row of Table A

### Table 9

Compounds of the formula I.a in which R¹ and R⁴ are Cl, L′ and L″ are H, L¹ is —O—CH₂CH₂—OC₂H₅ and R² for a compound corresponds in each case to one row of Table A

### Table 10

Compounds of the formula I.a in which R¹ and R⁴ are Cl, L′ and L″ are H, L¹ is —(O—CH₂CH₂)₂—OH and R² for a compound corresponds in each case to one row of Table A

### Table 11

Compounds of the formula I.a in which R¹ and R⁴ are Cl, L′ and L″ are H, L¹ is —(O—CH₂CH₂)₂—OCH₃ and R² for a compound corresponds in each case to one row of Table A

### Table 12

Compounds of the formula I.a in which R¹ and R⁴ are Cl, L′ and L″ are H, L¹ is —(O—CH₂CH₂)₂—OC₂H₅ and R² for a compound corresponds in each case to one row of Table A

### Table 13

Compounds of the formula I.a in which R¹ and R⁴ are Cl, L′ and L″ are H, L¹ is —O—CH₂CH₂—NHCH₃ and R² for a compound corresponds in each case to one row of Table A

### Table 14

Compounds of the formula I.a in which R¹ and R⁴ are Cl, L′ and L″ are H, L¹ is —O—CH₂CH₂—N(CH₃)₂ and R² for a compound corresponds in each case to one row of Table A

### Table 15

Compounds of the formula I.a in which R¹ and R⁴ are Cl, L′ and L″ are H, L¹ is —O—CH₂CH₂—NHC₂H₅ and R² for a compound corresponds in each case to one row of Table A

### Table 16

Compounds of the formula I.a in which R¹ and R⁴ are Cl, L′ and L″ are H, L¹ is —O—CH₂CH₂—N(C₂H₅)₂ and R² for a compound corresponds in each case to one row of Table A

### Table 17

Compounds of the formula I.a in which R¹ and R⁴ are Cl, L′ and L″ are H, L¹ is —O—CH₂CH₂CH₂—NHCH₃ and R² for a compound corresponds in each case to one row of Table A

### Table 18

Compounds of the formula I.a in which R¹ and R⁴ are Cl, L′ and L″ are H, L¹ is —O—CH₂CH₂CH₂—N(CH₃)₂ and R² for a compound corresponds in each case to one row of Table A

### Table 19

Compounds of the formula I.a in which R¹ and R⁴ are Cl, L′ and L″ are H, L¹ is —O—CH₂CH₂CH₂—NHC₂H₅ and R² for a compound corresponds in each case to one row of Table A

### Table 20

Compounds of the formula I.a in which R¹ and R⁴ are Cl, L′ and L″ are H, L¹ is —O—CH₂CH₂CH₂—N(C₂H₅)₂ and R² for a compound corresponds in each case to one row of Table A

### Table 21

Compounds of the formula I.a in which R¹ and R⁴ are Cl, L′ and L″ are H, L¹ is —O—CH₂CH₂CH₂CH₂—NHCH₃ and R² for a compound corresponds in each case to one row of Table A

### Table 22

Compounds of the formula I.a in which R¹ and R⁴ are Cl, L′ and L″ are H, L¹ is —O—CH₂CH₂CH₂CH₂—N(CH₃)₂ and R² for a compound corresponds in each case to one row of Table A

### Table 23

Compounds of the formula I.a in which R¹ and R⁴ are Cl, L′ and L″ are H, L¹ is —O—CH₂CH₂CH₂CH₂—NHC₂H₅ and R² for a compound corresponds in each case to one row of Table A

### Table 24

Compounds of the formula I.a in which R¹ and R⁴ are Cl, L′ and L″ are H, L¹ is —O—CH₂CH₂CH₂CH₂—N(C₂H₅)₂ and R² for a compound corresponds in each case to one row of Table A

### Table 25

Compounds of the formula I.a in which R¹, R⁴ and L′ are Cl, L″ is H, L¹ is —(OCH₂)₂—OH and R² for a compound corresponds in each case to one row of Table A

### Table 26

Compounds of the formula I.a in which R¹, R⁴ and L′ are Cl, L″ is H, L¹ is —(OCH₂)₂—OCH₃ and R² for a compound corresponds in each case to one row of Table A

### Table 27

Compounds of the formula I.a in which R¹, R⁴ and L′ are Cl, L″ is H, L¹ is —(OCH₂)₂—OC₂H₅ and R² for a compound corresponds in each case to one row of Table A

### Table 28

Compounds of the formula I.a in which R¹, R⁴ and L′ are Cl, L″ is H, L¹ is —(OCH₂)₃—OH and R² for a compound corresponds in each case to one row of Table A

### Table 29

Compounds of the formula I.a in which R¹, R⁴ and L′ are Cl, L″ is H, L¹ is —(OCH₂)₃—OCH₃ and R² for a compound corresponds in each case to one row of Table A

### Table 30

Compounds of the formula I.a in which R¹, R⁴ and L′ are Cl, L″ is H, L¹ is —(OCH₂)₃—OC₂H₅ and R² for a compound corresponds in each case to one row of Table A

### Table 31

Compounds of the formula I.a in which R¹, R⁴ and L′ are Cl, L″ is H, L¹ is —O—CH₂CH₂—OH and R² for a compound corresponds in each case to one row of Table A

### Table 32

Compounds of the formula I.a in which R¹, R⁴ and L′ are Cl, L″ is H, L¹ is —O—CH₂CH₂—OCH₃ and R² for a compound corresponds in each case to one row of Table A

### Table 33

Compounds of the formula I.a in which R¹, R⁴ and L′ are Cl, L″ is H, L¹ is —O—CH₂CH₂—OC₂H₅ and R² for a compound corresponds in each case to one row of Table A

### Table 34

Compounds of the formula I.a in which R¹, R⁴ and L′ are Cl, L″ is H, L¹ is —(O—CH₂CH₂)₂—OH and R² for a compound corresponds in each case to one row of Table A

### Table 35

Compounds of the formula I.a in which R¹, R⁴ and L′ are Cl, L″ is H, L¹ is —(O—CH₂CH₂)₂—OCH₃ and R² for a compound corresponds in each case to one row of Table A

### Table 36

Compounds of the formula I.a in which R¹, R⁴ and L′ are Cl, L″ is H, L¹ is —(O—CH₂CH₂)₂—OC₂H₅ and R² for a compound corresponds in each case to one row of Table A

### Table 37

Compounds of the formula I.a in which R¹, R⁴ and L′ are Cl, L″ is H, L¹ is —O—CH₂CH₂—NHCH₃ and R² for a compound corresponds in each case to one row of Table A

### Table 38

Compounds of the formula I.a in which R¹, R⁴ and L′ are Cl, L″ is H, L¹ is —O—CH₂CH₂—N(CH₃)₂ and R² for a compound corresponds in each case to one row of Table A

### Table 39

Compounds of the formula I.a in which R¹, R⁴ and L′ are Cl, L″ is H, L¹ is —O—CH₂CH₂—NHC₂H₅ and R² for a compound corresponds in each case to one row of Table A

### Table 40

Compounds of the formula I.a in which R¹, R⁴ and L′ are Cl, L″ is H, L¹ is —O—CH₂CH₂—N(C₂H₅)₂ and R² for a compound corresponds in each case to one row of Table A

### Table 41

Compounds of the formula I.a in which R¹, R⁴ and L′ are Cl, L″ is H, L¹ is —O—CH₂CH₂CH₂—NHCH₃ and R² for a compound corresponds in each case to one row of Table A

### Table 42

Compounds of the formula I.a in which R¹, R⁴ and L′ are Cl, L″ is H, L¹ is —O—CH₂CH₂CH₂—N(CH₃)₂ and R² for a compound corresponds in each case to one row of Table A

### Table 43

Compounds of the formula I.a in which R¹, R⁴ and L′ are Cl, L″ is H, L¹ is —O—CH₂CH₂CH₂—NHC₂H₅ and R² for a compound corresponds in each case to one row of Table A

### Table 44

Compounds of the formula I.a in which R¹, R⁴ and L′ are Cl, L″ is H, L¹ is —O—CH₂CH₂CH₂—N(C₂H₅)₂ and R² for a compound corresponds in each case to one row of Table A

### Table 45

Compounds of the formula I.a in which R¹, R⁴ and L′ are Cl, L″ is H, L¹ is —O—CH₂CH₂CH₂CH₂—NHCH₃ and R² for a compound corresponds in each case to one row of Table A

### Table 46

Compounds of the formula I.a in which R¹, R⁴ and L′ are Cl, L″ is H, L¹ is —O—CH₂CH₂CH₂CH₂—N(CH₃)₂ and R² for a compound corresponds in each case to one row of Table A

### Table 47

Compounds of the formula I.a in which R¹, R⁴ and L′ are Cl, L″ is H, L¹ is —O—CH₂CH₂CH₂CH₂—NHC₂H₅ and R² for a compound corresponds in each case to one row of Table A

### Table 48

Compounds of the formula I.a in which R¹, R⁴ and L′ are Cl, L″ is H, L¹ is —O—CH₂CH₂CH₂CH₂—N(C₂H₅)₂ and R² for a compound corresponds in each case to one row of Table A

### Table 49

Compounds of the formula I.a in which R¹ and R⁴ are Cl, L′ is F, L″ is H, L¹ is —(OCH₂)₂—OH and R² for a compound corresponds in each case to one row of Table A

### Table 50

Compounds of the formula I.a in which R¹ and R⁴ are Cl, L′ is F, L″ is H, L¹ is —(OCH₂)₂—OCH₃ and R² for a compound corresponds in each case to one row of Table A

### Table 51

Compounds of the formula I.a in which R¹ and R⁴ are Cl, L′ is F, L″ is H, L¹ is —(OCH₂)₂—OC₂H₅ and R² for a compound corresponds in each case to one row of Table A

### Table 52

Compounds of the formula I.a in which R¹ and R⁴ are Cl, L′ is F, L″ is H, L¹ is —(OCH₂)₃—OH and R² for a compound corresponds in each case to one row of Table A

### Table 53

Compounds of the formula I.a in which R¹ and R⁴ are Cl, L′ is F, L″ is H, L¹ is —(OCH₂)₃—OCH₃ and R² for a compound corresponds in each case to one row of Table A

### Table 54

Compounds of the formula I.a in which R¹ and R⁴ are Cl, L′ is F, L″ is H, L¹ is —(OCH₂)₃—OC₂H₅ and R² for a compound corresponds in each case to one row of Table A

### Table 55

Compounds of the formula I.a in which R¹ and R⁴ are Cl, L′ is F, L″ is H, L¹ is —O—CH₂CH₂—OH and R² for a compound corresponds in each case to one row of Table A

### Table 56

Compounds of the formula I.a in which R¹ and R⁴ are Cl, L′ is F, L″ is H, L¹ is —O—CH₂CH₂—OCH₃ and R² for a compound corresponds in each case to one row of Table A

### Table 57

Compounds of the formula I.a in which R¹ and R⁴ are Cl, L′ is F, L″ is H, L¹ is —O—CH₂CH₂—OC₂H₅ and R² for a compound corresponds in each case to one row of Table A

### Table 58

Compounds of the formula I.a in which R¹ and R⁴ are Cl, L′ is F, L″ is H, L¹ is —(O—CH₂CH₂)₂—OH and R² for a compound corresponds in each case to one row of Table A

### Table 59

Compounds of the formula I.a in which R¹ and R⁴ are Cl, L′ is F, L″ is H, L¹ is —(O—CH₂CH₂)₂—OCH₃ and R² for a compound corresponds in each case to one row of Table A

### Table 60

Compounds of the formula I.a in which R¹ and R⁴ are Cl, L′ is F, L″ is H, L¹ is —(O—CH₂CH₂)₂—OC₂H₅ and R² for a compound corresponds in each case to one row of Table A

### Table 61

Compounds of the formula I.a in which R¹ and R⁴ are Cl, L′ is F, L″ is H, L¹ is —O—CH₂CH₂—NHCH₃ and R² for a compound corresponds in each case to one row of Table A

### Table 62

Compounds of the formula I.a in which R¹ and R⁴ are Cl, L′ is F, L″ is H, L¹ is —O—CH₂CH₂—N(CH₃)₂ and R² for a compound corresponds in each case to one row of Table A

### Table 63

Compounds of the formula I.a in which R¹ and R⁴ are Cl, L′ is F, L″ is H, L¹ is —O—CH₂CH₂—NHC₂H₅ and R² for a compound corresponds in each case to one row of Table A

### Table 64

Compounds of the formula I.a in which R¹ and R⁴ are Cl, L′ is F, L″ is H, L¹ is —O—CH₂CH₂—N(C₂H₅)₂ and R² for a compound corresponds in each case to one row of Table A

### Table 65

Compounds of the formula I.a in which R¹ and R⁴ are Cl, L′ is F, L″ is H, L¹ is —O—CH₂CH₂CH₂—NHCH₃ and R² for a compound corresponds in each case to one row of Table A

### Table 66

Compounds of the formula I.a in which R¹ and R⁴ are Cl, L′ is F, L″ is H, L¹ is —O—CH₂CH₂CH₂—N(CH₃)₂ and R² for a compound corresponds in each case to one row of Table A

### Table 67

Compounds of the formula I.a in which R¹ and R⁴ are Cl, L′ is F, L″ is H, L¹ is —O—CH₂CH₂CH₂—NHC₂H₅ and R² for a compound corresponds in each case to one row of Table A

### Table 68

Compounds of the formula I.a in which R¹ and R⁴ are Cl, L′ is F, L″ is H, L¹ is —O—CH₂CH₂CH₂—N(C₂H₅)₂ and R² for a compound corresponds in each case to one row of Table A

### Table 69

Compounds of the formula I.a in which R¹ and R⁴ are Cl, L′ is F, L″ is H, L¹ is —O—CH₂CH₂CH₂CH₂—NHCH₃ and R² for a compound corresponds in each case to one row of Table A

### Table 70

Compounds of the formula I.a in which R¹ and R⁴ are Cl, L′ is F, L″ is H, L¹ is —O—CH₂CH₂CH₂CH₂—N(CH₃)₂ and R² for a compound corresponds in each case to one row of Table A

### Table 71

Compounds of the formula I.a in which R¹ and R⁴ are Cl, L′ is F, L″ is H, L¹ is —O—CH₂CH₂CH₂CH₂—NHC₂H₅ and R² for a compound corresponds in each case to one row of Table A

### Table 72

Compounds of the formula I.a in which R¹ and R⁴ are Cl, L′ is F, L″ is H, L¹ is —O—CH₂CH₂CH₂CH₂—N(C₂H₅)₂ and R² for a compound corresponds in each case to one row of Table A

### Table 73

Compounds of the formula I.a in which R¹ and R⁴ are Cl, L′ is CH₃, L″ is H, L¹ is —(OCH₂)₂—OH and R² for a compound corresponds in each case to one row of Table A

### Table 74

Compounds of the formula I.a in which R¹ and R⁴ are Cl, L′ is CH₃, L″ is H, L¹ is —(OCH₂)₂—OCH₃ and R² for a compound corresponds in each case to one row of Table A

### Table 75

Compounds of the formula I.a in which R¹ and R⁴ are Cl, L′ is CH₃, L″ is H, L¹ is —(OCH₂)₂—OC₂H₅ and R² for a compound corresponds in each case to one row of Table A

### Table 76

Compounds of the formula I.a in which R¹ and R⁴ are Cl, L′ is CH₃, L″ is H, L¹ is —(OCH₂)₃—OH and R² for a compound corresponds in each case to one row of Table A

### Table 77

Compounds of the formula I.a in which R¹ and R⁴ are Cl, L′ is CH₃, L″ is H, L¹ is —(OCH₂)₃—OCH₃ and R² for a compound corresponds in each case to one row of Table A

### Table 78

Compounds of the formula I.a in which R¹ and R⁴ are Cl, L′ is CH₃, L″ is H, L¹ is —(OCH₂)₃—OC₂H₅ and R² for a compound corresponds in each case to one row of Table A

### Table 79

Compounds of the formula I.a in which R¹ and R⁴ are Cl, L′ is CH₃, L″ is H, L¹ is —O—CH₂CH₂—OH and R² for a compound corresponds in each case to one row of Table A

### Table 80

Compounds of the formula I.a in which R¹ and R⁴ are Cl, L′ is CH₃, L″ is H, L¹ is —O—CH₂CH₂—OCH₃ and R² for a compound corresponds in each case to one row of Table A

### Table 81

Compounds of the formula I.a in which R¹ and R⁴ are Cl, L′ is CH₃, L″ is H, L¹ is —O—CH₂CH₂—OC₂H₅ and R² for a compound corresponds in each case to one row of Table A

### Table 82

Compounds of the formula I.a in which R¹ and R⁴ are Cl, L′ is CH₃, L″ is H, L¹ is —(O—CH₂CH₂)₂—OH and R² for a compound corresponds in each case to one row of Table A

### Table 83

Compounds of the formula I.a in which R¹ and R⁴ are Cl, L′ is CH₃, L″ is H, L¹ is —(O—CH₂CH₂)₂—OCH₃ and R² for a compound corresponds in each case to one row of Table A

### Table 84

Compounds of the formula I.a in which R¹ and R⁴ are Cl, L′ is CH₃, L″ is H, L¹ is —(O—CH₂CH₂)₂—OC₂H₅ and R² for a compound corresponds in each case to one row of Table A

### Table 85

Compounds of the formula I.a in which R¹ and R⁴ are Cl, L′ is CH₃, L″ is H, L¹ is —O—CH₂CH₂—NHCH₃ and R² for a compound corresponds in each case to one row of Table A

### Table 86

Compounds of the formula I.a in which R¹ and R⁴ are Cl, L′ is CH₃, L″ is H, L¹ is —O—CH₂CH₂—N(CH₃)₂ and R² for a compound corresponds in each case to one row of Table A

### Table 87

Compounds of the formula I.a in which R¹ and R⁴ are Cl, L′ is CH₃, L″ is H, L¹ is —O—CH₂CH₂—NHC₂H₅ and R² for a compound corresponds in each case to one row of Table A

### Table 88

Compounds of the formula I.a in which R¹ and R⁴ are Cl, L′ is CH₃, L″ is H, L¹ is —O—CH₂CH₂—N(C₂H₅)₂ and R² for a compound corresponds in each case to one row of Table A

### Table 89

Compounds of the formula I.a in which R¹ and R⁴ are Cl, L′ is CH₃, L″ is H, L¹ is —O—CH₂CH₂CH₂—NHCH₃ and R² for a compound corresponds in each case to one row of Table A

### Table 90

Compounds of the formula I.a in which R¹ and R⁴ are Cl, L′ is CH₃, L″ is H, L¹ is —O—CH₂CH₂CH₂—N(CH₃)₂ and R² for a compound corresponds in each case to one row of Table A

### Table 91

Compounds of the formula I.a in which R¹ and R⁴ are Cl, L′ is CH₃, L″ is H, L¹ is —O—CH₂CH₂CH₂—NHC₂H₅ and R² for a compound corresponds in each case to one row of Table A

### Table 92

Compounds of the formula I.a in which R¹ and R⁴ are Cl, L′ is CH₃, L″ is H, L¹ is —O—CH₂CH₂CH₂—N(C₂H₅)₂ and R² for a compound corresponds in each case to one row of Table A

### Table 93

Compounds of the formula I.a in which R¹ and R⁴ are Cl, L′ is CH₃, L″ is H, L¹ is —O—CH₂CH₂CH₂CH₂—NHCH₃ and R² for a compound corresponds in each case to one row of Table A

### Table 94

Compounds of the formula I.a in which R¹ and R⁴ are Cl, L′ is CH₃, L″ is H, L¹ is —O—CH₂CH₂CH₂CH₂—N(CH₃)₂ and R² for a compound corresponds in each case to one row of Table A

### Table 95

Compounds of the formula I.a in which R¹ and R⁴ are Cl, L′ is CH₃, L″ is H, L¹ is —O—CH₂CH₂CH₂CH₂—NHC₂H₅ and R² for a compound corresponds in each case to one row of Table A

### Table 96

Compounds of the formula I.a in which R¹ and R⁴ are Cl, L′ is CH₃, L″ is H, L¹ is —O—CH₂CH₂CH₂CH₂—N(C₂H₅)₂ and R² for a compound corresponds in each case to one row of Table A

### Table 97

Compounds of the formula I.a in which R¹ and R⁴ are Cl, L′ and L″ are F, L¹ is —(OCH₂)₂—OH and R² for a compound corresponds in each case to one row of Table A

### Table 98

Compounds of the formula I.a in which R¹ and R⁴ are Cl, L′ and L″ are F, L¹ is —(OCH₂)₂—OCH₃ and R² for a compound corresponds in each case to one row of Table A

### Table 99

Compounds of the formula I.a in which R¹ and R⁴ are Cl, L′ and L″ are F, L¹ is —(OCH₂)₂—OC₂H₅ and R² for a compound corresponds in each case to one row of Table A

### Table 100

Compounds of the formula I.a in which R¹ and R⁴ are Cl, L′ and L″ are F, L¹ is —(OCH₂)₃—OH and R² for a compound corresponds in each case to one row of Table A

### Table 101

Compounds of the formula I.a in which R¹ and R⁴ are Cl, L′ and L″ are F, L¹ is —(OCH₂)₃—OCH₃ and R² for a compound corresponds in each case to one row of Table A

### Table 102

Compounds of the formula I.a in which R¹ and R⁴ are Cl, L′ and L″ are F, L¹ is —(OCH₂)₃—OC₂H₅ and R² for a compound corresponds in each case to one row of Table A

### Table 103

Compounds of the formula I.a in which R¹ and R⁴ are Cl, L′ and L″ are F, L¹ is —O—CH₂CH₂—OH and R² for a compound corresponds in each case to one row of Table A

### Table 104

Compounds of the formula I.a in which R¹ and R⁴ are Cl, L′ and L″ are F, L¹ is —O—CH₂CH₂—OCH₃ and R² for a compound corresponds in each case to one row of Table A

### Table 105

Compounds of the formula I.a in which R¹ and R⁴ are Cl, L′ and L″ are F, L¹ is —O—CH₂CH₂—OC₂H₅ and R² for a compound corresponds in each case to one row of Table A

### Table 106

Compounds of the formula I.a in which R¹ and R⁴ are Cl, L′ and L″ are F, L¹ is —(O—CH₂CH₂)₂—OH and R² for a compound corresponds in each case to one row of Table A

### Table 107

Compounds of the formula I.a in which R¹ and R⁴ are Cl, L′ and L″ are F, L¹ is —(O—CH₂CH₂)₂—OCH₃ and R² for a compound corresponds in each case to one row of Table A

### Table 108

Compounds of the formula I.a in which R¹ and R⁴ are Cl, L′ and L″ are F, L¹ is —(O—CH₂CH₂)₂—OC₂H₅ and R² for a compound corresponds in each case to one row of Table A

### Table 109

Compounds of the formula I.a in which R¹ and R⁴ are Cl, L′ and L″ are F, L¹ is —O—CH₂CH₂—NHCH₃ and R² for a compound corresponds in each case to one row of Table A

### Table 110

Compounds of the formula I.a in which R¹ and R⁴ are Cl, L′ and L″ are F, L¹ is —O—CH₂CH₂—N(CH₃)₂ and R² for a compound corresponds in each case to one row of Table A

### Table 111

Compounds of the formula I.a in which R¹ and R⁴ are Cl, L′ and L″ are F, L¹ is —O—CH₂CH₂—NHC₂H₅ and R² for a compound corresponds in each case to one row of Table A

### Table 112

Compounds of the formula I.a in which R¹ and R⁴ are Cl, L′ and L″ are F, L¹ is —O—CH₂CH₂—N(C₂H₅)₂ and R² for a compound corresponds in each case to one row of Table A

### Table 113

Compounds of the formula I.a in which R¹ and R⁴ are Cl, L′ and L″ are F, L¹ is —O—CH₂CH₂CH₂—NHCH₃ and R² for a compound corresponds in each case to one row of Table A

### Table 114

Compounds of the formula I.a in which R¹ and R⁴ are Cl, L′ and L″ are F, L¹ is —O—CH₂CH₂CH₂—N(CH₃)₂ and R² for a compound corresponds in each case to one row of Table A

### Table 115

Compounds of the formula I.a in which R¹ and R⁴ are Cl, L′ and L″ are F, L¹ is —O—CH₂CH₂CH₂—NHC₂H₅ and R² for a compound corresponds in each case to one row of Table A

### Table 116

Compounds of the formula I.a in which R¹ and R⁴ are Cl, L′ and L″ are F, L¹ is —O—CH₂CH₂CH₂—N(C₂H₅)₂ and R² for a compound corresponds in each case to one row of Table A

### Table 117

Compounds of the formula I.a in which R¹ and R⁴ are Cl, L′ and L″ are F, L¹ is —O—CH₂CH₂CH₂CH₂—NHCH₃ and R² for a compound corresponds in each case to one row of Table A

### Table 118

Compounds of the formula I.a in which R¹ and R⁴ are Cl, L′ and L″ are F, L¹ is —O—CH₂CH₂CH₂CH₂—N(CH₃)₂ and R² for a compound corresponds in each case to one row of Table A

### Table 119

Compounds of the formula I.a in which R¹ and R⁴ are Cl, L′ and L″ are F, L¹ is —O—CH₂CH₂CH₂CH₂—NHC₂H₅ and R² for a compound corresponds in each case to one row of Table A

### Table 120

Compounds of the formula I.a in which R¹ and R⁴ are Cl, L′ and L″ are F, L¹ is —O—CH₂CH₂CH₂CH₂—N(C₂H₅)₂ and R² for a compound corresponds in each case to one row of Table A

### Table 121

Compounds of the formula I.a in which R¹, R⁴ and L′ are Cl and L″ is F, L¹ is —(OCH₂)₂—OH and R² for a compound corresponds in each case to one row of Table A

### Table 122

Compounds of the formula I.a in which R¹, R⁴ and L′ are Cl and L″ is F, L¹ is —(OCH₂)₂—OCH₃ and R² for a compound corresponds in each case to one row of Table A

### Table 123

Compounds of the formula I.a in which R¹, R⁴ and L′ are Cl and L″ is F, L¹ is —(OCH₂)₂—OC₂H₅ and R² for a compound corresponds in each case to one row of Table A

### Table 124

Compounds of the formula I.a in which R¹, R⁴ and L′ are Cl and L″ is F, L¹ is —(OCH₂)₃—OH and R² for a compound corresponds in each case to one row of Table A

### Table 125

Compounds of the formula I.a in which R¹, R⁴ and L′ are Cl and L″ is F, L¹ is —(OCH₂)₃—OCH₃ and R² for a compound corresponds in each case to one row of Table A

### Table 126

Compounds of the formula I.a in which R¹, R⁴ and L′ are Cl and L″ is F, L¹ is —(OCH₂)₃—OC₂H₅ and R² for a compound corresponds in each case to one row of Table A

### Table 127

Compounds of the formula I.a in which R¹, R⁴ and L′ are Cl and L″ is F, L¹ is —O—CH₂CH₂—OH and R² for a compound corresponds in each case to one row of Table A

### Table 128

Compounds of the formula I.a in which R¹, R⁴ and L′ are Cl and L″ is F, L¹ is —O—CH₂CH₂—OCH₃ and R² for a compound corresponds in each case to one row of Table A

### Table 129

Compounds of the formula I.a in which R¹, R⁴ and L′ are Cl and L″ is F, L¹ is —O—CH₂CH₂—OC₂H₅ and R² for a compound corresponds in each case to one row of Table A

### Table 130

Compounds of the formula I.a in which R¹, R⁴ and L′ are Cl and L″ is F, L¹ is —(O—CH₂CH₂)₂—OH and R² for a compound corresponds in each case to one row of Table A

### Table 131

Compounds of the formula I.a in which R¹, R⁴ and L′ are Cl and L″ is F, L¹ is —(O—CH₂CH₂)₂—OCH₃ and R² for a compound corresponds in each case to one row of Table A

### Table 132

Compounds of the formula I.a in which R¹, R⁴ and L′ are Cl and L″ is F, L¹ is —(O—CH₂CH₂)₂—OC₂H₅ and R² for a compound corresponds in each case to one row of Table A

### Table 133

Compounds of the formula I.a in which R¹, R⁴ and L′ are Cl and L″ is F, L¹ is —O—CH₂CH₂—NHCH₃ and R² for a compound corresponds in each case to one row of Table A

### Table 134

Compounds of the formula I.a in which R¹, R⁴ and L′ are Cl and L″ is F, L¹ is —O—CH₂CH₂—N(CH₃)₂ and R² for a compound corresponds in each case to one row of Table A

### Table 135

Compounds of the formula I.a in which R¹, R⁴ and L′ are Cl and L″ is F, L¹ is —O—CH₂CH₂—NHC₂H₅ and R² for a compound corresponds in each case to one row of Table A

### Table 136

Compounds of the formula I.a in which R¹, R⁴ and L′ are Cl and L″ is F, L¹ is —O—CH₂CH₂—N(C₂H₅)₂ and R² for a compound corresponds in each case to one row of Table A

### Table 137

Compounds of the formula I.a in which R¹, R⁴ and L′ are Cl and L″ is F, L¹ is —O—CH₂CH₂CH₂—NHCH₃ and R² for a compound corresponds in each case to one row of Table A

### Table 138

Compounds of the formula I.a in which R¹, R⁴ and L′ are Cl and L″ is F, L¹ is —O—CH₂CH₂CH₂—N(CH₃)₂ and R² for a compound corresponds in each case to one row of Table A

### Table 139

Compounds of the formula I.a in which R¹, R⁴ and L′ are Cl and L″ is F, L¹ is —O—CH₂CH₂CH₂—NHC₂H₅ and R² for a compound corresponds in each case to one row of Table A

### Table 140

Compounds of the formula I.a in which R¹, R⁴ and L′ are Cl and L″ is F, L¹ is —O—CH₂CH₂CH₂—N(C₂H₅)₂ and R² for a compound corresponds in each case to one row of Table A

### Table 141

Compounds of the formula I.a in which R¹, R⁴ and L′ are Cl and L″ is F, L¹ is —O—CH₂CH₂CH₂CH₂—NHCH₃ and R² for a compound corresponds in each case to one row of Table A

### Table 142

Compounds of the formula I.a in which R¹, R⁴ and L′ are Cl and L″ is F, L¹ is —O—CH₂CH₂CH₂CH₂—N(CH₃)₂ and R² for a compound corresponds in each case to one row of Table A

### Table 143

Compounds of the formula I.a in which R¹, R⁴ and L′ are Cl and L″ is F, L¹ is —O—CH₂CH₂CH₂CH₂—NHC₂H₅ and R² for a compound corresponds in each case to one row of Table A

### Table 144

Compounds of the formula I.a in which R¹, R⁴ and L′ are Cl and L″ is F, L¹ is —O—CH₂CH₂CH₂CH₂—N(C₂H₅)₂ and R² for a compound corresponds in each case to one row of Table A

### Table 145

Compounds of the formula I.a in which R¹ and R⁴ are Cl, L′ is CH₃, L″ is F, L¹ is —(OCH₂)₂—OH and R² for a compound corresponds in each case to one row of Table A

### Table 146

Compounds of the formula I.a in which R¹ and R⁴ are Cl, L′ is CH₃, L″ is F, L¹ is —(OCH₂)₂—OCH₃ and R² for a compound corresponds in each case to one row of Table A

### Table 147

Compounds of the formula I.a in which R¹ and R⁴ are Cl, L′ is CH₃, L″ is F, L¹ is —(OCH₂)₂—OC₂H₅ and R² for a compound corresponds in each case to one row of Table A

### Table 148

Compounds of the formula I.a in which R¹ and R⁴ are Cl, L′ is CH₃, L″ is F, L¹ is —(OCH₂)₃—OH and R² for a compound corresponds in each case to one row of Table A

### Table 149

Compounds of the formula I.a in which R¹ and R⁴ are Cl, L′ is CH₃, L″ is F, L¹ is —(OCH₂)₃—OCH₃ and R² for a compound corresponds in each case to one row of Table A

### Table 150

Compounds of the formula I.a in which R¹ and R⁴ are Cl, L′ is CH₃, L″ is F, L¹ is —(OCH₂)₃—OC₂H₅ and R² for a compound corresponds in each case to one row of Table A

### Table 151

Compounds of the formula I.a in which R¹ and R⁴ are Cl, L′ is CH₃, L″ is F, L¹ is —O—CH₂CH₂—OH and R² for a compound corresponds in each case to one row of Table A

### Table 152

Compounds of the formula I.a in which R¹ and R⁴ are Cl, L′ is CH₃, L″ is F, L¹ is —O—CH₂CH₂—OCH₃ and R² for a compound corresponds in each case to one row of Table A

### Table 153

Compounds of the formula I.a in which R¹ and R⁴ are Cl, L′ is CH₃, L″ is F, L¹ is —O—CH₂CH₂—OC₂H₅ and R² for a compound corresponds in each case to one row of Table A

### Table 154

Compounds of the formula I.a in which R¹ and R⁴ are Cl, L′ is CH₃, L″ is F, L¹ is —(O—CH₂CH₂)₂—OH and R² for a compound corresponds in each case to one row of Table A

### Table 155

Compounds of the formula I.a in which R¹ and R⁴ are Cl, L′ is CH₃, L″ is F, L¹ is —(O—CH₂CH₂)₂—OCH₃ and R² for a compound corresponds in each case to one row of Table A

### Table 156

Compounds of the formula I.a in which R¹ and R⁴ are Cl, L′ is CH₃, L″ is F, L¹ is —(O—CH₂CH₂)₂—OC₂H₅ and R² for a compound corresponds in each case to one row of Table A

### Table 157

Compounds of the formula I.a in which R¹ and R⁴ are Cl, L′ is CH₃, L″ is F, L¹ is —O—CH₂CH₂—NHCH₃ and R² for a compound corresponds in each case to one row of Table A

### Table 158

Compounds of the formula I.a in which R¹ and R⁴ are Cl, L′ is CH₃, L″ is F, L¹ is —O—CH₂CH₂—N(CH₃)₂ and R² for a compound corresponds in each case to one row of Table A

### Table 159

Compounds of the formula I.a in which R¹ and R⁴ are Cl, L′ is CH₃, L″ is F, L¹ is —O—CH₂CH₂—NHC₂H₅ and R² for a compound corresponds in each case to one row of Table A

### Table 160

Compounds of the formula I.a in which R¹ and R⁴ are Cl, L′ is CH₃, L″ is F, L¹ is —O—CH₂CH₂—N(C₂H₅)₂ and R² for a compound corresponds in each case to one row of Table A

### Table 161

Compounds of the formula I.a in which R¹ and R⁴ are Cl, L′ is CH₃, L″ is F, L¹ is —O—CH₂CH₂CH₂—NHCH₃ and R² for a compound corresponds in each case to one row of Table A

### Table 162

Compounds of the formula I.a in which R¹ and R⁴ are Cl, L′ is CH₃, L″ is F, L¹ is —O—CH₂CH₂CH₂—N(CH₃)₂ and R² for a compound corresponds in each case to one row of Table A

### Table 163

Compounds of the formula I.a in which R¹ and R⁴ are Cl, L′ is CH₃, L″ is F, L¹ is —O—CH₂CH₂CH₂—NHC₂H₅ and R² for a compound corresponds in each case to one row of Table A

### Table 164

Compounds of the formula I.a in which R¹ and R⁴ are Cl, L′ is CH₃, L″ is F, L¹ is —O—CH₂CH₂CH₂—N(C₂H₅)₂ and R² for a compound corresponds in each case to one row of Table A

### Table 165

Compounds of the formula I.a in which R¹ and R⁴ are Cl, L′ is CH₃, L″ is F, L¹ is —O—CH₂CH₂CH₂CH₂—NHCH₃ and R² for a compound corresponds in each case to one row of Table A

### Table 166

Compounds of the formula I.a in which R¹ and R⁴ are Cl, L′ is CH₃, L″ is F, L¹ is —O—CH₂CH₂CH₂CH₂—N(CH₃)₂ and R² for a compound corresponds in each case to one row of Table A

### Table 167

Compounds of the formula I.a in which R¹ and R⁴ are Cl, L′ is CH₃, L″ is F, L¹ is —O—CH₂CH₂CH₂CH₂—NHC₂H₅ and R² for a compound corresponds in each case to one row of Table A

### Table 168

Compounds of the formula I.a in which R¹ and R⁴ are Cl, L′ is CH₃, L″ is F, L¹ is —O—CH₂CH₂CH₂CH₂—N(C₂H₅)₂ and R² for a compound corresponds in each case to one row of Table A

### Table 169

Compounds of the formula I.a in which R¹, R⁴, L′ and L″ are Cl, L¹ is —(OCH₂)₂—OH and R² for a compound corresponds in each case to one row of Table A

### Table 170

Compounds of the formula I.a in which R¹, R⁴, L′ and L″ are Cl, L¹ is —(OCH₂)₂—OCH₃ and R² for a compound corresponds in each case to one row of Table A

### Table 171

Compounds of the formula I.a in which R¹, R⁴, L′ and L″ are Cl, L¹ is —(OCH₂)₂—OC₂H₅ and R² for a compound corresponds in each case to one row of Table A

### Table 172

Compounds of the formula I.a in which R¹, R⁴, L′ and L″ are Cl, L¹ is —(OCH₂)₃—OH and R² for a compound corresponds in each case to one row of Table A

### Table 173

Compounds of the formula I.a in which R¹, R⁴, L′ and L″ are Cl, L¹ is —(OCH₂)₃—OCH₃ and R² for a compound corresponds in each case to one row of Table A

### Table 174

Compounds of the formula I.a in which R¹, R⁴, L′ and L″ are Cl, L¹ is —(OCH₂)₃—OC₂H₅ and R² for a compound corresponds in each case to one row of Table A

### Table 175

Compounds of the formula I.a in which R¹, R⁴, L′ and L″ are Cl, L¹ is —O—CH₂CH₂—OH and R² for a compound corresponds in each case to one row of Table A

### Table 176

Compounds of the formula I.a in which R¹, R⁴, L′ and L″ are Cl, L¹ is —O—CH₂CH₂—OCH₃ and R² for a compound corresponds in each case to one row of Table A

### Table 177

Compounds of the formula I.a in which R¹, R⁴, L′ and L″ are Cl, L¹ is —O—CH₂CH₂—OC₂H₅ and R² for a compound corresponds in each case to one row of Table A

### Table 178

Compounds of the formula I.a in which R¹, R⁴, L′ and L″ are Cl, L¹ is —(O—CH₂CH₂)₂—OH and R² for a compound corresponds in each case to one row of Table A

### Table 179

Compounds of the formula I.a in which R¹, R⁴, L′ and L″ are Cl, L¹ is —(O—CH₂CH₂)₂—OCH₃ and R² for a compound corresponds in each case to one row of Table A

### Table 180

Compounds of the formula I.a in which R¹, R⁴, L′ and L″ are Cl, L¹ is —(O—CH₂CH₂)₂—OC₂H₅ and R² for a compound corresponds in each case to one row of Table A

### Table 181

Compounds of the formula I.a in which R¹, R⁴, L′ and L″ are Cl, L¹ is —O—CH₂CH₂—NHCH₃ and R² for a compound corresponds in each case to one row of Table A

### Table 182

Compounds of the formula I.a in which R¹, R⁴, L′ and L″ are Cl, L¹ is —O—CH₂CH₂—N(CH₃)₂ and R² for a compound corresponds in each case to one row of Table A

### Table 183

Compounds of the formula I.a in which R¹, R⁴, L′ and L″ are Cl, L¹ is —O—CH₂CH₂—NHC₂H₅ and R² for a compound corresponds in each case to one row of Table A

### Table 184

Compounds of the formula I.a in which R¹, R⁴, L′ and L″ are Cl, L¹ is —O—CH₂CH₂—N(C₂H₅)₂ and R² for a compound corresponds in each case to one row of Table A

### Table 185

Compounds of the formula I.a in which R¹, R⁴, L′ and L″ are Cl, L¹ is —O—CH₂CH₂CH₂—NHCH₃ and R² for a compound corresponds in each case to one row of Table A

### Table 186

Compounds of the formula I.a in which R¹, R⁴, L′ and L″ are Cl, L¹ is —O—CH₂CH₂CH₂—N(CH₃)₂ and R² for a compound corresponds in each case to one row of Table A

### Table 187

Compounds of the formula I.a in which R¹, R⁴, L′ and L″ are Cl, L¹ is —O—CH₂CH₂CH₂—NHC₂H₅ and R² for a compound corresponds in each case to one row of Table A

### Table 188

Compounds of the formula I.a in which R¹, R⁴, L′ and L″ are Cl, L¹ is —O—CH₂CH₂CH₂—N(C₂H₅)₂ and R² for a compound corresponds in each case to one row of Table A

### Table 189

Compounds of the formula I.a in which R¹, R⁴, L′ and L″ are Cl, L¹ is —O—CH₂CH₂CH₂CH₂—NHCH₃ and R² for a compound corresponds in each case to one row of Table A

### Table 190

Compounds of the formula I.a in which R¹, R⁴, L′ and L″ are Cl, L¹ is —O—CH₂CH₂CH₂CH₂—N(CH₃)₂ and R² for a compound corresponds in each case to one row of Table A

### Table 191

Compounds of the formula I.a in which R¹, R⁴, L′ and L″ are Cl, L¹ is —O—CH₂CH₂CH₂CH₂—NHC₂H₅ and R² for a compound corresponds in each case to one row of Table A

### Table 192

Compounds of the formula I.a in which R¹, R⁴, L′ and L″ are Cl, L¹ is —O—CH₂CH₂CH₂CH₂—N(C₂H₅)₂ and R² for a compound corresponds in each case to one row of Table A

### Table 193

Compounds of the formula I.a in which R¹, R⁴ and L′ are Cl, L″ is CH₃, L¹ is —(OCH₂)₂—OH and R² for a compound corresponds in each case to one row of Table A

### Table 194

Compounds of the formula I.a in which R¹, R⁴ and L′ are Cl, L″ is CH₃, L¹ is —(OCH₂)₂—OCH₃ and R² for a compound corresponds in each case to one row of Table A

### Table 195

Compounds of the formula I.a in which R¹, R⁴ and L′ are Cl, L″ is CH₃, L¹ is —(OCH₂)₂—OC₂H₅ and R² for a compound corresponds in each case to one row of Table A

### Table 196

Compounds of the formula I.a in which R¹, R⁴ and L′ are Cl, L″ is CH₃, L¹ is —(OCH₂)₃—OH and R² for a compound corresponds in each case to one row of Table A

### Table 197

Compounds of the formula I.a in which R¹, R⁴ and L′ are Cl, L″ is CH₃, L¹ is —(OCH₂)₃—OCH₃ and R² for a compound corresponds in each case to one row of Table A

### Table 198

Compounds of the formula I.a in which R¹, R⁴ and L′ are Cl, L″ is CH₃, L¹ is —(OCH₂)₃—OC₂H₅ and R² for a compound corresponds in each case to one row of Table A

### Table 199

Compounds of the formula I.a in which R¹, R⁴ and L′ are Cl, L″ is CH₃, L¹ is —O—CH₂CH₂—OH and R² for a compound corresponds in each case to one row of Table A

### Table 200

Compounds of the formula I.a in which R¹, R⁴ and L′ are Cl, L″ is CH₃, L¹ is —O—CH₂CH₂—OCH₃ and R² for a compound corresponds in each case to one row of Table A

### Table 201

Compounds of the formula I.a in which R¹, R⁴ and L′ are Cl, L″ is CH₃, L¹ is —O—CH₂CH₂—OC₂H₅ and R² for a compound corresponds in each case to one row of Table A

### Table 202

Compounds of the formula I.a in which R¹, R⁴ and L′ are Cl, L″ is CH₃, L¹ is —(O—CH₂CH₂)₂—OH and R² for a compound corresponds in each case to one row of Table A

### Table 203

Compounds of the formula I.a in which R¹, R⁴ and L′ are Cl, L″ is CH₃, L¹ is —(O—CH₂CH₂)₂—OCH₃ and R² for a compound corresponds in each case to one row of Table A

### Table 204

Compounds of the formula I.a in which R¹, R⁴ and L′ are Cl, L″ is CH₃, L¹ is —(O—CH₂CH₂)₂—OC₂H₅ and R² for a compound corresponds in each case to one row of Table A

### Table 205

Compounds of the formula I.a in which R¹, R⁴ and L′ are Cl, L″ is CH₃, L¹ is —O—CH₂CH₂—NHCH₃ and R² for a compound corresponds in each case to one row of Table A

### Table 206

Compounds of the formula I.a in which R¹, R⁴ and L′ are Cl, L″ is CH₃, L¹ is —O—CH₂CH₂—N(CH₃)₂ and R² for a compound corresponds in each case to one row of Table A

### Table 207

Compounds of the formula I.a in which R¹, R⁴ and L′ are Cl, L″ is CH₃, L¹ is —O—CH₂CH₂—NHC₂H₅ and R² for a compound corresponds in each case to one row of Table A

### Table 208

Compounds of the formula I.a in which R¹, R⁴ and L′ are Cl, L″ is CH₃, L¹ is —O—CH₂CH₂—N(C₂H₅)₂ and R² for a compound corresponds in each case to one row of Table A

### Table 209

Compounds of the formula I.a in which R¹, R⁴ and L′ are Cl, L″ is CH₃, L¹ is —O—CH₂CH₂CH₂—NHCH₃ and R² for a compound corresponds in each case to one row of Table A

### Table 210

Compounds of the formula I.a in which R¹, R⁴ and L′ are Cl, L″ is CH₃, L¹ is —O—CH₂CH₂CH₂—N(CH₃)₂ and R² for a compound corresponds in each case to one row of Table A

### Table 211

Compounds of the formula I.a in which R¹, R⁴ and L′ are Cl, L″ is CH₃, L¹ is —O—CH₂CH₂CH₂—NHC₂H₅ and R² for a compound corresponds in each case to one row of Table A

### Table 212

Compounds of the formula I.a in which R¹, R⁴ and L′ are Cl, L″ is CH₃, L¹ is —O—CH₂CH₂CH₂—N(C₂H₅)₂ and R² for a compound corresponds in each case to one row of Table A

### Table 213

Compounds of the formula I.a in which R¹, R⁴ and L′ are Cl, L″ is CH₃, L¹ is —O—CH₂CH₂CH₂CH₂—NHCH₃ and R² for a compound corresponds in each case to one row of Table A

### Table 214

Compounds of the formula I.a in which R¹, R⁴ and L′ are Cl, L″ is CH₃, L¹ is —O—CH₂CH₂CH₂CH₂—N(CH₃)₂ and R² for a compound corresponds in each case to one row of Table A

### Table 215

Compounds of the formula I.a in which R¹, R⁴ and L′ are Cl, L″ is CH₃, L¹ is —O—CH₂CH₂CH₂CH₂—NHC₂H₅ and R² for a compound corresponds in each case to one row of Table A

### Table 216

Compounds of the formula I.a in which R¹, R⁴ and L′ are Cl, L″ is CH₃, L¹ is —O—CH₂CH₂CH₂CH₂—N(C₂H₅)₂ and R² for a compound corresponds in each case to one row of Table A

### Table 217

Compounds of the formula I.a in which R¹ and R⁴ are Cl, L′ and L″ are CH₃, L¹ is —(OCH₂)₂—OH and R² for a compound corresponds in each case to one row of Table A

### Table 218

Compounds of the formula I.a in which R¹ and R⁴ are Cl, L′ and L″ are CH₃, L¹ is —(OCH₂)₂—OCH₃ and R² for a compound corresponds in each case to one row of Table A

### Table 219

Compounds of the formula I.a in which R¹ and R⁴ are Cl, L′ and L″ are CH₃, L¹ is —(OCH₂)₂—OC₂H₅ and R² for a compound corresponds in each case to one row of Table A

### Table 220

Compounds of the formula I.a in which R¹ and R⁴ are Cl, L′ and L″ are CH₃, L¹ is —(OCH₂)₃—OH and R² for a compound corresponds in each case to one row of Table A

### Table 221

Compounds of the formula I.a in which R¹ and R⁴ are Cl, L′ and L″ are CH₃, L¹ is —(OCH₂)₃—OCH₃ and R² for a compound corresponds in each case to one row of Table A

### Table 222

Compounds of the formula I.a in which R¹ and R⁴ are Cl, L′ and L″ are CH₃, L¹ is —(OCH₂)₃—OC₂H₅ and R² for a compound corresponds in each case to one row of Table A

### Table 223

Compounds of the formula I.a in which R¹ and R⁴ are Cl, L′ and L″ are CH₃, L¹ is —O—CH₂CH₂—OH and R² for a compound corresponds in each case to one row of Table A

### Table 224

Compounds of the formula I.a in which R¹ and R⁴ are Cl, L′ and L″ are CH₃, L¹ is —O—CH₂CH₂—OCH₃ and R² for a compound corresponds in each case to one row of Table A

### Table 225

Compounds of the formula I.a in which R¹ and R⁴ are Cl, L′ and L″ are CH₃, L¹ is —O—CH₂CH₂—OC₂H₅ and R² for a compound corresponds in each case to one row of Table A

### Table 226

Compounds of the formula I.a in which R¹ and R⁴ are Cl, L′ and L″ are CH₃, L¹ is —(O—CH₂CH₂)₂—OH and R² for a compound corresponds in each case to one row of Table A

### Table 227

Compounds of the formula I.a in which R¹ and R⁴ are Cl, L′ and L″ are CH₃, L¹ is —(O—CH₂CH₂)₂—OCH₃ and R² for a compound corresponds in each case to one row of Table A

### Table 228

Compounds of the formula I.a in which R¹ and R⁴ are Cl, L′ and L″ are CH₃, L¹ is —(O—CH₂CH₂)₂—OC₂H₅ and R² for a compound corresponds in each case to one row of Table A

### Table 229

Compounds of the formula I.a in which R¹ and R⁴ are Cl, L′ and L″ are CH₃, L¹ is —O—CH₂CH₂—NHCH₃ and R² for a compound corresponds in each case to one row of Table A

### Table 230

Compounds of the formula I.a in which R¹ and R⁴ are Cl, L′ and L″ are CH₃, L¹ is —O—CH₂CH₂—N(CH₃)₂ and R² for a compound corresponds in each case to one row of Table A

### Table 231

Compounds of the formula I.a in which R¹ and R⁴ are Cl, L′ and L″ are CH₃, L¹ is —O—CH₂CH₂—NHC₂H₅ and R² for a compound corresponds in each case to one row of Table A

### Table 232

Compounds of the formula I.a in which R¹ and R⁴ are Cl, L′ and L″ are CH₃, L¹ is —O—CH₂CH₂—N(C₂H₅)₂ and R² for a compound corresponds in each case to one row of Table A

### Table 233

Compounds of the formula I.a in which R¹ and R⁴ are Cl, L′ and L″ are CH₃, L¹ is —O—CH₂CH₂CH₂—NHCH₃ and R² for a compound corresponds in each case to one row of Table A

### Table 234

Compounds of the formula I.a in which R¹ and R⁴ are Cl, L′ and L″ are CH₃, L¹ is —O—CH₂CH₂CH₂—N(CH₃)₂ and R² for a compound corresponds in each case to one row of Table A

### Table 235

Compounds of the formula I.a in which R¹ and R⁴ are Cl, L′ and L″ are CH₃, L¹ is —O—CH₂CH₂CH₂—NHC₂H₅ and R² for a compound corresponds in each case to one row of Table A

### Table 236

Compounds of the formula I.a in which R¹ and R⁴ are Cl, L′ and L″ are CH₃, L¹ is —O—CH₂CH₂CH₂—N(C₂H₅)₂ and R² for a compound corresponds in each case to one row of Table A

### Table 237

Compounds of the formula I.a in which R¹ and R⁴ are Cl, L′ and L″ are CH₃, L¹ is —O—CH₂CH₂CH₂CH₂—NHCH₃ and R² for a compound corresponds in each case to one row of Table A

### Table 238

Compounds of the formula I.a in which R¹ and R⁴ are Cl, L′ and L″ are CH₃, L¹ is —O—CH₂CH₂CH₂CH₂—N(CH₃)₂ and R² for a compound corresponds in each case to one row of Table A

### Table 239

Compounds of the formula I.a in which R¹ and R⁴ are Cl, L′ and L″ are CH₃, L¹ is —O—CH₂CH₂CH₂CH₂—NHC₂H₅ and R² for a compound corresponds in each case to one row of Table A

### Table 240

Compounds of the formula I.a in which R¹ and R⁴ are Cl, L′ and L″ are CH₃, L¹ is —O—CH₂CH₂CH₂CH₂—N(C₂H₅)₂ and R² for a compound corresponds in each case to one row of Table A

### Table 241

Compounds of the formula I.b in which R¹ and R⁴ are Cl, L′ is H, L¹ is —(OCH₂)₂—OH and R² for a compound corresponds in each case to one row of Table A

### Table 242

Compounds of the formula I.b in which R¹ and R⁴ are Cl, L′ is H, L¹ is —(OCH₂)₂—OCH₃ and R² for a compound corresponds in each case to one row of Table A

### Table 243

Compounds of the formula I.b in which R¹ and R⁴ are Cl, L′ is H, L¹ is —(OCH₂)₂—OC₂H₅ and R² for a compound corresponds in each case to one row of Table A

### Table 244

Compounds of the formula I.b in which R¹ and R⁴ are Cl, L′ is H, L¹ is —(OCH₂)₃—OH and R² for a compound corresponds in each case to one row of Table A

### Table 245

Compounds of the formula I.b in which R¹ and R⁴ are Cl, L′ is H, L¹ is —(OCH₂)₃—OCH₃ and R² for a compound corresponds in each case to one row of Table A

### Table 246

Compounds of the formula I.b in which R¹ and R⁴ are Cl, L′ is H, L¹ is —(OCH₂)₃—OC₂H₅ and R² for a compound corresponds in each case to one row of Table A

### Table 247

Compounds of the formula I.b in which R¹ and R⁴ are Cl, L′ is H, L¹ is —O—CH₂CH₂—OH and R² for a compound corresponds in each case to one row of Table A

### Table 248

Compounds of the formula I.b in which R¹ and R⁴ are Cl, L′ is H, L¹ is —O—CH₂CH₂—OCH₃ and R² for a compound corresponds in each case to one row of Table A

### Table 249

Compounds of the formula I.b in which R¹ and R⁴ are Cl, L′ is H, L¹ is —O—CH₂CH₂—OC₂H₅ and R² for a compound corresponds in each case to one row of Table A

### Table 250

Compounds of the formula I.b in which R¹ and R⁴ are Cl, L′ is H, L¹ is —(O—CH₂CH₂)₂—OH and R² for a compound corresponds in each case to one row of Table A

### Table 251

Compounds of the formula I.b in which R¹ and R⁴ are Cl, L′ is H, L¹ is —(O—CH₂CH₂)₂—OCH₃ and R² for a compound corresponds in each case to one row of Table A

### Table 252

Compounds of the formula I.b in which R¹ and R⁴ are Cl, L′ is H, L¹ is —(O—CH₂CH₂)₂—OC₂H₅ and R² for a compound corresponds in each case to one row of Table A

### Table 253

Compounds of the formula I.b in which R¹ and R⁴ are Cl, L′ is H, L¹ is —O—CH₂CH₂—NHCH₃ and R² for a compound corresponds in each case to one row of Table A

### Table 254

Compounds of the formula I.b in which R¹ and R⁴ are Cl, L′ is H, L¹ is —O—CH₂CH₂—N(CH₃)₂ and R² for a compound corresponds in each case to one row of Table A

### Table 255

Compounds of the formula I.b in which R¹ and R⁴ are Cl, L′ is H, L¹ is —O—CH₂CH₂—NHC₂H₅ and R² for a compound corresponds in each case to one row of Table A

### Table 256

Compounds of the formula I.b in which R¹ and R⁴ are Cl, L′ is H, L¹ is —O—CH₂CH₂—N(C₂H₅)₂ and R² for a compound corresponds in each case to one row of Table A

### Table 257

Compounds of the formula I.b in which R¹ and R⁴ are Cl, L′ is H, L¹ is —O—CH₂CH₂CH₂—NHCH₃ and R² for a compound corresponds in each case to one row of Table A

### Table 258

Compounds of the formula I.b in which R¹ and R⁴ are Cl, L′ is H, L¹ is —O—CH₂CH₂CH₂—N(CH₃)₂ and R² for a compound corresponds in each case to one row of Table A

### Table 259

Compounds of the formula I.b in which R¹ and R⁴ are Cl, L′ is H, L¹ is —O—CH₂CH₂CH₂—NHC₂H₅ and R² for a compound corresponds in each case to one row of Table A

### Table 260

Compounds of the formula I.b in which R¹ and R⁴ are Cl, L′ is H, L¹ is —O—CH₂CH₂CH₂—N(C₂H₅)₂ and R² for a compound corresponds in each case to one row of Table A

### Table 261

Compounds of the formula I.b in which R¹ and R⁴ are Cl, L′ is H, L¹ is —O—CH₂CH₂CH₂CH₂—NHCH₃ and R² for a compound corresponds in each case to one row of Table A

### Table 262

Compounds of the formula I.b in which R¹ and R⁴ are Cl, L′ is H, L¹ is —O—CH₂CH₂CH₂CH₂—N(CH₃)₂ and R² for a compound corresponds in each case to one row of Table A

### Table 263

Compounds of the formula I.b in which R¹ and R⁴ are Cl, L′ is H, L¹ is —O—CH₂CH₂CH₂CH₂—NHC₂H₅ and R² for a compound corresponds in each case to one row of Table A

### Table 264

Compounds of the formula I.b in which R¹ and R⁴ are Cl, L′ is H, L¹ is —O—CH₂CH₂CH₂CH₂—N(C₂H₅)₂ and R² for a compound corresponds in each case to one row of Table A

### Table 265

Compounds of the formula I.b in which R¹, R⁴ and L′ are Cl, L¹ is —(OCH₂)₂—OH and R² for a compound corresponds in each case to one row of Table A

### Table 266

Compounds of the formula I.b in which R¹, R⁴ and L′ are Cl, L¹ is —(OCH₂)₂—OCH₃ and R² for a compound corresponds in each case to one row of Table A

### Table 267

Compounds of the formula I.b in which R¹, R⁴ and L′ are Cl, L¹ is —(OCH₂)₂—OC₂H₅ and R² for a compound corresponds in each case to one row of Table A

### Table 268

Compounds of the formula I.b in which R¹, R⁴ and L′ are Cl, L¹ is —(OCH₂)₃—OH and R² for a compound corresponds in each case to one row of Table A

### Table 269

Compounds of the formula I.b in which R¹, R⁴ and L′ are Cl, L¹ is —(OCH₂)₃—OCH₃ and R² for a compound corresponds in each case to one row of Table A

### Table 270

Compounds of the formula I.b in which R¹, R⁴ and L′ are Cl, L¹ is —(OCH₂)₃—OC₂H₅ and R² for a compound corresponds in each case to one row of Table A

### Table 271

Compounds of the formula I.b in which R¹, R⁴ and L′ are Cl, L¹ is —O—CH₂CH₂—OH and R² for a compound corresponds in each case to one row of Table A

### Table 272

Compounds of the formula I.b in which R¹, R⁴ and L′ are Cl, L¹ is —O—CH₂CH₂—OCH₃ and R² for a compound corresponds in each case to one row of Table A

### Table 273

Compounds of the formula I.b in which R¹, R⁴ and L′ are Cl, L¹ is —O—CH₂CH₂—OC₂H₅ and R² for a compound corresponds in each case to one row of Table A

### Table 274

Compounds of the formula I.b in which R¹, R⁴ and L′ are Cl, L¹ is —(O—CH₂CH₂)₂—OH and R² for a compound corresponds in each case to one row of Table A

### Table 275

Compounds of the formula I.b in which R¹, R⁴ and L′ are Cl, L¹ is —(O—CH₂CH₂)₂—OCH₃ and R² for a compound corresponds in each case to one row of Table A

### Table 276

Compounds of the formula I.b in which R¹, R⁴ and L′ are Cl, L¹ is —(O—CH₂CH₂)₂—OC₂H₅ and R² for a compound corresponds in each case to one row of Table A

### Table 277

Compounds of the formula I.b in which R¹, R⁴ and L′ are Cl, L¹ is —O—CH₂CH₂—NHCH₃ and R² for a compound corresponds in each case to one row of Table A

### Table 278

Compounds of the formula I.b in which R¹, R⁴ and L′ are Cl, L¹ is —O—CH₂CH₂—N(CH₃)₂ and R² for a compound corresponds in each case to one row of Table A

### Table 279

Compounds of the formula I.b in which R¹, R⁴ and L′ are Cl, L¹ is —O—CH₂CH₂—NHC₂H₅ and R² for a compound corresponds in each case to one row of Table A

### Table 280

Compounds of the formula I.b in which R¹, R⁴ and L′ are Cl, L¹ is —O—CH₂CH₂—N(C₂H₅)₂ and R² for a compound corresponds in each case to one row of Table A

### Table 281

Compounds of the formula I.b in which R¹, R⁴ and L′ are Cl, L¹ is —O—CH₂CH₂CH₂—NHCH₃ and R² for a compound corresponds in each case to one row of Table A

### Table 282

Compounds of the formula I.b in which R¹, R⁴ and L′ are Cl, L¹ is —O—CH₂CH₂CH₂—N(CH₃)₂ and R² for a compound corresponds in each case to one row of Table A

### Table 283

Compounds of the formula I.b in which R¹, R⁴ and L′ are Cl, L¹ is —O—CH₂CH₂CH₂—NHC₂H₅ and R² for a compound corresponds in each case to one row of Table A

### Table 284

Compounds of the formula I.b in which R¹, R⁴ and L′ are Cl, L¹ is —O—CH₂CH₂CH₂—N(C₂H₅)₂ and R² for a compound corresponds in each case to one row of Table A

### Table 285

Compounds of the formula I.b in which R¹, R⁴ and L′ are Cl, L¹ is —O—CH₂CH₂CH₂CH₂—NHCH₃ and R² for a compound corresponds in each case to one row of Table A

### Table 286

Compounds of the formula I.b in which R¹, R⁴ and L′ are Cl, L¹ is —O—CH₂CH₂CH₂CH₂—N(CH₃)₂ and R² for a compound corresponds in each case to one row of Table A

### Table 287

Compounds of the formula I.b in which R¹, R⁴ and L′ are Cl, L¹ is —O—CH₂CH₂CH₂CH₂—NHC₂H₅ and R² for a compound corresponds in each case to one row of Table A

### Table 288

Compounds of the formula I.b in which R¹, R⁴ and L′ are Cl, L¹ is —O—CH₂CH₂CH₂CH₂—N(C₂H₅)₂ and R² for a compound corresponds in each case to one row of Table A

### Table 289

Compounds of the formula I.b in which R¹ and R⁴ are Cl, L′ is F, L¹ is —(OCH₂)₂—OH and R² for a compound corresponds in each case to one row of Table A

### Table 290

Compounds of the formula I.b in which R¹ and R⁴ are Cl, L′ is F, L¹ is —(OCH₂)₂—OCH₃ and R² for a compound corresponds in each case to one row of Table A

### Table 291

Compounds of the formula I.b in which R¹ and R⁴ are Cl, L′ is F, L¹ is —(OCH₂)₂—OC₂H₅ and R² for a compound corresponds in each case to one row of Table A

### Table 292

Compounds of the formula I.b in which R¹ and R⁴ are Cl, L′ is F, L¹ is —(OCH₂)₃—OH and R² for a compound corresponds in each case to one row of Table A

### Table 293

Compounds of the formula I.b in which R¹ and R⁴ are Cl, L′ is F, L¹ is —(OCH₂)₃—OCH₃ and R² for a compound corresponds in each case to one row of Table A

### Table 294

Compounds of the formula I.b in which R¹ and R⁴ are Cl, L′ is F, L¹ is —(OCH₂)₃—OC₂H₅ and R² for a compound corresponds in each case to one row of Table A

### Table 295

Compounds of the formula I.b in which R¹ and R⁴ are Cl, L′ is F, L¹ is —O—CH₂CH₂—OH and R² for a compound corresponds in each case to one row of Table A

### Table 296

Compounds of the formula I.b in which R¹ and R⁴ are Cl, L′ is F, L¹ is —O—CH₂CH₂—OCH₃ and R² for a compound corresponds in each case to one row of Table A

### Table 297

Compounds of the formula I.b in which R¹ and R⁴ are Cl, L′ is F, L¹ is —O—CH₂CH₂—OC₂H₅ and R² for a compound corresponds in each case to one row of Table A

### Table 298

Compounds of the formula I.b in which R¹ and R⁴ are Cl, L′ is F, L¹ is —(O—CH₂CH₂)₂—OH and R² for a compound corresponds in each case to one row of Table A

### Table 299

Compounds of the formula I.b in which R¹ and R⁴ are Cl, L′ is F, L¹ is —(O—CH₂CH₂)₂—OCH₃ and R² for a compound corresponds in each case to one row of Table A

### Table 300

Compounds of the formula I.b in which R¹ and R⁴ are Cl, L′ is F, L¹ is —(O—CH₂CH₂)₂—OC₂H₅ and R² for a compound corresponds in each case to one row of Table A

### Table 301

Compounds of the formula I.b in which R¹ and R⁴ are Cl, L′ is F, L¹ is —O—CH₂CH₂—NHCH₃ and R² for a compound corresponds in each case to one row of Table A

### Table 302

Compounds of the formula I.b in which R¹ and R⁴ are Cl, L′ is F, L¹ is —O—CH₂CH₂—N(CH₃)₂ and R² for a compound corresponds in each case to one row of Table A

### Table 303

Compounds of the formula I.b in which R¹ and R⁴ are Cl, L′ is F, L¹ is —O—CH₂CH₂—NHC₂H₅ and R² for a compound corresponds in each case to one row of Table A

### Table 304

Compounds of the formula I.b in which R¹ and R⁴ are Cl, L′ is F, L¹ is —O—CH₂CH₂—N(C₂H₅)₂ and R² for a compound corresponds in each case to one row of Table A

### Table 305

Compounds of the formula I.b in which R¹ and R⁴ are Cl, L′ is F, L¹ is —O—CH₂CH₂CH₂—NHCH₃ and R² for a compound corresponds in each case to one row of Table A

### Table 306

Compounds of the formula I.b in which R¹ and R⁴ are Cl, L′ is F, L¹ is —O—CH₂CH₂CH₂—N(CH₃)₂ and R² for a compound corresponds in each case to one row of Table A

### Table 307

Compounds of the formula I.b in which R¹ and R⁴ are Cl, L′ is F, L¹ is —O—CH₂CH₂CH₂—NHC₂H₅ and R² for a compound corresponds in each case to one row of Table A

### Table 308

Compounds of the formula I.b in which R¹ and R⁴ are Cl, L′ is F, L¹ is —O—CH₂CH₂CH₂—N(C₂H₅)₂ and R² for a compound corresponds in each case to one row of Table A

### Table 309

Compounds of the formula I.b in which R¹ and R⁴ are Cl, L′ is F, L¹ is —O—CH₂CH₂CH₂CH₂—NHCH₃ and R² for a compound corresponds in each case to one row of Table A

### Table 310

Compounds of the formula I.b in which R¹ and R⁴ are Cl, L′ is F, L¹ is —O—CH₂CH₂CH₂CH₂—N(CH₃)₂ and R² for a compound corresponds in each case to one row of Table A

### Table 311

Compounds of the formula I.b in which R¹ and R⁴ are Cl, L′ is F, L¹ is —O—CH₂CH₂CH₂CH₂—NHC₂H₅ and R² for a compound corresponds in each case to one row of Table A

### Table 312

Compounds of the formula I.b in which R¹ and R⁴ are Cl, L′ is F, L¹ is —O—CH₂CH₂CH₂CH₂—N(C₂H₅)₂ and R² for a compound corresponds in each case to one row of Table A

### Table 313

Compounds of the formula I.b in which R¹ and R⁴ are Cl, L′ is CH₃, L¹ is —(OCH₂)₂—OH and R² for a compound corresponds in each case to one row of Table A

### Table 314

Compounds of the formula I.b in which R¹ and R⁴ are Cl, L′ is CH₃, L¹ is —(OCH₂)₂—OCH₃ and R² for a compound corresponds in each case to one row of Table A

### Table 315

Compounds of the formula I.b in which R¹ and R⁴ are Cl, L′ is CH₃, L¹ is —(OCH₂)₂—OC₂H₅ and R² for a compound corresponds in each case to one row of Table A

### Table 316

Compounds of the formula I.b in which R¹ and R⁴ are Cl, L′ is CH₃, L¹ is —(OCH₂)₃—OH and R² for a compound corresponds in each case to one row of Table A

### Table 317

Compounds of the formula I.b in which R¹ and R⁴ are Cl, L′ is CH₃, L¹ is —(OCH₂)₃—OCH₃ and R² for a compound corresponds in each case to one row of Table A

### Table 318

Compounds of the formula I.b in which R¹ and R⁴ are Cl, L′ is CH₃, L¹ is —(OCH₂)₃—OC₂H₅ and R² for a compound corresponds in each case to one row of Table A

### Table 319

Compounds of the formula I.b in which R¹ and R⁴ are Cl, L′ is CH₃, L¹ is —O—CH₂CH₂—OH and R² for a compound corresponds in each case to one row of Table A

### Table 320

Compounds of the formula I.b in which R¹ and R⁴ are Cl, L′ is CH₃, L¹ is —O—CH₂CH₂—OCH₃ and R² for a compound corresponds in each case to one row of Table A

### Table 321

Compounds of the formula I.b in which R¹ and R⁴ are Cl, L′ is CH₃, L¹ is —O—CH₂CH₂—OC₂H₅ and R² for a compound corresponds in each case to one row of Table A

### Table 322

Compounds of the formula I.b in which R¹ and R⁴ are Cl, L′ is CH₃, L¹ is —(O—CH₂CH₂)₂—OH and R² for a compound corresponds in each case to one row of Table A

### Table 323

Compounds of the formula I.b in which R¹ and R⁴ are Cl, L′ is CH₃, L¹ is —(O—CH₂CH₂)₂—OCH₃ and R² for a compound corresponds in each case to one row of Table A

### Table 324

Compounds of the formula I.b in which R¹ and R⁴ are Cl, L′ is CH₃, L¹ is —(O—CH₂CH₂)₂—OC₂H₅ and R² for a compound corresponds in each case to one row of Table A

### Table 325

Compounds of the formula I.b in which R¹ and R⁴ are Cl, L′ is CH₃, L¹ is —O—CH₂CH₂—NHCH₃ and R² for a compound corresponds in each case to one row of Table A

### Table 326

Compounds of the formula I.b in which R¹ and R⁴ are Cl, L′ is CH₃, L¹ is —O—CH₂CH₂—N(CH₃)₂ and R² for a compound corresponds in each case to one row of Table A

### Table 327

Compounds of the formula I.b in which R¹ and R⁴ are Cl, L′ is CH₃, L¹ is —O—CH₂CH₂—NHC₂H₅ and R² for a compound corresponds in each case to one row of Table A

### Table 328

Compounds of the formula I.b in which R¹ and R⁴ are Cl, L′ is CH₃, L¹ is —O—CH₂CH₂—N(C₂H₅)₂ and R² for a compound corresponds in each case to one row of Table A

### Table 329

Compounds of the formula I.b in which R¹ and R⁴ are Cl, L′ is CH₃, L¹ is —O—CH₂CH₂CH₂—NHCH₃ and R² for a compound corresponds in each case to one row of Table A

### Table 330

Compounds of the formula I.b in which R¹ and R⁴ are Cl, L′ is CH₃, L¹ is —O—CH₂CH₂CH₂—N(CH₃)₂ and R² for a compound corresponds in each case to one row of Table A

### Table 331

Compounds of the formula I.b in which R¹ and R⁴ are Cl, L′ is CH₃, L¹ is —O—CH₂CH₂CH₂—NHC₂H₅ and R² for a compound corresponds in each case to one row of Table A

### Table 332

Compounds of the formula I.b in which R¹ and R⁴ are Cl, L′ is CH₃, L¹ is —O—CH₂CH₂CH₂—N(C₂H₅)₂ and R² for a compound corresponds in each case to one row of Table A

### Table 333

Compounds of the formula I.b in which R¹ and R⁴ are Cl, L′ is CH₃, L¹ is —O—CH₂CH₂CH₂CH₂—NHCH₃ and R² for a compound corresponds in each case to one row of Table A

### Table 334

Compounds of the formula I.b in which R¹ and R⁴ are Cl, L′ is CH₃, L¹ is —O—CH₂CH₂CH₂CH₂—N(CH₃)₂ and R² for a compound corresponds in each case to one row of Table A

### Table 335

Compounds of the formula I.b in which R¹ and R⁴ are Cl, L′ is CH₃, L¹ is —O—CH₂CH₂CH₂CH₂—NHC₂H₅ and R² for a compound corresponds in each case to one row of Table A

### Table 336

Compounds of the formula I.b in which R¹ and R⁴ are Cl, L′ is CH₃, L¹ is —O—CH₂CH₂CH₂CH₂—N(C₂H₅)₂ and R² for a compound corresponds in each case to one row of Table A

TABLE A

| No. | R² |
|---|---|
| A-1 | CH₃ |
| A-2 | CH₂CH₃ |
| A-3 | CH₂CH₂CH₃ |
| A-4 | CH(CH₃)₂ |
| A-5 | CH₂CH(CH₃)₂ |
| A-6 | (±) CH(CH₃)CH₂CH₃ |
| A-7 | (S) CH(CH₃)CH₂CH₃ |
| A-8 | (R) CH(CH₃)CH₂CH₃ |
| A-9 | (CH₂)₃CH₃ |
| A-10 | C(CH₃)₃ |
| A-11 | (CH₂)₄CH₃ |
| A-12 | CH(CH₂CH₃)₂ |
| A-13 | CH₂CH₂CH(CH₃)₂ |
| A-14 | (±) CH(CH₃)(CH₂)₂CH₃ |
| A-15 | (S) CH(CH₃)(CH₂)₂CH₃ |
| A-16 | (R) CH(CH₃)(CH₂)₂CH₃ |
| A-17 | (±) CH₂CH(CH₃)CH₂CH₃ |
| A-18 | (S) CH₂CH(CH₃)CH₂CH₃ |
| A-19 | (R) CH₂CH(CH₃)CH₂CH₃ |
| A-20 | (±) CH(CH₃)CH(CH₃)₂ |
| A-21 | (S) CH(CH₃)CH(CH₃)₂ |
| A-22 | (R) CH(CH₃)CH(CH₃)₂ |
| A-23 | (CH₂)₅CH₃ |
| A-24 | (±,±) CH(CH₃)CH(CH₃)CH₂CH₃ |
| A-25 | (±,S) CH(CH₃)CH(CH₃)CH₂CH₃ |
| A-26 | (±,R) CH(CH₃)CH(CH₃)CH₂CH₃ |
| A-27 | (±) CH₂CH(CH₃)CF₃ |
| A-28 | (S) CH₂CH(CH₃)CF₃ |
| A-29 | (R) CH₂CH(CH₃)CF₃ |
| A-30 | (±) CH₂CH(CF₃)CH₂CH₃ |
| A-31 | (S) CH₂CH(CF₃)CH₂CH₃ |
| A-32 | (R) CH₂CH(CF₃)CH₂CH₃ |
| A-33 | (±,±) CH(CH₃)CH(CH₃)CF₃ |
| A-34 | (±,S) CH(CH₃)CH(CH₃)CF₃ |
| A-35 | (±,R) CH(CH₃)CH(CH₃)CF₃ |
| A-36 | (±,±) CH(CH₃)CH(CF₃)CH₂CH₃ |
| A-37 | (±,R) CH(CH₃)CH(CF₃)CH₂CH₃ |
| A-38 | (±,S) CH(CH₃)CH(CF₃)CH₂CH₃ |
| A-39 | CF₃ |
| A-40 | CF₂CF₃ |
| A-41 | CF₂CF₂CF₃ |
| A-42 | cyclo-C₃H₅ |
| A-43 | (1-CH₃)-cyclo-C₃H₄ |
| A-44 | cyclo-C₅H₉ |
| A-45 | cyclo-C₆H₁₁ |
| A-46 | (4-CH₃)-cyclo-C₆H₁₀ |
| A-47 | CH₂C(CH₃)═CH₂ |
| A-48 | CH₂CH₂C(CH₃)═CH₂ |
| A-49 | CH₂C(CH₃)₃ |
| A-50 | CH₂Si(CH₃)₃ |
| A-51 | n-C₆H₁₃ |
| A-52 | (CH₂)₃CH(CH₃)₂ |
| A-53 | (CH₂)₂CH(CH₃)C₂H₅ |
| A-54 | CH₂CH(CH₃)-n-C₃H₇ |
| A-55 | CH(CH₃)-n-C₄H₉ |
| A-56 | CH₂CH(C₂H₅)₂ |
| A-57 | CH(C₂H₅)-n-C₃H₇ |
| A-58 | CH₂-cyclo-C₅H₉ |
| A-59 | CH₂CH(CH₃)CH(CH₃)₂ |
| A-60 | CH(CH₃)CH₂CH(CH₃)₂ |
| A-61 | CH(CH₃)CH(CH₃)C₂H₅ |
| A-62 | CH(CH₃)C(CH₃)₃ |
| A-63 | (CH₂)₂C(CH₃)₃ |
| A-64 | CH₂—C(CH₃)₂C₂H₅ |
| A-65 | 2-CH₃-cyclo-C₅H₈ |
| A-66 | 3-CH₃-cyclo-C₅H₈ |
| A-67 | C(CH₃)₂-n-C₃H₇ |
| A-68 | (CH₂)₆CH₃ |
| A-69 | (CH₂)₄CH(CH₃)₂ |
| A-70 | (CH₂)₃CH(CH₃)C₂H₅ |
| A-71 | (CH₂)₂CH(CH₃)-n-C₃H₇ |
| A-72 | CH₂CH(CH₃)-n-C₄H₉ |
| A-73 | CH(CH₃)-n-C₅H₁₁ |
| A-74 | (CH₂)₃C(CH₃)₃ |
| A-75 | (CH₂)₂CH(CH₃)CH(CH₃)₂ |
| A-76 | (CH₂)CH(CH₃)CH₂CH(CH₃)₂ |
| A-77 | CH(CH₃)(CH₂)₂CH(CH₃)₂ |
| A-78 | (CH₂)₂C(CH₃)₂C₂H₅ |
| A-79 | CH₂CH(CH₃)CH(CH₃)C₂H₅ |
| A-80 | CH(CH₃)CH₂CH(CH₃)C₂H₅ |
| A-81 | CH₂C(CH₃)₂-n-C₃H₇ |
| A-82 | CH(CH₃)CH(CH₃)-n-C₃H₇ |
| A-83 | C(CH₃)₂-n-C₄H₉ |
| A-84 | (CH₂)₂CH(C₂H₅)₂ |
| A-85 | CH₂CH(C₂H₅)-n-C₃H₇ |
| A-86 | CH(C₂H₅)-n-C₄H₉ |
| A-87 | CH₂CH(CH₃)C(CH₃)₃ |
| A-88 | CH(CH₃)CH₂C(CH₃)₃ |
| A-89 | CH₂C(CH₃)₂CH(CH₃)₂ |
| A-90 | CH₂CH(C₂H₅)CH(CH₃)₂ |
| A-91 | CH(CH₃)CH(CH₃)CH(CH₃)₂ |
| A-92 | C(CH₃)₂CH₂CH(CH₃)₂ |
| A-93 | CH(C₂H₅)CH₂CH(CH₃)₂ |
| A-94 | CH(CH₃)C(CH₃)₂C₂H₅ |
| A-95 | CH(CH₃)CH(C₂H₅)₂ |
| A-96 | C(CH₃)₂CH(CH₃)C₂H₅ |
| A-97 | CH(C₂H₅)CH(CH₃)C₂H₅ |
| A-98 | C(CH₃)(C₂H₅)-n-C₃H₇ |
| A-99 | CH-(n-C₃H₇)₂ |
| A-100 | CH(n-C₃H₇)CH(CH₃)₂ |
| A-101 | C(CH₃)₂C(CH₃)₃ |
| A-102 | C(CH₃)(C₂H₅)CH(CH₃)₂ |
| A-103 | C(C₂H₅)₃ |
| A-104 | (3-CH₃)-cyclo-C₆H₁₀ |
| A-105 | (2-CH₃)-cyclo-C₆H₁₀ |
| A-106 | n-C₈H₁₇ |
| A-107 | CH₂C(═NO—CH₃)CH₃ |
| A-108 | CH₂C(═NO—C₂H₅)CH₃ |
| A-109 | CH₂C(═NO-n-C₃H₇)CH₃ |
| A-110 | CH₂C(═NO-i-C₃H₇)CH₃ |
| A-111 | CH(CH₃)C(═NOCH₃)CH₃ |
| A-112 | CH(CH₃)C(═NOC₂H₅)CH₃ |
| A-113 | CH(CH₃)C(═NO-n-C₃H₇)CH₃ |
| A-114 | CH(CH₃)C(═NO-i-C₃H₇)CH₃ |
| A-115 | C(═NOCH₃)C(═NOCH₃)CH₃ |
| A-116 | C(═NOCH₃)C(═NOC₂H₅)CH₃ |
| A-117 | C(═NOCH₃)C(═NO-n-C₃H₇)CH₃ |
| A-118 | C(═NOCH₃)C(═NO-i-C₃H₇)CH₃ |
| A-119 | C(═NOC₂H₅)C(═NOCH₃)CH₃ |
| A-120 | C(═NOC₂H₅)C(═NOC₂H₅)CH₃ |
| A-121 | C(═NOC₂H₅)C(═NO-n-C₃H₇)CH₃ |
| A-122 | C(═NOC₂H₅)C(═NO-i-C₃H₇)CH₃ |
| A-123 | CH₂C(═NO—CH₃)C₂H₅ |
| A-124 | CH₂C(═NO—C₂H₅)C₂H₅ |
| A-125 | CH₂C(═NO-n-C₃H₇)C₂H₅ |
| A-126 | CH₂C(═NO-i-C₃H₇)C₂H₅ |
| A-127 | CH(CH₃)C(═NOCH₃)C₂H₅ |
| A-128 | CH(CH₃)C(═NOC₂H₅)C₂H₅ |
| A-129 | CH(CH₃)C(═NO-n-C₃H₇)C₂H₅ |
| A-130 | CH(CH₃)C(═NO-i-C₃H₇)C₂H₅ |
| A-131 | C(═NOCH₃)C(═NOCH₃)C₂H₅ |
| A-132 | C(═NOCH₃)C(═NOC₂H₅)C₂H₅ |
| A-133 | C(═NOCH₃)C(═NO-n-C₃H₇)C₂H₅ |
| A-134 | C(═NOCH₃)C(═NO-i-C₃H₇)C₂H₅ |
| A-135 | C(═NOC₂H₅)C(═NOCH₃)C₂H₅ |
| A-136 | C(═NOC₂H₅)C(═NOC₂H₅)C₂H₅ |
| A-137 | C(═NOC₂H₅)C(═NO-n-C₃H₇)C₂H₅ |
| A-138 | C(═NOC₂H₅)C(═NO-i-C₃H₇)C₂H₅ |
| A-139 | CH═CHCH₂CH₃ |
| A-140 | CH₂CH═CHCH₃ |
| A-141 | CH₂CH₂CH═CH₂ |
| A-142 | C(CH₃)₂CH₂CH₃ |
| A-143 | CH═C(CH₃)₂ |
| A-144 | C(═CH₂)CH₂CH₃ |
| A-145 | C(CH₃)═CHCH₃ |
| A-146 | CH(CH₃)CH═CH₂ |
| A-147 | CH═CH-n-C₃H₇ |
| A-148 | CH₂—CH═CHC₂H₅ |
| A-149 | (CH₂)₂CH═CH—CH₃ |
| A-150 | (CH₂)₃CH═CH₂ |
| A-151 | CH═CHCH(CH₃)₂ |
| A-152 | CH₂CH═C(CH₃)₂ |
| A-153 | (CH₂)₂C(CH₃)═CH₂ |
| A-154 | CH═C(CH₃)C₂H₅ |
| A-155 | (CH₂)C(═CH₂)C₂H₅ |
| A-156 | (CH₂)C(CH₃)═CHCH₃ |
| A-157 | CH₂CH(CH₃)CH═CH₂ |
| A-158 | C(═CH₂)CH₂(CH₂)CH₃ |
| A-159 | C(CH₃)═CHCH₂CH₃ |
| A-160 | C(CH₃)CH═CHCH₃ |
| A-161 | C(CH₃)CH₂CH═CH₂ |
| A-162 | C(═CH₂)CH(CH₃)₂ |
| A-163 | C(CH₃)═C(CH₃)₂ |
| A-164 | CH(CH₃)C(═CH₂)CH₃ |
| A-165 | C(CH₃)₂CH═CH₂ |
| A-166 | C(C₂H₅)═CHCH₃ |
| A-167 | C(C₂H₅)CH═CH₂ |
| A-168 | CH═CHCH₂CH₂CH₂CH₃ |
| A-169 | CH₂CH═CHCH₂CH₂CH₃ |
| A-170 | CH₂CH₂CH═CHCH₂CH₃ |
| A-171 | CH₂CH₂CH₂CH═CHCH₃ |
| A-172 | CH₂CH₂CH₂CH₂CH═CH₂ |
| A-173 | CH═CHCH₂CH(CH₃)CH₃ |
| A-174 | CH₂CH═CHCH(CH₃)CH₃ |
| A-175 | CH₂CH₂CH═C(CH₃)CH₃ |
| A-176 | CH₂CH₂CH₂C(CH₃)═CH₂ |
| A-177 | CH═CHCH(CH₃)CH₂CH₃ |
| A-178 | CH₂CH═C(CH₃)CH₂CH₃ |
| A-179 | CH₂CH₂C(═CH₂)CH₂CH₃ |
| A-180 | CH₂CH₂C(CH₃)═CHCH₃ |
| A-181 | CH₂CH₂CH(CH₃)CH═CH₂ |
| A-182 | CH═C(CH₃)CH₂CH₂CH₃ |
| A-183 | CH₂C(═CH₂)CH₂CH₂CH₃ |
| A-184 | CH₂C(CH₃)═CHCH₂CH₃ |
| A-185 | CH₂CH(CH₃)CH═CHCH₃ |
| A-186 | CH₂CH(CH₃)CH₂CH═CH₂ |
| A-187 | C(═CH₂)CH₂CH₂CH₂CH₃ |
| A-188 | C(CH₃)═CH—CH₂CH₂CH₃ |
| A-189 | CH(CH₃)CH═CHCH₂CH₃ |
| A-190 | CH(CH₃)CH₂CH═CHCH₃ |
| A-191 | CH(CH₃)CH₂CH₂CH═CH₂ |
| A-192 | CH═CHC(CH₃)₃ |
| A-193 | CH═C(CH₃)CH(CH₃)CH₃ |
| A-194 | CH₂C(═CH₂)CH(CH₃)CH₃ |
| A-195 | CH₂C(CH₃)═C(CH₃)CH₃ |
| A-196 | CH₂CH(CH₃)C(═CH₂)CH₃ |
| A-197 | C(═CH₂)CH₂CH(CH₃)CH₃ |
| A-198 | C(CH₃)═CHCH(CH₃)CH₃ |
| A-199 | CH(CH₃)CH═C(CH₃)CH₃ |
| A-200 | CH(CH₃)CH₂C(═CH₂)CH₃ |
| A-201 | CH═C(CH₂CH₃)CH₂CH₃ |
| A-202 | CH₂C(═CHCH₃)CH₂CH₃ |
| A-203 | CH₂CH(CH═CH₂)CH₂CH₃ |
| A-204 | C(═CH—CH₃)—CH₂CH₂CH₃ |
| A-205 | CH(CH═CH₂)CH₂CH₂CH₃ |
| A-206 | C(CH₂CH₃)═CHCH₂CH₃ |
| A-207 | CH(CH₂CH₃)CH═CHCH₃ |
| A-208 | CH(CH₂CH₃)CH₂CH═CH₂ |
| A-209 | CH₂C(CH₃)₂CH═CH₂ |
| A-210 | C(═CH₂)CH(CH₃)CH₂CH₃ |
| A-211 | C(CH₃)═C(CH₃)CH₂CH₃ |
| A-212 | CH(CH₃)C(═CH₂)CH₂CH₃ |
| A-213 | CH(CH₃)C(CH₃)═CHCH₃ |
| A-214 | CH(CH₃)CH(CH₃)CH═CH₂ |
| A-215 | C(CH₃)₂CH═CHCH₃ |
| A-216 | C(CH₃)₂CH₂CH═CH₂ |
| A-217 | C(═CH₂)C(CH₃)₃ |
| A-218 | C(═CHCH₃)CH(CH₃)CH₃ |
| A-219 | CH(CH═CH₂)CH(CH₃)CH₃ |
| A-220 | C(CH₂CH₃)═C(CH₃)CH₃ |
| A-221 | CH(CH₂CH₃)C(═CH₂)CH₃ |
| A-222 | C(CH₃)₂C(═CH₂)CH₃ |
| A-223 | C(CH₃)(CH═CH₂)CH₂CH₃ |
| A-224 | C(CH₃)(CH₂CH₃)CH₂CH₂CH₃ |
| A-225 | CH(CH₂CH₃)CH(CH₃)CH₂CH₃ |
| A-226 | CH(CH₂CH₃)CH₂CH(CH₃)CH₃ |
| A-227 | C(CH₃)₂C(CH₃)₃ |
| A-228 | C(CH₂CH₃)₂C(CH₃)₃ |
| A-229 | C(CH₂CH₃)(CH₃)CH(CH₃)₂ |
| A-230 | CH(CH(CH₃)₂)CH(CH₃)₂ |
| A-231 | CH═CHCH₂CH₂CH₂CH₂CH₃ |
| A-232 | CH₂CH═CHCH₂CH₂CH₂CH₃ |
| A-233 | CH₂CH₂CH═CHCH₂CH₂CH₃ |
| A-234 | CH₂CH₂CH₂CH═CHCH₂CH₃ |
| A-235 | CH₂CH₂CH₂CH₂CH═CH—CH₃ |
| A-236 | CH₂CH₂CH₂CH₂CH₂CH═CH₂ |
| A-237 | CH═CHCH₂CH₂CH(CH₃)CH₃ |
| A-238 | CH₂CH═CHCH₂CH(CH₃)CH₃ |
| A-239 | CH₂CH₂CH═CHCH(CH₃)CH₃ |
| A-240 | CH₂CH₂CH₂CH═C(CH₃)CH₃ |
| A-241 | CH₂CH₂CH₂CH₂C(═CH₂)CH₃ |
| A-242 | CH═CHCH₂CH(CH₃)CH₂CH₃ |
| A-243 | CH₂CH═CHCH(CH₃)CH₂CH₃ |
| A-244 | CH₂CH₂CH═C(CH₃)CH₂CH₃ |
| A-245 | CH₂CH₂CH₂C(═CH₂)CH₂CH₃ |
| A-246 | CH₂CH₂CH₂C(CH₃)═CHCH₃ |
| A-247 | CH₂CH₂CH₂CH(CH₃)CH═CH₂ |
| A-248 | CH═CHCH(CH₃)CH₂CH₂CH₃ |
| A-249 | CH₂CH═C(CH₃)CH₂CH₂CH₃ |
| A-250 | CH₂CH₂C(═CH₂)CH₂CH₂CH₃ |
| A-251 | CH₂CH₂C(CH₃)═CHCH₂CH₃ |
| A-252 | CH₂CH₂CH(CH₃)CH═CHCH₃ |
| A-253 | CH₂CH₂CH(CH₃)CH₂CH═CH₂ |
| A-254 | CH═C(CH₃)CH₂CH₂CH₂CH₃ |
| A-255 | CH₂C(═CH₂)CH₂CH₂CH₂CH₃ |
| A-256 | CH₂C(CH₃)═CHCH₂CH₂CH₃ |
| A-257 | CH₂CH(CH₃)CH═CHCH₂CH₃ |
| A-258 | CH₂CH(CH₃)CH₂CH═CHCH₃ |
| A-259 | CH₂CH(CH₃)CH₂CH₂CH═CH₂ |
| A-260 | C(═CH₂)CH₂CH₂CH₂CH₂CH₃ |
| A-261 | C(CH₃)═CHCH₂CH₂CH₂CH₃ |
| A-262 | CH(CH₃)CH═CHCH₂CH₂CH₃ |
| A-263 | CH(CH₃)CH₂CH═CHCH₂CH₃ |
| A-264 | CH(CH₃)CH₂CH₂CH═CHCH₃ |
| A-265 | CH(CH₃)CH₂CH₂CH₂CH═CH₂ |
| A-266 | CH═CHCH₂C(CH₃)₃ |
| A-267 | CH₂CH═CHC(CH₃)₃ |
| A-268 | CH═CHCH(CH₃)CH(CH₃)₂ |
| A-269 | CH₂CH═C(CH₃)CH(CH₃)₂ |
| A-270 | CH₂CH₂C(═CH₂)CH(CH₃)₂ |
| A-271 | CH₂CH₂C(CH₃)═C(CH₃)₂ |
| A-272 | CH₂CH₂CH(CH₃)C(═CH₂)(CH₃) |
| A-273 | CH═C(CH₃)CH₂CH(CH₃)₂ |
| A-274 | CH₂C(═CH₂)CH₂CH(CH₃)₂ |
| A-275 | CH₂C(CH₃)═CHCH(CH₃)₂ |
| A-276 | CH₂CH(CH₃)CH═C(CH₃)₂ |
| A-277 | CH₂CH(CH₃)CH₂C(═CH₂)CH₃ |
| A-278 | C(═CH₂)CH₂CH₂CH(CH₃)₂ |
| A-279 | C(CH₃)═CHCH₂CH(CH₃)₂ |
| A-280 | CH(CH₃)CH═CHCH(CH₃)₂ |
| A-281 | CH(CH₃)CH₂CH═C(CH₃)₂ |
| A-282 | CH(CH₃)CH₂CH₂C(═CH₂)CH₃ |
| A-283 | CH═CHC(CH₃)₂CH₂CH₃ |
| A-284 | CH₂CH₂C(CH₃)₂CH═CH₂ |
| A-285 | CH═C(CH₃)CH(CH₃)CH₂CH₃ |
| A-286 | CH₂C(═CH₂)CH(CH₃)CH₂CH₃ |
| A-287 | CH₂C(CH₃)═C(CH₃)CH₂CH₃ |
| A-288 | CH₂CH(CH₃)C(═CH₂)CH₂CH₃ |
| A-289 | CH₂CH(CH₃)C(CH₃)═CHCH₃ |
| A-290 | CH₂CH(CH₃)CH(CH₃)CH═CH₂ |
| A-291 | C(═CH₂)CH₂CH(CH₃)CH₂CH₃ |
| A-292 | C(CH₃)═CHCH(CH₃)CH₂CH₃ |
| A-293 | CH(CH₃)CH═C(CH₃)CH₂CH₃ |
| A-294 | CH(CH₃)CH₂C(═CH₂)CH₂CH₃ |
| A-295 | CH(CH₃)CH₂C(CH₃)═CHCH₃ |
| A-296 | CH(CH₃)CH₂CH(CH₃)CH═CH₂ |
| A-297 | CH₂C(CH₃)₂CH═CHCH₃ |
| A-298 | CH₂C(CH₃)₂CH₂CH═CH₂ |
| A-299 | C(═CH₂)CH(CH₃)CH₂CH₂CH₃ |
| A-300 | C(CH₃)═C(CH₃)CH₂CH₂CH₃ |
| A-301 | CH(CH₃)C(═CH₂)CH₂CH₂CH₃ |
| A-302 | CH(CH₃)C(CH₃)═CHCH₂CH₃ |
| A-303 | CH(CH₃)CH(CH₃)CH═CHCH₃ |
| A-304 | CH(CH₃)CH(CH₃)CH₂CH═CH₂ |
| A-305 | C(CH₃)₂CH═CHCH₂CH₃ |
| A-306 | C(CH₃)₂CH₂CH═CHCH₃ |
| A-307 | C(CH₃)₂CH₂CH₂CH═CH₂ |
| A-308 | CH═CHCH(CH₂CH₃)CH₂CH₃ |
| A-309 | CH₂CH═C(CH₂CH₃)CH₂CH₃ |
| A-310 | CH₂CH₂C(═CHCH₃)CH₂CH₃ |
| A-311 | CH₂CH₂CH(CH═CH₂)CH₂CH₃ |
| A-312 | CH═C(CH₂CH₃)CH₂CH₂CH₃ |
| A-313 | CH₂C(═CHCH₃)CH₂CH₂CH₃ |
| A-314 | CH₂CH(CH═CH₂)CH₂CH₂CH₃ |
| A-315 | CH₂C(CH₂CH₃)═CHCH₂CH₃ |
| A-316 | CH₂CH(CH₂CH₃)CH═CHCH₃ |
| A-317 | CH₂CH(CH₂CH₃)CHCH═CH₂ |
| A-318 | C(═CHCH₃)CH₂CH₂CH₂CH₃ |
| A-319 | CH(CH═CH₂)CH₂CH₂CH₂CH₃ |
| A-320 | C(CH₂CH₃)═CHCH₂CH₂CH₃ |
| A-321 | CH(CH₂CH₃)CH═CHCH₂CH₃ |
| A-322 | CH(CH₂CH₃)CH₂CH═CHCH₃ |
| A-323 | CH(CH₂CH₃)CH₂CH₂CH═CH₂ |
| A-324 | C(═CHCH₂CH₃)CH₂CH₂CH₃ |
| A-325 | CH(CH═CHCH₃)CH₂CH₂CH₃ |
| A-326 | CH(CH₂CH═CH₂)CH₂CH₂CH₃ |
| A-327 | CH═C(CH₃)C(CH₃)₃ |
| A-328 | CH₂C(═CH₂)C(CH₃)₃ |
| A-329 | CH₂C(CH₃)₂CH(═CH₂)CH₃ |
| A-330 | C(═CH₂)CH(CH₃)CH(CH₃)CH₃ |
| A-331 | C(CH₃)═C(CH₃)CH(CH₃)CH₃ |
| A-332 | CH(CH₃)C(═CH₂)CH(CH₃)CH₃ |
| A-333 | CH(CH₃)C(CH₃)═C(CH₃)CH₃ |
| A-334 | CH(CH₃)CH(CH₃)C(═CH₂)CH₃ |
| A-335 | C(CH₃)₂CH═C(CH₃)CH₃ |
| A-336 | C(CH₃)₂CH₂C(═CH₂)CH₃ |
| A-337 | C(CH₃)₂C(═CH₂)CH₂CH₃ |
| A-338 | C(CH₃)₂C(CH₃)═CHCH₃ |
| A-339 | C(CH₃)₂CH(CH₃)CH═CH₂ |
| A-340 | CH(CH₂CH₃)CH₂CH(CH₃)CH₃ |
| A-341 | CH(CH₂CH₃)CH(CH₃)CH₂CH₃ |
| A-342 | C(CH₃)(CH₂CH₃)CH₂CH₂CH₃ |
| A-343 | CH(i-C₃H₇)CH₂CH₂CH₃ |
| A-344 | CH═C(CH₂CH₃)CH(CH₃)CH₃ |
| A-345 | CH₂C(═CHCH₃)CH(CH₃)CH₃ |
| A-346 | CH₂CH(CH═CH₂)CH(CH₃)CH₃ |
| A-347 | CH₂C(CH₂CH₃)═C(CH₃)CH₃ |
| A-348 | CH₂CH(CH₂CH₃)C(═CH₂)CH₃ |
| A-349 | CH₂C(CH₃)(CH═CH₂)CH₂CH₃ |
| A-350 | C(═CH₂)CH(CH₂CH₃)CH₂CH₃ |
| A-351 | C(CH₃)═C(CH₂CH₃)CH₂CH₃ |
| A-352 | CH(CH₃)C(═CHCH₃)CH₂CH₃ |
| A-353 | CH(CH₃)CH(CH═CH₂)CH₂CH₃ |
| A-354 | CH═C(CH₂CH₃)CH(CH₃)CH₃ |
| A-355 | CH₂C(═CHCH₃)CH(CH₃)CH₃ |
| A-356 | CH₂CH(CH═CH₂)CH(CH₃)CH₃ |
| A-357 | CH₂C(CH₂CH₃)═(CH₃)CH₃ |
| A-358 | CH₂CH(CH₂CH₃)C(═CH₂)CH₃ |
| A-359 | C(═CHCH₃)CH₂CH(CH₃)CH₃ |
| A-360 | CH(CH═CH₂)CH₂CH(CH₃)CH₃ |
| A-361 | C(CH₂CH₃)═CHCH(CH₃)CH₃ |
| A-362 | CH(CH₂CH₃)CH═C(CH₃)CH₃ |
| A-363 | CH(CH₂CH₃)CH₂C(═CH₂)CH₃ |
| A-364 | C(═CHCH₃)CH(CH₃)CH₂CH₃ |
| A-365 | CH(CH═CH₂)CH(CH₃)CH₂CH₃ |
| A-366 | C(CH₂CH₃)═C(CH₃)CH₂CH₃ |
| A-367 | CH(CH₂CH₃)C(═CH₂)CH₂CH₃ |
| A-368 | CH(CH₂CH₃)C(CH₃)═CHCH₃ |
| A-369 | CH(CH₂CH₃)CH(CH₃)CH═CH₂ |
| A-370 | C(CH₃)(CH═CH₂)CH₂CH₂CH₃ |
| A-371 | C(CH₃)(CH₂CH₃)CH═CHCH₃ |
| A-372 | C(CH₃)(CH₂CH₃)CH₂CH═CH₂ |
| A-373 | C[═C(CH₃)CH₃]CH₂CH₂CH₃ |
| A-374 | CH[C(═CH₂)CH₃]CH₂CH₂CH₃ |
| A-375 | C(i-C₃H₇)═CHCH₂CH₃ |
| A-376 | CH(i-C₃H₇)CH═CHCH₃ |
| A-377 | CH(i-C₃H₇)CH₂CH═CH₂ |
| A-378 | C(═CHCH₃)C(CH₃)₃ |
| A-379 | CH(CH═CH₂)C(CH₃)₃ |
| A-380 | C(CH₃)(CH═CH₂)CH(CH₃)CH₃ |
| A-381 | C(CH₃)(CH₂CH₃)C(═CH₂)CH₃ |
| A-382 | 2-CH₃-cyclohex-1-enyl |
| A-383 | [2-(═CH₂)]-cyclo-C₆H₉ |
| A-384 | 2-CH₃-cyclohex-2-enyl |
| A-385 | 2-CH₃-cyclohex-3-enyl |
| A-386 | 2-CH₃-cyclohex-4-enyl |
| A-387 | 2-CH₃-cyclohex-5-enyl |
| A-388 | 2-CH₃-cyclohex-6-enyl |
| A-389 | 3-CH₃-cyclohex-1-enyl |
| A-390 | 3-CH₃-cyclohex-2-enyl |
| A-391 | [3-(═CH₂)]-cyclo-C₆H₉ |
| A-392 | 3-CH₃-cyclohex-3-enyl |
| A-393 | 3-CH₃-cyclohex-4-enyl |
| A-394 | 3-CH₃-cyclohex-5-enyl |
| A-395 | 3-CH₃-cyclohex-6-enyl |
| A-396 | 4-CH₃-cyclohex-1-enyl |
| A-397 | 4-CH₃-cyclohex-2-enyl |
| A-398 | 4-CH₃-cyclohex-3-enyl |
| A-399 | [4-(═CH₂)]-cyclo-C₆H₉ |
| A-400 | C₆H₅ |
| A-401 | 4-CH₃—C₆H₄ |
| A-402 | 3-CH₃—C₆H₄ |
| A-403 | 2-CH₃—C₆H₄ |
| A-404 | 4-C₂H₅—C₆H₄ |
| A-405 | 4-(CH₂)₂CH₃—C₆H₄ |
| A-406 | 4-CH(CH₃)₂—C₆H₄ |
| A-407 | 4-(CH₂)₃CH₃—C₆H₄ |
| A-408 | 4-CH(CH₃)C₂H₅—C₆H₄ |
| A-409 | 4-CH₂CH(CH₃)₂—C₆H₄ |
| A-410 | 4-C(CH₃)₃—C₆H₄ |
| A-411 | 2,3-(CH₃)₂—C₆H₃ |
| A-412 | 2,4-(CH₃)₂—C₆H₃ |
| A-413 | 2,5-(CH₃)₂—C₆H₃ |
| A-414 | 3,4-(CH₃)₂—C₆H₃ |
| A-415 | 3,5-(CH₃)₂—C₆H₃ |
| A-416 | 4-C₆H₅—C₆H₄ |
| A-417 | 3-C₆H₅—C₆H₄ |
| A-418 | 4-Cl—C₆H₄ |
| A-419 | 3-Cl—C₆H₄ |
| A-420 | 2-Cl—C₆H₄ |
| A-421 | 4-F—C₆H₄ |
| A-422 | 3-F—C₆H₄ |
| A-423 | 2-F—C₆H₄ |
| A-424 | 2,3-Cl₂—C₆H₃ |
| A-425 | 2,4-Cl₂—C₆H₃ |
| A-426 | 2,5-Cl₂—C₆H₃ |
| A-427 | 3,4-Cl₂—C₆H₃ |
| A-428 | 3,5-Cl₂—C₆H₃ |
| A-429 | 2,3-F₂—C₆H₃ |
| A-430 | 2,4-F₂—C₆H₃ |
| A-431 | 2,5-F₂—C₆H₃ |
| A-432 | 3,4-F₂—C₆H₃ |
| A-433 | 3,5-F₂—C₆H₃ |
| A-434 | 2-Cl,4-F—C₆H₃ |
| A-435 | 4-CF₃—C₆H₄ |
| A-436 | 3-CF₃—C₆H₄ |
| A-437 | 2-CF₃—C₆H₄ |
| A-438 | 3,5-(CF₃)₂—C₆H₃ |
| A-439 | 4-OCH₃—C₆H₄ |
| A-440 | 3-OCH₃—C₆H₄ |
| A-441 | 2-OCH₃—C₆H₄ |
| A-442 | 2,3-(OCH₃)₂—C₆H₃ |
| A-443 | 2,4-(OCH₃)₂—C₆H₃ |
| A-444 | 2,5-(OCH₃)₂—C₆H₃ |
| A-445 | 3,4-(OCH₃)—C₆H₃ |
| A-446 | 3,5-(OCH₃)₂—C₆H₃ |
| A-447 | 2,3,4-(OCH₃)₃—C₆H₂ |
| A-448 | 3,4,5-(OCH₃)₃—C₆H₂ |
| A-449 | 4-OC₂H₅—C₆H₄ |
| A-450 | 3-OC₂H₅—C₆H₄ |
| A-451 | 2-OC₂H₅—C₆H₄ |
| A-452 | 4-SCH₃—C₆H₄ |
| A-453 | 3-SCH₃—C₆H₄ |
| A-454 | 2-SCH₃—C₆H₄ |
| A-455 | 4-SC₂H₅—C₆H₄ |
| A-456 | 4-OCF₃—C₆H₄ |
| A-457 | 3-OCF₃—C₆H₄ |
| A-458 | 2-OCF₃—C₆H₄ |
| A-459 | 4-CN—C₆H₄ |
| A-460 | 3-CN—C₆H₄ |
| A-461 | 2-CN—C₆H₄ |
| A-462 | 4-CHO—C₆H₄ |
| A-463 | 3-CHO—C₆H₄ |
| A-464 | 2-CHO—C₆H₄ |
| A-465 | 4-COCH₃—C₆H₄ |
| A-466 | 3-COCH₃—C₆H₄ |
| A-467 | 2-COCH₃—C₆H₄ |
| A-468 | 4-COOCH₃—C₆H₄ |
| A-469 | 4-COOC₂H₅—C₆H₄ |
| A-470 | 4-COOCH(CH₃)₂—C₆H₄ |
| A-471 | 4-CH₃,4-F—C₆H₃ |
| A-472 | 4-OH—C₆H₄ |
| A-473 | 3-OH—C₆H₄ |
| A-474 | 2-OH—C₆H₄ |
| A-475 | pyridin-2-yl |
| A-476 | pyridin-3-yl |
| A-477 | pyridin-4-yl |

In addition to the compounds individually listed in Tables 1 to 336, the invention also provides the corresponding derivatives in which R¹ and R⁴ are cyano.

In addition to the compounds individually listed in Tables 1 to 336, the invention also provides the corresponding derivatives in which R¹ and R⁴ are methoxy.

In addition to the compounds individually listed in Tables 1 to 336, the invention also provides the corresponding derivatives in which R¹ and R⁴ are methyl.

The compounds I are suitable as fungicides. They are distinguished by an excellent activity against a broad spectrum of phytopathogenic fungi from the class of the Ascomycetes, Deuteromycetes, Basidiomycetes and Peronosporomycetes (syn. Oomycetes). Some of them are systemically effective and can be used in crop protection as foliar fungicides, as fungicides for seed dressing and as soil fungicides.

They are particularly important in the control of a multitude of fungi on various cultivated plants, such as wheat, rye, barley, oats, rice, corn, grass, bananas, cotton, soybeans, coffee, sugar cane, vines, fruit and ornamental plants, and vegetables, such as cucumbers, beans, tomatoes, potatoes and cucurbits, and on the seeds of these plants. They may also be used in crops which are tolerant to attack by insects or fungi owing to breeding, including genetic engineering methods. Furthermore, they are suitable for controlling Botryosphaeria species, Cylindrocarpon species, Eutypa lata, Neonectria lifiodendri and Stereum hirsutum, which inter alia attack the wood or the roots of grapevines.

They are especially suitable for controlling the following plant diseases:

Alternaria species on vegetables, oilseed rape, sugar beet, fruit, rice, soybeans, and also on potatoes (for example A. solani or A. alternata) and tomatoes (for example A. solani or A. alternata) and Alternaria ssp. (black point) on wheat,

Aphanomyces species on sugar beet and vegetables,

Ascochyta species on cereals and vegetables, for example Ascochyta tritici (leaf spot) on wheat,

Bipolaris and Drechslera species on corn (for example D. maydis), cereals, rice and lawn,

Blumeria graminis (powdery mildew) on cereals (for example wheat or barley),

Botrytis cinerea (gray mold) on strawberries, vegetables, flowers, grapevines and wheat (ear mold),

Bremia lactucae on lettuce,

Cercospora species on corn, rice, sugar beet and, for example, Cercospora sojina (leaf blotch) or Cercospora kikuchii (leaf blotch) on soybeans,

Cladosporium herbarum (black mold) on wheat,

Cochliobolus species on corn, cereals (for example Cochliobolus sativus) and rice (for example Cochliobolus miyabeanus),

Colletotricum species on cotton and, for example, Colletotrichum truncatum (antracnose) on soybeans,

Corynespora cassficola (leaf blotch) on soybeans,

Dematophora necatrix (root/stem rot) on soybeans,

Diaporthe phaseolorum (stem disease) on soybeans,

Drechslera species, Pyrenophora species on corn, cereals, rice and lawn, on barley (for example D. teres) and on wheat (for example D. tritici-repentis),

Esca on grapevines, caused by Phaeoacremonium chlamydosporium, Ph. Aleophilum, and Formitipora punctata (syn. Phellinus punctatus),

Elsinoe ampelina on grapevines,

Epicoccum spp. (black head) on wheat,

Exserohlium species on corn,

Erysiphe cichoracearum and Sphaerotheca fuliginea on cucumbers,

Fusarium and Verticillium species on various plants: for example F. graminearum or F. culmorum (foot rot) on cereals (for example wheat or barley) or, for example, F. oxysporum on tomatoes and Fusarium solani (stem disease) on soybeans,

Gaeumanomyces graminis (take-all) on cereals (for example wheat or barley),

Gibberella species on cereals and rice (for example Gibberella fujikuroi),

Glomerella cingulata on grapevines and other plants,

Grainstaining complex on rice,

Guignardia budwelli on grapevines,

Helminthosporium species on corn and rice,

Isariopsis clavispora on grapevines,

Macrophomina phaseolina (root/stem rot) on soybeans,

Michrodochium nivale (pink snow mold) on cereals (for example wheat or barley),

Microsphaera diffusa (powdery mildew) on soybeans,

Mycosphaerella species on cereals, bananas and peanuts, such as, for example, M. graminicola on wheat or M. fijiensis on bananas,

Peronospora species on cabbage (for example P. brassicae), bulbous plants (for example P. destructor) and, for example, Peronospora manshurica (downy mildew) on soybeans,

Phakopsara pachyrhizi (soybean rust) and Phakopsara meibomiae (soybean rust) on soybeans,

Phialophora gregata (stem disease) on soybeans,

Phomopsis species on sunflowers, grapevines (for example P. viticola) and soybeans (for example Phomopsis phaseoli),

Phytophthora species on various plants, for example P. capsici on bell peppers,

Phytophthora megasperma (leaf/stem rot) on soybeans, Phytophthora infestans on potatoes and tomatoes,

Plasmopara viticola on grapevines,

Podosphaera leucotricha on apples,

Pseudocercosporella herpotrichoides (strawbreaker) on cereals (wheat or barley),

Pseudoperonospora on various plants, for example P. cubensis on cucumbers or P. humili on hops,

Pseudopezicula tracheiphllai on grapevines,

Puccima species on various plants, for example P. triticina, P. striformins, P. hordei or P. graminis on cereals (for example wheat or barley) or on asparagus (for example P. asparagi),

Pyriculana oryzae, Corticium sasakii, Sarocladium oryzae, S. attenuatum, Pyrenophora tritici-repentis (leaf spot) on wheat or Pyrenophora teres (net blotch) on barley,

Entyloma oryzae on rice,

Pyricularia grisea on lawn and cereals,

Pythium spp. on lawn, rice, corn, wheat, cotton, oilseed rape, sunflowers, sugar beet, vegetables and other plants (for example P. ultimum or P. aphanidermatum),

Ramularia collo-cygni (physiological leaf spots) on barley,

Rhizoctonia species on cotton, rice, potatoes, lawn, corn, oilseed rape, potatoes, sugar beet, vegetables and on various other plants, for example Rhizoctonia solani (root/stem rot) on soybeans or Rhizoctonia cerealis (yellow patch) on wheat or barley,

Rhynchosporium secalis on barley (scald), rye and triticale,

Sclerotinia species on oilseed rape, sunflowers and, for example, Sclerotinia scierotiorum (stem disease) or Sclerotinuia rolfsii (stem disease) on soybeans,

Septoria glycines (brown spot) on soybeans,

Septoria tritici (leaf spot) and Stagonospora nodorum on wheat,

Erysiphe (syn. Uncinula) necator on grapevines,

Setospaena species on corn and lawn,

Sphacelotheca reilinia on corn,

Stagonospora nodorum (glume blotch) on wheat,

Thievaliopsis species on soybeans and cotton,

Tilletia species on cereals,

Typhula incarnata (snow mold) on wheat or barley,

Ustilago species on cereals, corn (for example U. maydis) and sugar cane,

Venturia species (scab) on apples (for example V. inaequalis) and pears.

The compounds I are furthermore suitable for controlling harmful fungi in the protection of materials (for example wood, paper, paint dispersions, fibers or fabrics) and in the protection of stored products. In the protection of wood, particular attention is paid to the following harmful fungi: Ascomycetes, such as Ophiostoma spp., Ceratocystis spp., Aureobasidium pullulans, Sclerophoma spp., Chaetomium spp., Humicola spp., Petriella spp., Trichurus spp.; Basidiomycetes, such as Coniophora spp., Coriolus spp., Gloeophyllum spp., Lentinus spp., Pleurotus spp., Poria spp., Serpula spp. and Tyromyces spp., Deuteromycetes, such as Aspergillus spp., Cladosporium spp., Penicillium spp., Trichoderma spp., Alternaria spp., Paecilomyces spp. and Zygomycetes, such as Mucor spp., additionally in the protection of materials the following yeasts: Candida spp. and Saccharomyces cerevisae.

The compounds I are employed by treating the fungi or the plants, seeds or materials to be protected against fungal attack or the soil with a fungicidally effective amount of the active compounds. Application can be both before and after the infection of the materials, plants or seeds by the fungi.

The fungicidal compositions generally comprise between 0.1 and 95% by weight, preferably between 0.5 and 90% by weight, of active compound.

When employed in crop protection, the application rates are, depending on the kind of effect desired, between 0.01 and 2.0 kg of active compound per ha.

In seed treatment, the amounts of active compound required are generally from 1 to 1000 g/100 kg of seed, preferably from 5 to 100 g/100 kg of seed.

When used in the protection of materials or stored products, the active compound application rate depends on the kind of application area and on the desired effect. Amounts typically applied in the protection of materials are, for example, from 0.001 g to 2 kg, preferably from 0.005 g to 1 kg, of active compound per cubic meter of treated material.

The compounds of the formula I can be present in different crystal modifications which may differ in their biological activity. They are likewise subject matter of the present invention.

The compounds I can be converted into the customary formulations, for example solutions, emulsions, suspensions, dusts, powders, pastes or granules. The use form depends on the particular intended purpose; in each case, it should ensure a fine and even distribution of the compound according to the invention.

The formulations are prepared in a known manner, for example by extending the active compounds with solvents and carriers or solvents or carriers, if desired using further auxiliaries such as emulsifiers and dispersants. Here, individual compounds may also have various functions. Solvents, carriers and auxiliaries suitable for this purpose are essentially:
water, aromatic solvents (for example Solvesso® products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used.
carriers such as ground natural minerals (for example kaolins, clays, talc, chalk) and ground synthetic minerals (for example highly disperse silica, silicates);

emulsifiers such as nonionogenic and anionic emulsifiers (for example polyoxyethylene fatty alcohol ethers, alkylsulfonates and arylsulfonates) and dispersants such as lignosulfite waste liquors and methylcellulose.

The compositions according to the invention can be formulated in solid form or in liquid form. Depending on the embodiment, the compositions according to the invention may also comprise auxiliaries and/or carriers customary in crop protection compositions or in compositions for the protection of materials. The auxiliaries include in particular conventional surface-active substances and other additives and carriers customary in crop protection and in the protection of materials, which compounds may be solid or liquid. The surface-active substances include in particular surfactants, especially those having wetting agent properties. The other auxiliaries (additives) include in particular thickeners, antifoams, preservatives, antifreeze agents, stabilizers, anticaking agents or powder-flow aids and buffers.

Conventional surface-active substances which are usable in principle are anionic, nonionic and amphoteric surfactants including polymer surfactants, and the molecular weight of the surfactants will typically not exceed a value of 2000 Dalton and in particular 1000 Dalton (number-average).

The anionic surfactants include, for example, carboxylates, in particular alkali metal, alkaline earth metal, and ammonium salts of fatty acids, for example potassium stearate, which are usually also referred to as soaps; acyl glutamates; sarcosinates, for example sodium lauroyl sarcosinate; taurates; methylcelluloses; alkyl phosphates, in particular alkyl esters of mono- and diphosphoric acid; sulfates, in particular alkyl sulfates and alkyl ether sulfates; sulfonates, further alkyl sulfonates and alkylaryl sulfonates, in particular alkali metal, alkaline earth metal and ammonium salts of arylsulfonic acids and of alkyl-substituted arylsulfonic acids, alkylbenzenesulfonic acids, such as, for example, lignol and phenolsulfonic acid, naphthalene- and dibutylnaphthalenesulfonic acids, or dodecylbenzenesulfonates, alkylnaphthalene-sulfonates, alkyl methyl ester sulfonates, condensates of sulfonated naphthalene and derivatives thereof with formaldehyde, condensates of naphthalene sulfonic acids, phenol- and/or phenolsulfonic acids with formaldehyde or with formaldehyde and urea, mono- or dialkyl sulfosuccinates; and also protein hydrolysates and lignosulfite waste liquors. The abovementioned sulfonic acids are advantageously used in the form of their neutral or, if appropriate, basic salts.

The nonionic surfactants include, for example:
fatty alcohol alkoxylates and oxoalcohol alkoxylates, in particular ethoxylates and propoxylates having degrees of alkoxylation of usually from 2 to 100 and in particular from 3 to 50, for example alkoxylates of C₈-C₃₀-alkanols or alk(adi)enols, for example of isotridecyl alcohol, lauryl alcohol, oleyl alcohol or stearyl alcohol, and their C₁-C₄-alkyl ethers and C₁-C₄-alkyl esters, for example their acetates;
alkoxylated animal and/or vegetable fats and/or oils, for example corn oil ethoxylates, castor oil ethoxylates, tallow fat ethoxylates,
glycerol esters, such as, for example, glycerol monostearate,
alkylphenol alkoxylates, such as, for example, ethoxylated isooctylphenol, octylphenol or nonylphenol, tributylphenol polyoxyethylene ether,
fatty amine alkoxylates, fatty acid amide alkoxylates and fatty acid diethanolamide alkoxylates, in particular their ethoxylates,
sugar surfactants, sorbitol esters, such as, for example, sorbitan fatty acid esters (sorbitan monooleate, sorbitan tristearate), polyoxyethylene sorbitan fatty acid esters, alkyl polyglycosides, N-alkylgluconamides,
alkyl methyl sulfoxides,
alkyldimethylphosphine oxides, such as, for example, tetradecyldimethylphosphine oxide.

The amphoteric surfactants include, for example, sulfobetaines, carboxybetaines and alkyldimethylamine oxides, for example tetradecyldimethylamine oxide.

Other surfactants which may be mentioned here by way of example are perfluoro surfactants, silicone surfactants, phospholipids, such as, for example, lecithin or chemically modified lecithins, amino acid surfactants, for example N-lauroylglutamate.

Suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dustable products can be prepared by mixing or concomitantly grinding the active components A and B and, if present further actives with at least one solid carrier.

Granules, for example coated granules, impregnated granules and homogeneous granules, can be prepared by binding the active compounds to at least one solid carrier. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

The formulations of the compounds according to the invention generally comprise from 0.01 to 95% by weight, preferably from 0.1 to 90% by weight, of the compounds I and II. Here, the active compounds are preferably employed in a purity of from 90% to 100%, preferably from 95% to 100%.

For the treatment of seed, the formulations in question give, after two- to tenfold dilution, active compound concentrations of from 0.01 to 60% by weight, preferably from 0.1 to 40% by weight, in the ready-to-use preparations.

In general, the formulations comprise from 0.01 to 95% by weight, preferably from 0.1 to 90% by weight, of the active compound. The active compounds are employed in a purity of from 90% to 100%, preferably 95% to 100% (according to NMR spectrum).

The following are examples of formulations: 1. Products for dilution with water

A Water-Soluble Concentrates (SL, LS)

10 parts by weight of the active compounds are dissolved in 90 parts by weight of water or in a water-soluble solvent. As an alternative, wetting agents or other auxiliaries are added. The active compound dissolves upon dilution with water. In this way, a formulation having a content of 10% by weight of active compound is obtained.

B Dispersible Concentrates (DC)

20 parts by weight of the active compounds are dissolved in 70 parts by weight of cyclohexanone with addition of 10 parts by weight of a dispersant, for example polyvinylpyrrolidone. Dilution with water gives a dispersion. The active compound content is 20% by weight

### C Emulsifiable Concentrates (EC)

15 parts by weight of the active compounds are dissolved in 75 parts by weight of xylene with addition of calcium dodecylbenzenesulfonate and castor oil ethoxylate (in each case 5 parts by weight). Dilution with water gives an emulsion. The formulation has an active compound content of 15% by weight.

D Emulsions (EW, EO, ES)

25 parts by weight of the active compounds are dissolved in 35 parts by weight of xylene with addition of calcium dodecylbenzenesulfonate and castor oil ethoxylate (in each case 5 parts by weight). This mixture is introduced into 30 parts by weight of water by means of an emulsifying machine (e.g. Ultraturrax) and made into a homogeneous emulsion. Dilution with water gives an emulsion. The formulation has an active compound content of 25% by weight.

E Suspensions (SC, OD, FS)

In an agitated ball mill, 20 parts by weight of the active compounds are comminuted with addition of 10 parts by weight of dispersants and wetting agents and 70 parts by weight of water or an organic solvent to give a fine active compound suspension. Dilution with water gives a stable suspension of the active compound. The active compound content in the formulation is 20% by weight.

F Water-Dispersible Granules and Water-Soluble Granules (WG, SG)

50 parts by weight of the active compounds are ground finely with addition of 50 parts by weight of dispersants and wetting agents and prepared as water-dispersible or water-soluble granules by means of technical appliances (for example extrusion, spray tower, fluidized bed). Dilution with water gives a stable dispersion or solution of the active compound. The formulation has an active compound content of 50% by weight.

G Water-Dispersible Powders and Water-Soluble Powders (WP, SP, SS, WS)

75 parts by weight of the active compounds are ground in a rotor-stator mill with addition of 25 parts by weight of dispersants, wetting agents and silica gel. Dilution with water gives a stable dispersion or solution of the active compound. The active compound content of the formulation is 75% by weight.

H Gel Formulations (GF)

In a ball mill, 20 parts by weight of the active compounds, 10 parts by weight of dispersant, 1 part by weight of gelling agent and 70 parts by weight of water or an organic solvent are ground to give a fine suspension. On dilution with water, a stable suspension having an active compound content of 20% by weight is obtained.

2. Products to be Applied Undiluted

I Dustable Powders (DP, DS)

5 parts by weight of the active compounds are ground finely and mixed intimately with 95 parts by weight of finely divided kaolin. This gives a dustable product having an active compound content of 5% by weight.

J Granules (GR, FG, GG, MG)

0.5 part by weight of the active compounds is ground finely and associated with 99.5 parts by weight of carriers. Current methods are extrusion, spray-drying and the fluidized bed. This gives granules to be applied undiluted having an active compound content of 0.5% by weight.

K ULV Solutions (UL)

10 parts by weight of the active compounds are dissolved in 90 parts by weight of an organic solvent, for example xylene. This gives a product to be applied undiluted having an active compound content of 10% by weight.

For seed treatment, use is usually made of water-soluble concentrates (LS), suspensions (FS), dustable powders (DS), water-dispersible and water-soluble powders (WS, SS), emulsions (ES), emulsifiable concentrates (EC) and gel formulations (GF). These formulations can be applied to the seed in undiluted form or, preferably, diluted. Application can be carried out prior to sowing.

The active compounds can be used as such, in the form of their formulations or of the use forms prepared therefrom, e.g. in the form of directly sprayable solutions, powders, suspensions or dispersions, emulsions, oil dispersions, pastes, dustable products, materials for broadcasting or granules, by means of spraying, atomizing, dusting, broadcasting or watering. The use forms depend entirely on the intended purposes; they should always ensure the finest possible distribution of the active compounds according to the invention.

Aqueous use forms can be prepared from emulsifiable concentrates, pastes or wettable powders (spray powders, oil dispersions) by addition of water. To prepare emulsions, pastes or oil dispersions, the substances can be homogenized in water, as such or dissolved in an oil or solvent, by means of wetting agents, tackifiers, dispersants or emulsifiers. However, it is also possible to prepare concentrates comprising active substance, wetting agent, tackifier, dispersant or emulsifier and possibly solvent or oil which are suitable for dilution with water.

The concentrations of active compound in the ready-for-use preparations can be varied within relatively wide ranges. In general, they are between 0.0001 and 10%, preferably between 0.01 and 1%.

The active compounds can also be used with great success in the ultra-low volume (ULV) process, it being possible to apply formulations with more than 95% by weight of active compound or even the active compound without additives.

Oils of various types, wetting agents, adjuvants, herbicides, fungicides, other pesticides and bactericides can be added to the active compounds, if appropriate also not until immediately before use (tank mix). These agents can be added to the compositions according to the invention in a weight ratio of 1:100 to 100:1, preferably of 1:10 to 10:1.

The following are particularly suitable as adjuvants in this context: organically modified polysiloxanes, for example Break Thru S 240®; alcohol alkoxylates, for example Atplus 245®, Atplus MBA 1303®, Plurafac LF 300® and Lutensol ON 30®; EO-PO block polymers, for example Pluronic RPE 2035® and Genapol B®; alcohol ethoxylates, for example Lutensol XP 80®; and sodium dioctylsulfosuccinate, for example Leophen RA®.

The compositions according to the invention in the application form as fungicides can also be present together with other active compounds, for example with herbicides, insecticides, growth regulators, fungicides or else with fertilizers. When mixing the compounds (I) or the compositions comprising them with one or more further active compounds, in particular fungicides, it is in many cases possible, for example, to widen the activity spectrum or to prevent the development of resistance. In many cases, synergistic effects are obtained. The fungicides are preferably selected from the following groups. Preferred representatives of these groups are listed in Table B.

Strobilurins, carboxamides, such as carboxanilides, carboxylic acid morpholides, benzamides, other carboxamides, azoles, such as triazoles, imidazoles, benzimidazoles, others, nitrogenous heterocyclyl compounds, such as pyridines, pyrimidines, pyrroles, morpholines, dicarboximides, other nitrogenous heterocyclyl compounds, thio- and dithiocarbamates, carbamates, guanidines, antibiotics, nitrophenyl derivatives, organometal compounds, sulfur-containing heterocyclyl compounds, organophosphorus compounds, organochlorine compounds, inorganic active compounds, other fungicides.

The present invention further relates, accordingly, to the compositions that are listed in Table B, each line of Table B corresponding to a fungicidal composition comprising a compound of the formula I (component 1), which is preferably one of the compounds described herein as being preferred, and comprising the further active compound indicated in each case in the row in question (component 2). According to one embodiment of the invention, component 1 in each row of Table B is in each case one of the compounds of the formula I that are specifically individualized in Tables 1 to 336.

TABLE B

| Row | Component 1 | Component 2 |
|---|---|---|
| B-1 | a compound of the formula I | azoxystrobin |
| B-2 | a compound of the formula I | dimoxystrobin |
| B-3 | a compound of the formula I | enestroburin |
| B-4 | a compound of the formula I | fluoxastrobin |
| B-5 | a compound of the formula I | kresoxim-methyl |
| B-6 | a compound of the formula I | metominostrobin |
| B-7 | a compound of the formula I | orysastrobin |
| B-8 | a compound of the formula I | picoxystrobin |
| B-9 | a compound of the formula I | pyraclostrobin |
| B-10 | a compound of the formula I | pyribencarb |
| B-11 | a compound of the formula I | trifloxystrobin |
| B-12 | a compound of the formula I | 2-(2-(6-(3-chloro-2-methylphenoxy)-5-fluoropyrimidin- |
| | | 4-yloxy)phenyl)-2-methoxyimino-N-methylacetamide |
| B-13 | a compound of the formula I | methyl 2-(ortho-((2,5-dimethylphenyloxy- |
| | | methylene)phenyl)-3-methoxyacrylate |
| B-14 | a compound of the formula I | methyl 3-methoxy-2-(2-(N-(4-methoxyphenyl)- |
| | | cyclopropanecarboximidoylsulfanyl- |
| | | methyl)phenyl)acrylate |
| B-15 | a compound of the formula I | benalaxyl |
| B-16 | a compound of the formula I | benalaxyl-M |
| B-17 | a compound of the formula I | benodanil |
| B-18 | a compound of the formula I | bixafen |
| B-19 | a compound of the formula I | boscalid |
| B-20 | a compound of the formula I | carboxin |
| B-21 | a compound of the formula I | fenfuram |
| B-22 | a compound of the formula I | fenhexamid |
| B-23 | a compound of the formula I | flutolanil |
| B-24 | a compound of the formula I | furametpyr |
| B-25 | a compound of the formula I | isotianil |
| B-26 | a compound of the formula I | kiralaxyl |
| B-27 | a compound of the formula I | mepronil |
| B-28 | a compound of the formula I | metalaxyl |
| B-29 | a compound of the formula I | ofurace |
| B-30 | a compound of the formula I | oxadixyl |
| B-31 | a compound of the formula I | oxycarboxin |
| B-32 | a compound of the formula I | penthiopyrad |
| B-33 | a compound of the formula I | thifluzamide |
| B-34 | a compound of the formula I | tecloftalam |
| B-35 | a compound of the formula I | tiadinil |
| B-36 | a compound of the formula I | 2-amino-4-methylthiazole-5-carboxanilide |
| B-37 | a compound of the formula I | 2-chloro-N-(1,1,3-trimethylindan-4-yl)-nicotinamide |
| B-38 | a compound of the formula I | N-(3′,4′-dichloro-5-fluorobiphenyl-2-yl)- |
| | | 3-difluoromethyl-1-methyl-1H-pyrazole-4-carboxamide |
| B-39 | a compound of the formula I | 5-fluoro-1,3-dimethyl-1H-pyrazole- |
| | | 4-carboxylic acid [2-(1,3-dimethylbutyl)- |
| | | phenyl]amide |
| B-40 | a compound of the formula I | N-(4′-chloro-3′,5-difluorobiphenyl-2-yl)- |
| | | 3-difluoromethyl-1-methyl-1H-pyrazole-4-carboxamide |
| B-41 | a compound of the formula I | N-(4′-chloro-3′,5-difluorobiphenyl-2-yl)- |
| | | 3-trifluoromethyl-1-methyl-1H-pyrazole-4-carboxamide |
| B-42 | a compound of the formula I | N-(3′,4′-dichloro-5-fluorobiphenyl-2-yl)- |
| | | 3-trifluoromethyl-1-methyl-1H-pyrazole-4-carboxamide |
| B-43 | a compound of the formula I | N-(3′,5-difluoro-4′-methylbiphenyl-2-yl)- |
| | | 3-difluoromethyl-1-methyl-1H-pyrazole-4-carboxamide |
| B-44 | a compound of the formula I | N-(3′,5-difluoro-4′-methylbiphenyl-2-yl)- |
| | | 3-trifluoromethyl-1-methyl-1H-pyrazole-4-carboxamide |
| B-45 | a compound of the formula I | N-(2-bicyclopropyl-2-ylphenyl)-3-difluoro- |
| | | methyl-1-methyl-1H-pyrazole-4-carboxamide |
| B-46 | a compound of the formula I | N-(cis-2-bicyclopropyl-2-ylphenyl)- |
| | | 3-difluoromethyl-1-methyl-1H-pyrazole-4-carboxamide |
| B-47 | a compound of the formula I | N-(trans-2-bicyclopropyl-2-ylphenyl)-3- |
| | | difluoromethyl-1-methyl-1H-pyrazole-4-carboxamide |
| B-48 | a compound of the formula I | dimethomorph |
| B-49 | a compound of the formula I | flumorph |
| B-50 | a compound of the formula I | flumetover |
| B-51 | a compound of the formula I | fluopicolide (picobenzamid) |
| B-52 | a compound of the formula I | fluopyram |
| B-53 | a compound of the formula I | zoxamide |
| B-54 | a compound of the formula I | N-(3-ethyl-3,5,5-trimethylcyclohexyl)- |
| | | 3-formylamino-2-hydroxybenzamide |
| B-55 | a compound of the formula I | carpropamid |
| B-56 | a compound of the formula I | diclocymet |
| B-57 | a compound of the formula I | mandipropamid |
| B-58 | a compound of the formula I | oxytetracyclin |
| B-59 | a compound of the formula I | silthiofam |
| B-60 | a compound of the formula I | N-(6-methoxypyridin-3-yl)cyclopropane-carboxamide |
| B-61 | a compound of the formula I | azaconazole |
| B-62 | a compound of the formula I | bitertanol |
| B-63 | a compound of the formula I | bromuconazole |
| B-64 | a compound of the formula I | cyproconazole |
| B-65 | a compound of the formula I | difenoconazole |
| B-66 | a compound of the formula I | diniconazole |
| B-67 | a compound of the formula I | diniconazole-M |
| B-68 | a compound of the formula I | enilconazole |
| B-69 | a compound of the formula I | epoxiconazole |
| B-70 | a compound of the formula I | fenbuconazole |
| B-71 | a compound of the formula I | flusilazole |
| B-72 | a compound of the formula I | fluquinconazole |
| B-73 | a compound of the formula I | flutriafol |
| B-74 | a compound of the formula I | hexaconazole |
| B-75 | a compound of the formula I | imibenconazole |
| B-76 | a compound of the formula I | ipconazole |
| B-77 | a compound of the formula I | metconazole |
| B-78 | a compound of the formula I | myclobutanil |
| B-79 | a compound of the formula I | oxpoconazole |
| B-80 | a compound of the formula I | paclobutrazole |
| B-81 | a compound of the formula I | penconazole |
| B-82 | a compound of the formula I | propiconazole |
| B-83 | a compound of the formula I | prothioconazole |
| B-84 | a compound of the formula I | simeconazole |
| B-85 | a compound of the formula I | tebuconazole |
| B-86 | a compound of the formula I | tetraconazole |
| B-87 | a compound of the formula I | triadimenol |
| B-88 | a compound of the formula I | triadimefon |
| B-89 | a compound of the formula I | triticonazole |
| B-90 | a compound of the formula I | uniconazole |
| B-91 | a compound of the formula I | 1-(4-chlorophenyl)-2-([1,2,4]triazol-1-yl)- |
| | | cycloheptanol |
| B-92 | a compound of the formula I | cyazofamid |
| B-93 | a compound of the formula I | imazalil |
| B-94 | a compound of the formula I | imazalil sulfate |
| B-95 | a compound of the formula I | pefurazoate |
| B-96 | a compound of the formula I | prochloraz |
| B-97 | a compound of the formula I | triflumizole |
| B-98 | a compound of the formula I | benomyl |
| B-99 | a compound of the formula I | carbendazim |
| B-100 | a compound of the formula I | fuberidazole |
| B-101 | a compound of the formula I | thiabendazole |
| B-102 | a compound of the formula I | ethaboxam |
| B-103 | a compound of the formula I | etridiazole |
| B-104 | a compound of the formula I | hymexazole |
| B-105 | a compound of the formula I | fluazinam |
| B-106 | a compound of the formula I | pyrifenox |
| B-107 | a compound of the formula I | 1-(4-chlorophenyl)-1-(propyn-2-yloxy)-3-(4-(3,4- |
| | | dimethoxyphenyl)isoxazol-5-yl)-propan-2-one |
| B-108 | a compound of the formula I | 3-[5-(4-chlorophenyl)-2,3-dimethyl- |
| | | isoxazolidin-3-yl]pyridine |
| B-109 | a compound of the formula I | 2,3,5,6-tetrachloro-4-methanesulfonyl-pyridine |
| B-110 | a compound of the formula I | 3,4,5-trichloropyridine-2,6-dicarbonitrile |
| B-111 | a compound of the formula I | N-(1-(5-bromo-3-chloropyridin-2-yl)ethyl)- |
| | | 2,4-dichloronicotinamide |
| B-112 | a compound of the formula I | N-((5-bromo-3-chloropyridin-2-yl)methyl)- |
| | | 2,4-dichloronicotinamide |
| B-113 | a compound of the formula I | bupirimate |
| B-114 | a compound of the formula I | cyprodinil |
| B-115 | a compound of the formula I | diflumetorim |
| B-116 | a compound of the formula I | ferimzone |
| B-117 | a compound of the formula I | fenarimol |
| B-118 | a compound of the formula I | mepanipyrim |
| B-119 | a compound of the formula I | nitrapyrin |
| B-120 | a compound of the formula I | nuarimol |
| B-121 | a compound of the formula I | pyrimethanil |
| B-122 | a compound of the formula I | fludioxonil |
| B-123 | a compound of the formula I | fenpiclonil |
| B-124 | a compound of the formula I | aldimorph |
| B-125 | a compound of the formula I | dodemorph |
| B-126 | a compound of the formula I | dodemorph acetate |
| B-127 | a compound of the formula I | fenpropimorph |
| B-128 | a compound of the formula I | tridemorph |
| B-129 | a compound of the formula I | fluoroimide |
| B-130 | a compound of the formula I | iprodione |
| B-131 | a compound of the formula I | procymidone |
| B-132 | a compound of the formula I | vinclozolin |
| B-133 | a compound of the formula I | acibenzolar-S-methyl |
| B-134 | a compound of the formula I | amisulbrom |
| B-135 | a compound of the formula I | anilazine |
| B-136 | a compound of the formula I | blasticidin-S |
| B-137 | a compound of the formula I | captan |
| B-138 | a compound of the formula I | captafol |
| B-139 | a compound of the formula I | chinomethionat |
| B-140 | a compound of the formula I | dazomet |
| B-141 | a compound of the formula I | debacarb |
| B-142 | a compound of the formula I | diclomezine |
| B-143 | a compound of the formula I | difenzoquat |
| B-144 | a compound of the formula I | difenzoquat methylsulfate |
| B-145 | a compound of the formula I | famoxadone |
| B-146 | a compound of the formula I | fenamidone |
| B-147 | a compound of the formula I | fenoxanil |
| B-148 | a compound of the formula I | fenpropidin |
| B-149 | a compound of the formula I | folpet |
| B-150 | a compound of the formula I | octhilinone |
| B-151 | a compound of the formula I | oxolinic acid |
| B-152 | a compound of the formula I | piperalin |
| B-153 | a compound of the formula I | probenazole |
| B-154 | a compound of the formula I | proquinazid |
| B-155 | a compound of the formula I | pyroquilon |
| B-156 | a compound of the formula I | quinoxyfen |
| B-157 | a compound of the formula I | triazoxide |
| B-158 | a compound of the formula I | tricyclazole |
| B-159 | a compound of the formula I | triforine |
| B-160 | a compound of the formula I | 5-chloro-7-(4-methylpiperidin-1-yl)- |
| | | 6-(2,4,6-trifluorophenyl)-[1,2,4]triazolo- |
| | | [1,5-a]pyrimidine |
| B-161 | a compound of the formula I | 2-butoxy-6-iodo-3-propylchromen-4-one |
| B-162 | a compound of the formula I | ferbam |
| B-163 | a compound of the formula I | mancozeb |
| B-164 | a compound of the formula I | maneb |
| B-165 | a compound of the formula I | metiram |
| B-166 | a compound of the formula I | metam |
| B-167 | a compound of the formula I | methasulfocarb |
| B-168 | a compound of the formula I | propineb |
| B-169 | a compound of the formula I | thiram |
| B-170 | a compound of the formula I | zineb |
| B-171 | a compound of the formula I | ziram |
| B-172 | a compound of the formula I | diethofencarb |
| B-173 | a compound of the formula I | flubenthiavalicarb |
| B-174 | a compound of the formula I | iprovalicarb |
| B-175 | a compound of the formula I | propamocarb |
| B-176 | a compound of the formula I | propamocarb hydrochloride |
| B-177 | a compound of the formula I | methyl 3-(4-chlorophenyl)-3-(2-isopropoxy- |
| | | carbonylamino-3-methylbutyrylamino)-propionate |
| B-178 | a compound of the formula I | valiphenal |
| B-179 | a compound of the formula I | 4-fluorophenyl N-(1-(1-(4-cyanophenyl)- |
| | | ethanesulfonyl)but-2-yl)carbamate |
| B-180 | a compound of the formula I | dodine |
| B-181 | a compound of the formula I | dodine free base |
| B-182 | a compound of the formula I | iminoctadine |
| B-183 | a compound of the formula I | iminoctadine triacetate |
| B-184 | a compound of the formula I | iminoctadine tris(albesilate) |
| B-185 | a compound of the formula I | guazatine |
| B-186 | a compound of the formula I | guazatine acetate |
| B-187 | a compound of the formula I | kasugamycin |
| B-188 | a compound of the formula I | kasugamycin hydrochloride hydrate |
| B-189 | a compound of the formula I | polyoxine |
| B-190 | a compound of the formula I | streptomycin |
| B-191 | a compound of the formula I | validamycin A |
| B-192 | a compound of the formula I | binapacryl |
| B-193 | a compound of the formula I | dicloran |
| B-194 | a compound of the formula I | dinobuton |
| B-195 | a compound of the formula I | dinocap |
| B-196 | a compound of the formula I | nitrothal-isopropyl |
| B-197 | a compound of the formula I | tecnazen |
| B-198 | a compound of the formula I | fentin acetate |
| B-199 | a compound of the formula I | fentin chloride |
| B-200 | a compound of the formula I | fentin hydroxide |
| B-201 | a compound of the formula I | isoprothiolane |
| B-202 | a compound of the formula I | dithianon |
| B-203 | a compound of the formula I | edifenphos |
| B-204 | a compound of the formula I | fosetyl |
| B-205 | a compound of the formula I | fosetyl aluminum |
| B-206 | a compound of the formula I | iprobenfos |
| B-207 | a compound of the formula I | pyrazophos |
| B-208 | a compound of the formula I | tolclofos-methyl |
| B-209 | a compound of the formula I | chlorothalonil |
| B-210 | a compound of the formula I | dichlofluanid |
| B-211 | a compound of the formula I | dichlorophen |
| B-212 | a compound of the formula I | flusulfamide |
| B-213 | a compound of the formula I | hexachlorobenzene |
| B-214 | a compound of the formula I | pencycuron |
| B-215 | a compound of the formula I | pentachlorophenol and salts thereof |
| B-216 | a compound of the formula I | phthalide |
| B-217 | a compound of the formula I | quintozene |
| B-218 | a compound of the formula I | thiophanate methyl |
| B-219 | a compound of the formula I | tolylfluanid |
| B-220 | a compound of the formula I | N-(4-chloro-2-nitrophenyl)-N-ethyl- |
| | | 4-methylbenzenesulfonamide |
| B-221 | a compound of the formula I | phosphorous acid and salts thereof |
| B-222 | a compound of the formula I | sulfur |
| B-223 | a compound of the formula I | Bordeaux mixture |
| B-224 | a compound of the formula I | copper acetate |
| B-225 | a compound of the formula I | copper hydroxide |
| B-226 | a compound of the formula I | copper oxychloride |
| B-227 | a compound of the formula I | basic copper sulfate |
| B-228 | a compound of the formula I | biphenyl |
| B-229 | a compound of the formula I | bronopol |
| B-230 | a compound of the formula I | cyflufenamid |
| B-231 | a compound of the formula I | cymoxanil |
| B-232 | a compound of the formula I | diphenylamine |
| B-233 | a compound of the formula I | metrafenone |
| B-234 | a compound of the formula I | mildiomycin |
| B-235 | a compound of the formula I | oxine-copper |
| B-236 | a compound of the formula I | prohexadione-calcium |
| B-237 | a compound of the formula I | spiroxamine |
| B-238 | a compound of the formula I | tolylfluanid |
| B-239 | a compound of the formula I | N-(cyclopropylmethoxyimino-(6-difluoro- |
| | | methoxy-2,3-difluorophenyl)methyl)-2-phenylacetamide |
| B-240 | a compound of the formula I | N′-(4-(4-chloro-3-trifluoromethylphenoxy)- |
| | | 2,5-dimethylphenyl)-N-ethyl-N-methyl-formamidine |
| B-241 | a compound of the formula I | N′-(4-(4-fluoro-3-trifluoromethylphenoxy)- |
| | | 2,5-dimethylphenyl)-N-ethyl-N-methyl-formamidine |
| B-242 | a compound of the formula I | N′-(2-methyl-5-trifluoromethyl-4-(3-tri- |
| | | methylsilanylpropoxy)phenyl)-N-ethyl-N-methylformamidine |
| B-243 | a compound of the formula I | N′-(5-difluoromethyl-2-methyl-4-(3-tri- |
| | | methylsilanylpropoxy)phenyl)-N-ethyl-N-methylformamidine |

The active compounds II, mentioned above as component 2, their preparation and their action against harmful fungi are generally known (cf.: http://www.hclrss.demon.co.uk/index.html); they are commercially available. The compounds named according to IUPAC, their preparation and their fungicidal action are likewise known [cf. EP-A 226 917; EP-A 10 28 125; EP-A 10 35 122; EP-A 12 01 648; WO 98/46608; WO 99/24413; WO 03/14103; WO 03/053145; WO 03/066609; WO 04/049804].

The present invention furthermore relates to a pharmaceutical composition comprising at least one pyridazine according to the invention and/or a pharmaceutically acceptable salt thereof and, if appropriate, at least one pharmaceutically acceptable carrier. The invention also relates to the pharmaceutical use of the pyridazines of the formula I according to the invention, in particular the pyridazines of the formula I described in the above description as being preferred, and/or their pharmaceutically acceptable salts, in particular their use for preparing a medicament for the treatment of cancer.

The pyridazines of the formula I according to the invention, in particular the pyridazines of the formula I according to the invention described in the above description as being preferred, and/or their pharmaceutically acceptable salts effectively inhibit the growth and/or the propagation of tumor cells, as can be demonstrated in standard tests with tumor cell lines, such as HeLa, MCF-7 and COLO 205. In particular, the pyrimidines of the formula I according to the invention generally have IC₅₀ values of <10⁻⁶ mol/l (i.e. <1 μM), preferably IC₅₀ values of <10⁻⁷ mol/l (i.e. <100 nM), for cell cycle inhibition in HeLa cells.

The pyridazines of the formula I according to the invention, in particular the pyridazines of the formula I according to the invention described in the above description as being preferred, and/or their pharmaceutically acceptable salts are suitable for the treatment, inhibiton or control of growth and/or propagation of tumor cells and the disorders associated therewith. Accordingly, they are suitable for cancer therapy in warm-blooded vertebrates, for example mammals and birds, in particular man, but also other mammals, in particular useful and domestic animals, such as dogs, cats, pigs, ruminants (cattle, sheep, goats, bison, etc.), horses and birds, such as chicken, turkey, ducks, geese, guineafowl and the like.

The pyridazines of the formula I according to the invention, especially the pyridazines of the formula I according to the invention described in the above description as being preferred, and/or their pharmaceutically acceptable salts are particularly suitable for the therapy of cancer or cancerous disorders of the following organs: breast, lung, intestine, prostate, skin (melanoma), kidney, bladder, mouth, larynx, esophagus, stomach, ovaries, pancreas, liver and brain.

In addition to the pyridazine I according to the invention and/or its pharmaceutically acceptable salt, the pharmaceutical compositions according to the invention comprise at least optionally a suitable carrier. Suitable carriers are, for example, the solvents, carriers, excipients, binders and the like customarily used for pharmaceutical formulations, which are described below in an exemplary manner for individual types of administration.

The compounds I according to the invention can be administered in a customary manner, for example orally, intravenously, intramuscularly or subcutaneously. For oral administration, the active compound can be mixed, for example, with an inert diluent or with an edible carrier; it can be embedded into a hard or soft gelatin capsule, it can be compressed to tablets or it can be mixed directly with the food/feed. The active compound can be mixed with excipients and administered in the form of indigestible tablets, buccal tablets, pastilles, pills, capsules, suspensions, potions, syrups and the like. Such preparations should contain at least 0.1% of active compound. The composition of the preparation may, of course, vary. It usually comprises from 2 to 60% by weight of active compound, based on the total weight of the preparation in question (dosage unit). Preferred preparations of the compound I according to the invention comprise from 10 to 1000 mg of active compound per oral dosage unit.

The tablets, pastilles, pills, capsules and the like may furthermore comprise the following components: binders, such as traganth, gum arabic, corn starch or gelatin, excipients, such as dicalcium phosphate, disintegrants, such as corn starch, potato starch, alginic acid and the like, glidants, such as magnesium stearate, sweeteners, such as sucrose, lactose or saccharin, and/or flavors, such as peppermint, vanilla and the like. The capsules may furthermore comprise a liquid carrier. Other substances which modify the properties of the dosage unit may also be used. For example, tablets, pills and capsules may be coated with shellac, sugar or mixtures thereof. In addition to the active compound, syrups or potions may also comprise sugar (or other sweeteners), methyl- or propylparaben as preservative, a colorant and/or a flavor. The components of the active compound preparations must, of course, be pharmaceutically pure and nontoxic at the quantities employed. Furthermore, the active compounds can be formulated as preparations with a controlled release of active compound, for example as delayed-release preparations.

The active compounds can also be administered parenterally or intraperitoneally. Solutions or suspensions of the active compounds or their salts can be prepared with water using suitable wetting agents, such as hydroxypropylcellulose. Dispersions can also be prepared using glycerol, liquid polyethylene glycols and mixtures thereof in oils. Frequently, these preparations furthermore comprise a preservative to prevent the growth of microorganisms.

Preparations intended for injections comprise sterile aqueous solutions and dispersions and also sterile powders for preparing sterile solutions and dispersions. The preparation has to be sufficiently liquid for injection. It has to be stable under the preparation and storage conditions and it has to be protected against contamination by microorganisms. The carrier may be a solvent or a dispersion medium, for example, water, ethanol, a polyol (for example glycerol, propylene glycol or liquid polyethylene glycol), a mixture thereof and/or a vegetable oil.

### SYNTHESIS EXAMPLES

With appropriate modification of the starting materials, the procedures given in the synthesis examples below were used to obtain further compounds I. The compounds produced in this manner are listed in the table below, together with physical data.

The HPLC retention times (RT) in the following examples were determined using the RP-18 column Chromolith Speed ROD (Merck KgaA, Germany), with the eluent acetonitrile+0.1% trifluoroacetic acid (TFA)/water+0.1% TFA in a gradient of from 5:95 to 95:5 in 5 minutes at 40° C. Mass spectrometry was effected using quadropole electrospray ionization, 80 V (positive mode)

### Example 1

### Preparation of (3-{-4-[3-chloro-5-(4-chlorophenyl)-6-methylpyridazin-4-yl]-3,5-difluorophenoxy}propyl)dimethylamine [I-1]

### Step 1: 4-[3-Chloro-5-(4-chlorophenyl)-6-methylpyridazin-4-yl)-3,5-difluorophenol

0.2 g of 3-chloro-5-(4-chlorophenyl)-4-(2,6-difluoro-4-methoxyphenyl)-6-methylpyridazine (cf. WO 2005/121104) and 1 ml of 1M BBr₃ solution in methylene chloride were stirred at 20 to 25° C. for about 6 hours. The reaction mixture was then hydrolyzed with water, and the aqueous phase was extracted with ethyl acetate. The combined organic phases were dried and freed from the solvent. This gave 0.2 g of the title compound as a lightly colored crystalline material.

¹H-NMR (CDCl₃, δ in ppm): 7.35 (d, 2H); 7.1 (s, br, 1H); 7.05 (d, 2H); 6.4 (d, 2H); 2.55 (s, 3H)

### Step 2: (3-{-4-[3-Chloro-5-(4-chlorophenyl)-6-methylpyridazin-4-yl]-3,5-difluorophenoxy}propyl)dimethylamine

0.2 g of diethyl azodicarboxylate was added to 0.2 g of 4-[3-chloro-5-(4-chlorophenyl)-6-methylpyridazin-4-yl)-3,5-difluorophenol (from step 1), 0.1 g of dimethylaminopropanol and 0.3 g of triphenylphosphine in 5 ml of tetrahydrofuran, and the mixture was stirred at from 20 to 25° C. for about 1 hour. The reaction mixture was concentrated and the residue was purified by MPLC on silica gel using cyclohexane/ethyl acetate mixtures. The residue obtained after concentration of the eluate crystallized and was digested with hexane/diisopropyl ether. This gave 0.13 g of the title compound as a colorless crystalline material of m.p. 112-113° C.

¹H-NMR (CDCl₃, δ in ppm): 7.3 (d, 2H); 7.0 (d, 2H); 6.4 (d, 2H); 3.95 (t, 2H); 2.55 (s, 3H); 2.4 (t, 2H); 2.25 (s, 6H); 1.9 (m, 2H)

TABLE I
Compounds of the formula I.A1

| | | | | | | Phys. data; (m.p. [° C.]; |
|---|---|---|---|---|---|---|
| No. | R¹ | R² | Lₘ | L¹ | R⁴ | HPLC/MS RT [min]/MS [m/z]) |
| I-1 | CH₃ | 4-Cl—C₆H₄ | 2,6-F₂ | —O—CH₂CH₂CH₂N(CH₃)₂ | Cl | |
| I-2 | CH₃ | C₆H₅ | 2,6-F₂ | —O—CH₂CH₂CH₂N(CH₃)₂ | Cl | |
| I-3 | CH₃ | C₆H₅ | 2,6-F₂ | —O—CH₂CH₂CH₂OH | Cl | |
| I-4 | CH₃ | 4-F—C₆H₄ | 2,6-F₂ | —O—CH₂CH₂CH₂N(CH₃)₂ | Cl | |
| I-5 | CH₃ | 4-F—C₆H₄ | 2,6-F₂ | —O—CH₂CH₂CH₂OH | Cl | |
| I-6 | CH₃ | 4-CH₃—C₆H₄ | 2,6-F₂ | —O—CH₂CH₂CH₂N(CH₃)₂ | Cl | |
| I-7 | CH₃ | 4-CH₃—C₆H₄ | 2,6-F₂ | —O—CH₂CH₂CH₂OH | Cl | |
| I-8 | CH₃ | 4-Cl—C₆H₄ | 2,6-F₂ | —O—CH₂CH₂CH₂NHCH₃ | Cl | 2.669/437.7 |
| I-9 | CH₃ | 4-Cl—C₆H₄ | 2,6-F₂ | —O—CH₂CH₂CH₂OH | Cl | 47-66 |
| I-10 | CH₃ | 4-Cl—C₆H₄ | 2,6-F₂ | —O—CH₂CH₂OH | Cl | 3.092/410.6 |
| I-11 | CH₃ | 4-Cl—C₆H₄ | 2,6-F₂ | —O—CH₂CH₂NHC(O)NH₂ | Cl | |
| I-12 | CH₃ | 4-Cl—C₆H₄ | 2,6-F₂ | —O—CH₂CH₂CH₂NHC(O)NH₂ | Cl | 158-164 |
| I-13 | CH₃ | 4-Cl—C₆H₄ | 2,6-F₂ | —O—CH₂CH₂CH₂OC(O)NH₂ | Cl | |
| I-14 | CH₃ | 4-Cl—C₆H₄ | 2,6-F₂ | —O—CH₂CH₂CH₂NHC(O)OCH₃ | Cl | 169-173 |
| I-15 | CH₃ | 4-Cl—C₆H₄ | 2,6-F₂ | —O—CH₂CH₂CH₂NHCHO | Cl | |
| I-16 | CH₃ | 4-Cl—C₆H₄ | 2,6-F₂ | —O—CH₂CH₂CH₂N(CH₃)CHO | Cl | 3.289/465.6 |
| I-17 | CH₃ | 4-Cl—C₆H₄ | 2,6-F₂ | —O—CH₂CH₂CH₂NHC(O)CH₃ | Cl | 65-70 |
| 1-18 | CH₃ | 4-Cl—C₆H₄ | 2,6-F₂ | 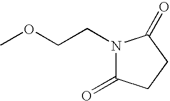 | Cl | 203-206 |

### Examples of the Action Against Harmful Fungi

The fungicidal action of the compounds of the formula I was demonstrated by the following experiments:

The active compounds were prepared as a stock solution comprising 25 mg of active compound which was made up to 10 ml using a mixture of acetone and/or DMSO and the emulsifier Uniperol® EL (wetting agent having emulsifying and dispersing action based on ethoxylated alkylphenols) in a volume ratio of solvent/emulsifier of 99/1. The mixture was then made up to 100 ml with water. This stock solution was diluted with the solvent/emulsifier/water mixture described to the concentration of active compounds stated below. Alternatively, the active compounds were used as a commercial finished formulation and diluted with water to the stated active compound concentration.

### Use Example 1

### Activity Against Late Blight on Tomatoes Caused by Phytophthora infestans, Protective Treatment

Leaves of potted tomato plants were sprayed to runoff point with an aqueous suspension having the active compound concentration stated below. The next day, the leaves were infected with an aqueous sporangia suspension of Phytophthora infestans. The plants were then placed in a water vapor-saturated chamber at temperatures between 18 and 20° C. After 6 days, the late blight on the untreated but infected control plants had developed to such an extent that the infection could be determined visually in %.

In this test, the plants treated with 250 ppm of the active compound I-1 showed no infection, whereas the untreated plants were 80% infected.

### Use Example 2

### Protective Activity Against Septoria Leaf Blotch of Wheat Caused by Septoria tritici

Leaves of potted wheat seedlings were inoculated with a spore suspension of the leaf blotch disease Septoria tritici. The test plants were then placed in a greenhouse at temperatures between 20 and 22° C. and a relative atmospheric humidity of close to 100% and then at temperatures between 16 and 18° C. and an atmospheric humidity of about 70% for 4 days. Six days after the inoculation, the plants were sprayed to runoff point with an aqueous active compound solution having the concentration stated below. After the spray coating had dried on, the plants were returned. After 14 days, the extent of the development of the disease was determined visually in % infection of the entire leaf area.

In this test, the plants which had been treated with 250 ppm of the active compound I-1 showed an infection of 10%, whereas the untreated plants were 90% infected. The active compounds were formulated separately as a stock solution having a concentration of 10 000 ppm in DMSO.

### Use Example 3

### Activity Against the Septoria Leaf Blotch Pathogen Septoria tritici the Microtiter Test

The stock solution is pipetted into a microtiter plate (MTP) and diluted with an aqueous malt-based fungus nutrient medium to the stated active compound concentration. An aqueous spore suspension of Septoria tritici was then added. The plates were placed in a water vapor-saturated chamber at temperatures of 18° C. Using an absorption photometer, the MTPs were measured at 405 nm on day 7 after the inoculation.

The measured parameters were compared to the growth of the active compound-free control variant (100%) and the fungus- and active compound-free blank value to determine the relative growth in % of the pathogens in the individual active compounds.

### Use Example 4

### Activity Against the Gray Mold Pathogen Botrytis cinerea in the Microtiter Test

The stock solution is pipetted into a microtiter plate (Mtp) and diluted with an aqueous malt-based fungus nutrient medium to the stated active compound concentration. An aqueous spore suspension of Botrytis cinerea was then added. The plates were placed in a water vapor-saturated chamber at temperatures of 18° C. Using an absorption photometer, the MTPs were measured at 405 nm on day 7 after the inoculation.

The measured parameters were compared to the growth of the active compound-free control variant and the fungus- and active compound-free blank value to determine the relative growth in % of the pathogens in the individual active compounds.

1-12. (canceled)

13. A compound of the formula I, 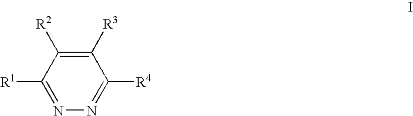
wherein the substituents have the following meaning:
R¹, R⁴ independently of one another are halogen, cyano, C₁-C₈-alkyl or C₁-C₈-alkoxy;
R² is C₁-C₁₂-alkyl, C₁-C₁₀-haloalkyl, C₂-C₁₀-alkenyl, C₂-C₁₀-haloalkenyl, C₂-C₁₀-alkynyl, C₂-C₁₀-haloalkynyl, C₃-C₆-cycloalkyl, C₃-C₈-cycloalkenyl, C₃-C₁₂-cycloalkenyl, C₃-C₆-halocycloalkyl, C₃-C₁₂-halocycloalkenyl, aryl or aromatic heterocycle which, in addition to carbon atoms, contains one to four nitrogen atoms or one to three nitrogen atoms and one sulfur or oxygen atom as ring members, wherein the aromatic groups are five-, six-, seven-, eight-, nine- or ten-membered ring systems; and R² may contain one, two, three or four identical or different groups R^{a} independently of one another selected from the group consisting of cyano, nitro, hydroxyl, carboxyl, C₁-C₆-alkyl, C₂-C₆-alkynyl, C₃-C₆-cycloalkyl, C₃-C₈-cycloalkenyl, C₁-C₆-alkoxy, C₂-C₆-alkenyloxy, C₃-C₆-alkynyloxy, C₃-C₆-cycloalkoxy, C₃-C₆-cycloalkenyloxy, C(O)R^{π}, C(O)OR^{π}, C(S)OR^{π}, C(O)SR^{π}, C(S)SR^{π}, OC(O)OR^{π}, C₁-C₆-alkylthio, amino, C₁-C₆-alkylamino, di-C₁-C₆-alkylamino, C₁-C₆-alkylene, oxy-C₁-C₄-alkylene, oxy-C₁-C₃-alkyleneoxy, wherein divalent groups may be attached to the same atom or to adjacent atoms, phenyl, naphthyl, a five-, six-, seven-, eight-, nine- or ten-membered saturated, partially unsaturated or aromatic heterocycle which, in addition to carbon atoms, contains one to four nitrogen atoms or one to three nitrogen atoms and one sulfur or oxygen atom as ring members;
R^{π} is C₁-C₈-alkyl, C₃-C₈-alkenyl, C₃-C₈-alkynyl, C₃-C₆-cycloalkyl or C₃-C₆-cycloalkenyl;
wherein the aliphatic, alicyclic or aromatic groups in groups R^{a} and R^{π} mentioned above for their part may be partially or fully halogenated and/or may carry one, two or three groups R^{b}:
R^{b} is halogen, cyano, nitro, hydroxyl, mercapto, amino, carboxyl, alkyl, haloalkyl, alkenyl, alkoxy, haloalkoxy, alkenyloxy, alkynyloxy, alkylthio, alkylamino, dialkylamino, formyl, alkylcarbonyl, alkylsulfonyl, alkylsulfoxyl, alkoxycarbonyl, alkylcarbonyloxy, alkoxycarbonyloxy, aminocarbonyl, aminothiocarbonyl, alkylaminocarbonyl, dialkylaminocarbonyl, alkylaminothiocarbonyl, dialkylaminothiocarbonyl, wherein the alkyl groups in these groups contain 1 to 6 carbon atoms and the alkenyl or alkynyl groups mentioned in these groups contain 2 to 8 carbon atoms; cycloalkyl, cycloalkoxy, heterocyclyl, heterocyclyloxy, wherein the cyclic systems contain 3 to 10 ring members; aryl, aryloxy, arylthio, aryl-C₁-C₆-alkoxy, hetaryl, hetaryloxy, hetarylthio, wherein the aryl radicals preferably contain 6 to 10 ring members and the hetaryl radicals 5 or 6 ring members, wherein the cyclic systems may be partially or fully halogenated and/or substituted by alkyl or haloalkyl groups;
R³ is phenyl or a 5- or 6-membered heteroaromatic group which, in addition to carbon atoms, contains 1, 2, 3 or 4 heteroatoms selected from the group consisting of O, S and N as ring members, wherein phenyl or the heteroaromatic group carries one substituents L¹ and optionally substituents Lₘ;
L¹ is a group of the formulae —Y¹—Y²-T, C(Rⁱ)═C(Rⁱⁱ)—Y¹—Y²-T or C≡C—Y¹—Y²-T,
wherein
Y¹ is CR^{h}Rⁱ, C(O)O, C(O)NR^{h}, O, NR^{h} or S(O)ᵣ;
Y² is C₁-C₈-alkylene, C₂-C₈-alkenylene or C₂-C₈-alkynylene, where Y² may be interrupted by one, two, three or four heteroatoms from the group consisting of NR^{h}, O and S(O)ᵣ and/or may contain one, two, three or four identical or different groups R^{a};
r is 0, 1 or 2;
T is OR^{h}, NR^{h}Rⁱ, C(O)OR^{h}, C(O)NR^{h}Rⁱ, C(NOR^{h})Rⁱ or T¹-C(=T²)-T³, wherein
T¹ is O or NR^{h};
T² is O, S or NR^{h};
T³ is R^{h}, OR^{h}, SR^{h} or NR^{h}Rⁱ;
independently, each R^{h} is hydrogen or has one of the meanings mentioned for R^{π}; and
Rⁱ, Rⁱⁱ independently of one another are one of the groups mentioned for R^{a};
L independently of one another are halogen, hydroxyl, cyanato (OCN), cyano, nitro, C₁-C₈-alkyl, C₁-C₈-haloalkyl, C₂-C₁₀-alkenyl, C₂-C₁₀-haloalkenyl, C₂-C₁₀-alkynyl, C₃-C₆-cycloalkyl, C₃-C₆-halocycloalkyl, C₃-C₆-cycloalkenyl, C₁-C₈-alkoxy, C₁-C₈-haloalkoxy, C₂-C₁₀-alkenyloxy, C₂-C₁₀-alkynyloxy, C₃-C₆-cycloalkyloxy, C₃-C₆-cycloalkenyloxy, amino, C₁-C₄-alkylamino, di-(C₁-C₄)-alkylamino, C(O)—R^{Φ}, C(S)—R^{Φ}, S(O)ₙ—R^{Φ}; C₁-C₈alkoxyimino-(C₁-C₈)-alkyl, C₂-C₁₀-alkenyloxyimino-(C₁-C₈)-alkyl, C₂-C₁₀-alkynyloxyimino-(C₁-C₈)-alkyl, C₂-C₁₀-alkynylcarbonyl, C₃-C₆-cycloalkylcarbonyl, or a five-, six-, seven-, eight-, nine- or ten-membered saturated, partially unsaturated or aromatic heterocycle which contains one, two, three or four heteroatoms from the group consisting of O, N and S;
R^{Φ} is hydrogen, C₁-C₄-alkyl, C₁-C₂-haloalkyl, C₁-C₄-alkoxy, C₂-C₄-alkenyloxy, C₂-C₄-alkynyloxy; where the groups R^{Φ} may be substituted by one, two or three identical or different groups R^{b}, as defined above;
n is zero, 1 or 2;
and
m is 1, 2, 3 or 4;
or an agriculturally acceptable salt thereof.

14. The compound of claim 13, wherein L^{I} is a group of the formula —Y¹—Y²-T.

15. The compound claim 13, wherein R¹ and R⁴ are halogen.

16. The compound of claim 13, wherein R² is alkyl or aryl.

17. The compound of claim 13, wherein R³ is phenyl.

18. The compound of claim 13, wherein R^{I} is alkyl, R² is optionally substituted phenyl, R⁴ is phenyl which is substituted by Lₘ and L¹ and R⁴ is halogen.

19. A process for preparing compounds of claim 13, wherein compounds of the formula II 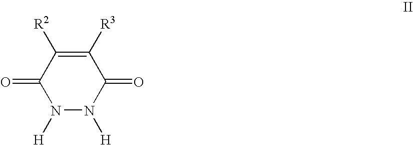
are reacted with halogenating agents, which compounds II are obtained by reacting compounds of the formula III 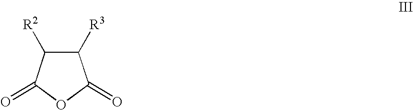
with hydrazine, which compounds of the formula III are obtained by oxidative cyclization of compounds of the formula IV 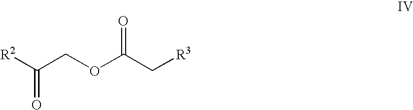
which are obtained by condensation of carboxylic acids of the formula V 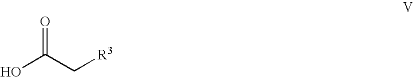
with halides of the formula VI, 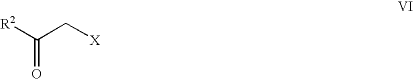
wherein X is halogen.

20. The compound of the formula II of claim 19.

21. A fungicidal composition comprising a solid or liquid carrier and a compound of claim 13.

22. The composition of claim 21, further comprising a second compound.

23. Seed comprising a compound of the formula I of claim 13 in an amount of from 1 to 1000 g per 100 kg.

24. A method for controlling phytopathogenic harmful fungi, wherein the fungi or the materials, plants, the soil or seed to be protected against fungal attack are/is treated with an effective amount of a compound of the formula I of claim 13.

25. A method for controlling phytopathogenic harmful fungi, wherein the fungi or the materials, plants, the soil or seed to be protected against fungal attack are/is treated with an effective amount of a compound of the formula I of claim 14.

26. A method for controlling phytopathogenic harmful fungi, wherein the fungi or the materials, plants, the soil or seed to be protected against fungal attack are/is treated with an effective amount of a compound of the formula I of claim 15.

27. A method for controlling phytopathogenic harmful fungi, wherein the fungi or the materials, plants, the soil or seed to be protected against fungal attack are/is treated with an effective amount of a compound of the formula I of claim 16.

28. A method for controlling phytopathogenic harmful fungi, wherein the fungi or the materials, plants, the soil or seed to be protected against fungal attack are/is treated with an effective amount of a compound of the formula I of claim 17.

29. A method for controlling phytopathogenic harmful fungi, wherein the fungi or the materials, plants, the soil or seed to be protected against fungal attack are/is treated with an effective amount of a compound of the formula I of claim 18.

30. A pharmaceutical composition comprising at least one compound of the formula I of claim 13 and/or at least one pharmaceutically acceptable salt thereof and optionally at least one pharmaceutically acceptable carrier.
